(19) United States
(12) Patent Application Publication
(10) Pub. No.: US 2023/0117860 A1
(43) Pub. Date: Apr. 20, 2023

(54) COMPOUND FOR ORGANIC OPTOELECTRONIC DEVICE, ORGANIC OPTOELECTRONIC DEVICE, AND DISPLAY DEVICE

(71) Applicant: SAMSUNG SDI CO., LTD., Yongin-si (KR)

(72) Inventors: Jonghoon KIM, Suwon-si (KR); Byoungkwan LEE, Suwon-si (KR); Mijin LEE, Suwon-si (KR); Youngmook LIM, Suwon-si (KR); Dong Min KANG, Suwon-si (KR); Hyung Sun KIM, Suwon-si (KR); Sung-Hyun JUNG, Suwon-si (KR); Ho Kuk JUNG, Suwon-si (KR); Youngkyoung JO, Suwon-si (KR); Bo won CHOI, Suwon-si (KR)

(21) Appl. No.: 17/901,908

(22) Filed: Sep. 2, 2022

(30) Foreign Application Priority Data
Sep. 3, 2021 (KR) 10-2021-0117781
Aug. 31, 2022 (KR) 10-2022-0110103

(51) Int. Cl. C07D 307/91; C07D 333/76; H01L 51/00; H01L 51/50

(52) U.S. Cl. CPC C07D 307/91; C07D 333/76; H01L 51/0073; H01L 51/0074; H01L 51/0054; H01L 51/5012; C07B 2200/05

(57) ABSTRACT

A compound for an organic optoelectronic device, an organic optoelectronic device including the same, and a display device, the compound being represented by Chemical Formula 1:

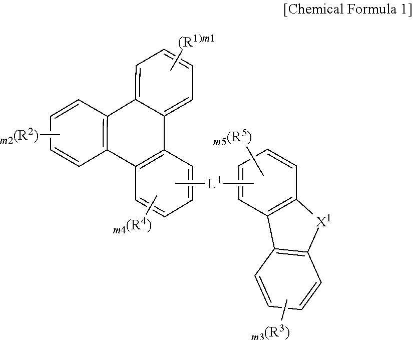

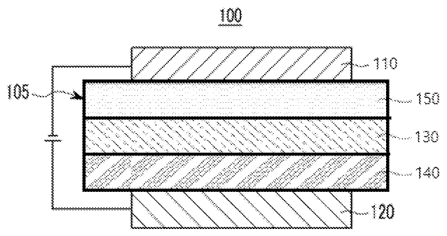

### CROSS-REFERENCE TO RELATED APPLICATION

This application claims priority to and the benefit of Korean Patent Application No. 10-2021-0117781 filed in the Korean Intellectual Property Office on Sep. 3, 2021, and Korean Patent Application No. 10-2022-0110103 filed in the Korean Intellectual Property Office on Aug. 31, 2022, the entire contents of which are incorporated herein by reference.

### BACKGROUND

### 1. Field

Embodiments relate to a compound for an organic optoelectronic device, an organic photoelectronic device, and a display device.

### 2. Description of the Related Art

An organic optoelectronic device (e.g., organic optoelectronic diode) is a device capable of converting electrical energy and optical energy to each other.

An organic optoelectronic device may be classified as follows in accordance with its driving principles. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device include an organic photoelectric element, an organic light emitting diode, an organic solar cell, and an organic photo conductor drum.

Of these, an organic light emitting diode (OLED) has recently drawn attention due to an increase in demand for flat panel displays. The organic light emitting diode is a device that converts electrical energy into light, and the performance of the organic light emitting diode is greatly influenced by an organic material between electrodes.

### SUMMARY

The embodiments may be realized by providing a compound for an organic optoelectronic device, the compound being represented by Chemical Formula 1:

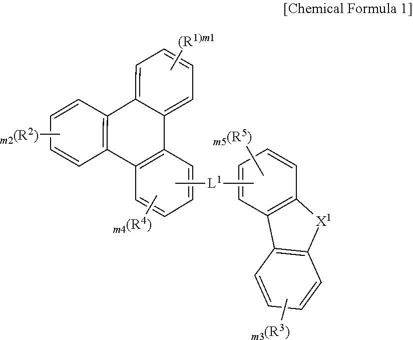

wherein, in Chemical Formula 1, X¹ is O or S, L¹ is a single bond, a substituted or unsubstituted C6 to C20 arylene group, a substituted or unsubstituted C2 to C20 heterocyclic group, or a combination thereof, R¹ to R⁵ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof, m1 to m3 are each independently an integer of 1 to 4, m4 and m5 are each independently an integer 1 to 3, and provided that at least one of the following conditions is met L¹ is a C6 to C20 arylene group substituted with at least one deuterium or a C2 to C20 heterocyclic group substituted with at least one deuterium; or at least one of R¹ to R⁵ is deuterium, a C6 to C30 aryl group substituted with at least one deuterium, or a C2 to C30 heterocyclic group substituted with at least one deuterium.

The embodiments may be realized by providing an organic optoelectronic device including an anode and a cathode facing each other, and at least one organic layer between the anode and the cathode, wherein the at least one organic layer includes the compound for an organic optoelectronic device according to an embodiment.

The embodiments may be realized by providing a display device including the organic optoelectronic device according to an embodiment.

### BRIEF DESCRIPTION OF THE DRAWING

Features will be apparent to those of skill in the art by describing in detail exemplary embodiments with reference to the attached drawing in which:

the FIGURE is a cross-sectional view illustrating an organic light emitting diode according to an embodiment.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figure, the dimensions of layers and regions may be exaggerated for clarity of illustration. It will also be understood that when a layer or element is referred to as being “on” another layer or element, it can be directly on the other layer or element, or intervening layers may also be present. In addition, it will also be understood that when a layer is referred to as being “between” two layers, it can be the only layer between the two layers, or one or more intervening layers may also be present. Like reference numerals refer to like elements throughout. As used herein, the term “or” is not an exclusive term, e.g., “A or B” would include A, B, or A and B.

In one example of the present disclosure, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In specific example of the present disclosure, the “substituted” refers to replacement of at least one hydrogen of a substituent or a compound by deuterium, a C1 to C30 alkyl group, a Cl to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In specific example of the present disclosure, the “substituted” refers to replacement of at least one hydrogen of a substituent or a compound by deuterium, a C1 to C20 alkyl group, a C6 to C30 aryl group, or a cyano group. In specific example of the present disclosure, the “substituted” refers to replacement of at least one hydrogen of a substituent or a compound by deuterium, a C1 to C5 alkyl group, a C6 to C18 aryl group, or a cyano group. In specific example of the present disclosure, the “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

“Unsubstituted” refers to non-replacement of a hydrogen atom by another substituent and remaining of the hydrogen atom.

As used herein, “hydrogen substitution (—H)” may include deuterium substitution (-D) or “tritium substitution (-T)”.

As used herein, when a definition is not otherwise provided, “hetero” refers to one including one to three heteroatoms selected from N, O, S, P, and Si, and remaining carbons in one functional group.

As used herein, “an aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quarterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “a heterocyclic group” is a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “a heteroaryl group” may refer to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzothiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, or a combination thereof, but is not limited thereto.

In the present specification, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to an embodiment is described.

A compound for an organic optoelectronic device according to an embodiment may be represented by, e.g., Chemical Formula 1.

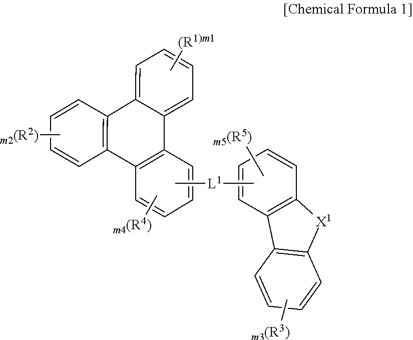

In Chemical Formula 1, X¹ may be, e.g., O or S.

L¹ may be or may include, e.g., a single bond, a substituted or unsubstituted C6 to C20 arylene group, a substituted or unsubstituted C2 to C20 heterocyclic group, or a combination thereof.

R¹ to R⁵ may each independently be or include, e.g., hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof.

m1 to m3 may each independently be, e.g., an integer of 1 to 4.

m4 and m5 may each independently be, e.g., an integer of 1 to 3.

In an implementation, the compound of Chemical Formula 1 may meet at least one of the following conditions:

L¹ may be, e.g., a C6 to C20 arylene group substituted with at least one deuterium or a C2 to C20 heterocyclic group substituted with at least one deuterium; or

at least one of R¹ to R⁵ may be, e.g., deuterium, a C6 to C30 aryl group substituted with at least one deuterium, or a C2 to C30 heterocyclic group substituted with at least one deuterium.

The compound represented by Chemical Formula 1 may have a structure of a basic skeleton in which triphenylene is substituted with a dibenzofuranyl group or a dibenzothiophenyl group and the basic skeleton is substituted with at least one deuterium.

As it is substituted with at least one deuterium, zero-point energy and vibration energy of the compound may be further lowered. Accordingly, the compound may have further lower energy in a ground state and may make a thin film formed therefrom into an amorphous state due to the weakened intermolecular interactions, further improving heat resistance and thus effectively improving life-span. In an implementation, when applied, an organic light emitting diode with high efficiency and particularly, a long life-span may be realized.

In an implementation, L¹ may be, e.g., a C6 to C20 arylene group substituted with at least one deuterium or a C2 to C20 heterocyclic group substituted with at least one deuterium and simultaneously at least one of R¹ to R⁵ may be, e.g., deuterium, a C6 to C30 aryl group substituted with at least one deuterium, or a C2 to C30 heterocyclic group substituted with at least one deuterium.

In an implementation, L¹ may be, e.g., a phenylene group substituted with at least one deuterium, a biphenylene group substituted with at least one deuterium, a terphenylene group substituted with at least one deuterium, a naphthylene group substituted with at least one deuterium, an anthracenylene group substituted with at least one deuterium, a phenanthrenylene group substituted with at least one deuterium, a triphenylenylene group substituted with at least one deuterium, a fluorenylene group substituted with at least one deuterium, a dibenzofuranylene group substituted with at least one deuterium, or a dibenzothiophenylene group substituted with at least one deuterium.

In an implementation, L¹ may be, e.g., a phenylene group substituted with at least one deuterium or a biphenylene group substituted with at least one deuterium.

In an implementation, at least one of R¹ to R⁵ may be, e.g., deuterium, a phenyl group substituted with at least one deuterium, a biphenyl group substituted with at least one deuterium, a terphenyl group substituted with at least one deuterium, a naphtyl group substituted with at least one deuterium, an anthracenyl group substituted with at least one deuterium, a phenanthrenyl group substituted with at least one deuterium, a triphenylene group substituted with at least one deuterium, a fluorenyl group substituted with at least one deuterium, a carbazolyl group substituted with at least one deuterium, a dibenzofuranyl group substituted with at least one deuterium, or a dibenzothiophenyl group substituted with at least one deuterium.

In an implementation, at least one of R¹ to R⁵ may be, e.g., deuterium, a phenyl group substituted with at least one deuterium, or a biphenyl group substituted with at least one deuterium.

In an implementation, the compound represented by Chemical Formula 1 may be, e.g., represented by one of Chemical Formula 1-1 to Chemical Formula 1-4, according to the specific substitution point of the dibenzofuranyl group or the dibenzothiophenyl group substituted for triphenylene.

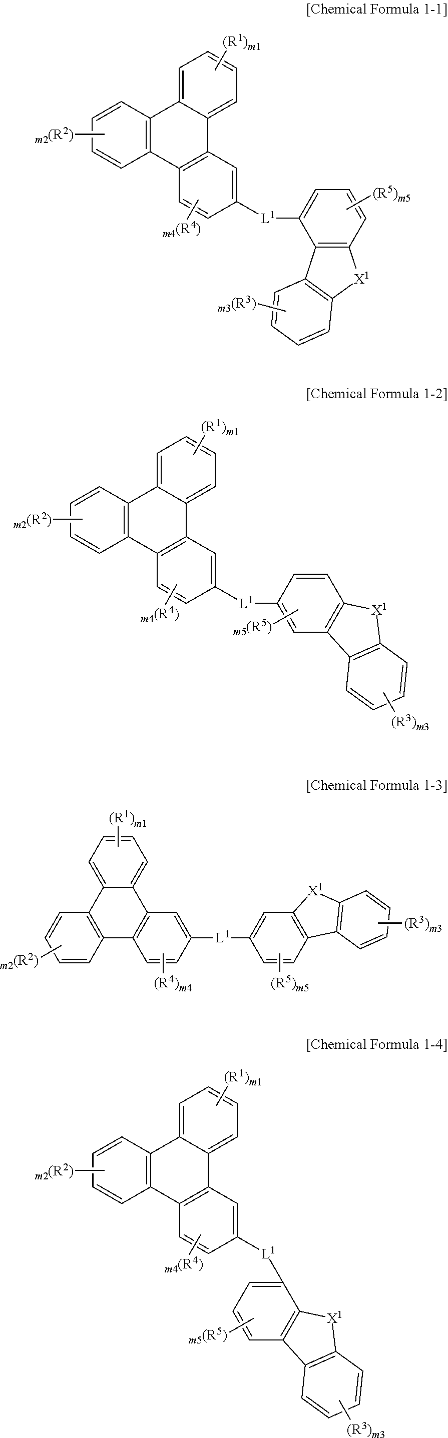

In Chemical Formula 1-1 to Chemical Formula 1-4,X¹, L¹, R¹ to R⁵, and m1 to m5 may be defined the same as those of Chemical Formula 1 described above.

In an implementation, the compound represented by Chemical Formula 1 may be represented by, e.g., Chemical Formula 1-1 or Chemical Formula 1-4.

In an implementation, L¹ in Chemical Formula 1-1 and Chemical Formula 1-4 may be, e.g., a phenylene group substituted with at least one deuterium or a biphenylene group substituted with at least one deuterium.

In an implementation, L¹ of Chemical Formula 1-1 and Chemical Formula 1-4 may be, e.g., a phenylene group in which all hydrogens are substituted with deuterium or a biphenylene group in which all hydrogens are substituted with deuterium, e.g., may be a linking group of Group I.

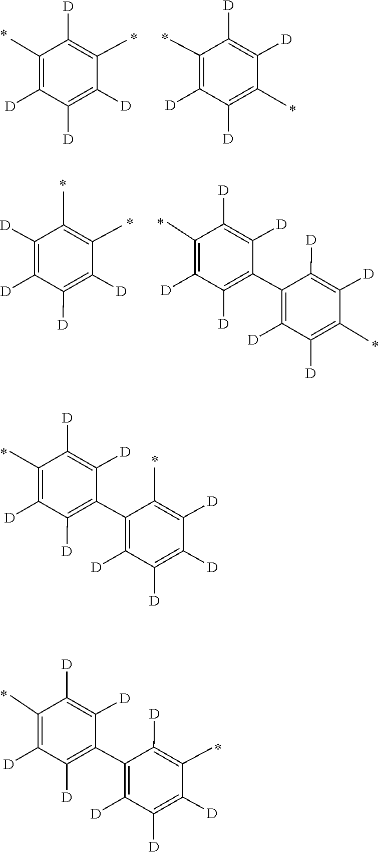

In an implementation, in Chemical Formula 1-1 and Chemical Formula 1-4, R¹ to R⁵ may be, e.g., hydrogen, deuterium, a substituted or unsubstituted phenyl group, or a substituted or unsubstituted biphenyl group, and at least one of R¹ to R⁵ may be, e.g., deuterium, a phenyl group substituted with at least one deuterium, or a biphenyl group substituted with at least one deuterium.

In an implementation, at least two of R¹ to R⁵ may be deuterium.

In an implementation, four R¹ may be deuterium.

In an implementation, three R¹ may be deuterium, and the remaining one may be a phenyl group substituted with at least one deuterium or a biphenyl group substituted with at least one deuterium.

In an implementation, four R² may be deuterium.

In an implementation, three R² may be deuterium, and the remaining one may be a phenyl group substituted with at least one deuterium or a biphenyl group substituted with at least one deuterium.

In an implementation, four R³ may be deuterium.

In an implementation, three R³ may be deuterium, and the remaining one may be a phenyl group substituted with at least one deuterium or a biphenyl group substituted with at least one deuterium.

In an implementation, three R⁴ may be deuterium.

In an implementation, two R⁴ may be deuterium, and the remaining one may be a phenyl group substituted with at least one deuterium or a biphenyl group substituted with at least one deuterium.

In an implementation, three R⁵ may be deuterium.

In an implementation, two R⁵ may be deuterium, and the remaining one may be a phenyl group substituted with at least one deuterium or a biphenyl group substituted with at least one deuterium.

In an implementation, R¹ to R⁵ may each be deuterium, ml to m3 may each be 4, and m4 and m5 may each be 3.

In an implementation, L¹ may be a phenylene group substituted with at least one deuterium or a biphenylene group substituted with at least one deuterium, and at least one of R¹ to R⁵ may be a phenyl group substituted with at least one deuterium or a biphenyl group substituted with at least one deuterium, and the remainder may be deuterium.

In an implementation, L¹ may be a phenylene group all substituted with deuterium or a biphenylene group all substituted with deuterium, and R¹ to R⁵ may each independently be deuterium, a phenyl group all substituted with deuterium, or a biphenyl group substituted with all deuterium.

In an implementation, the compound for the organic optoelectronic device represented by Chemical Formula 1 may be, e.g., a compound of Group 1.

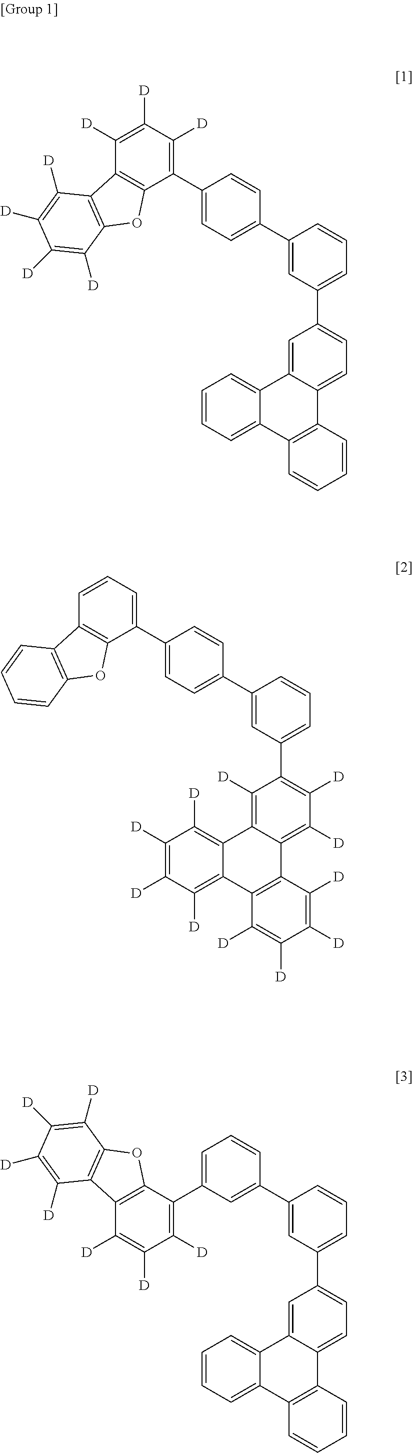 [SMILES: [2H]c1c([2H])c([2H])c2c(oc3c(-c4ccc(-c5cccc(-c6ccc7c8ccccc8c8ccccc8c7c6)c5)cc4)c([2H])c([2H])c([2H])c32)c1[2H] ; [2H]c1c([2H])c([2H])c2c(c1[2H])c1c([2H])c([2H])c([2H])c([2H])c1c1c([2H])c(-c3cccc(-c4ccc(-c5cccc6c5oc5ccccc56)cc4)c3)c([2H])c([2H])c21 ; [2H]c1c([2H])c([2H])c2c(oc3c(-c4cccc(-c5cccc(-c6ccc7c8ccccc8c8ccccc8c7c6)c5)c4)c([2H])c([2H])c([2H])c32)c1[2H]]

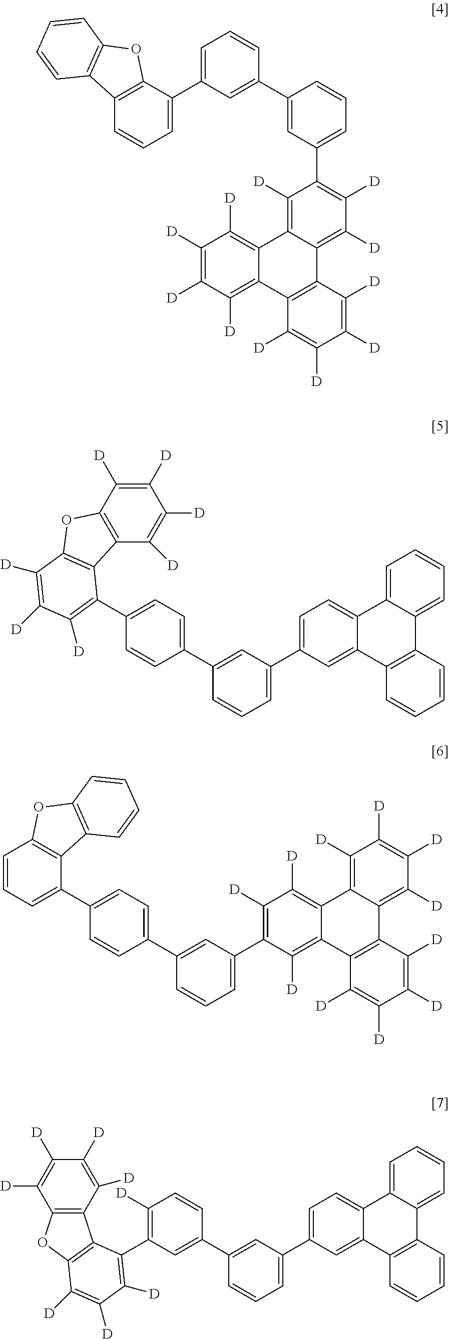

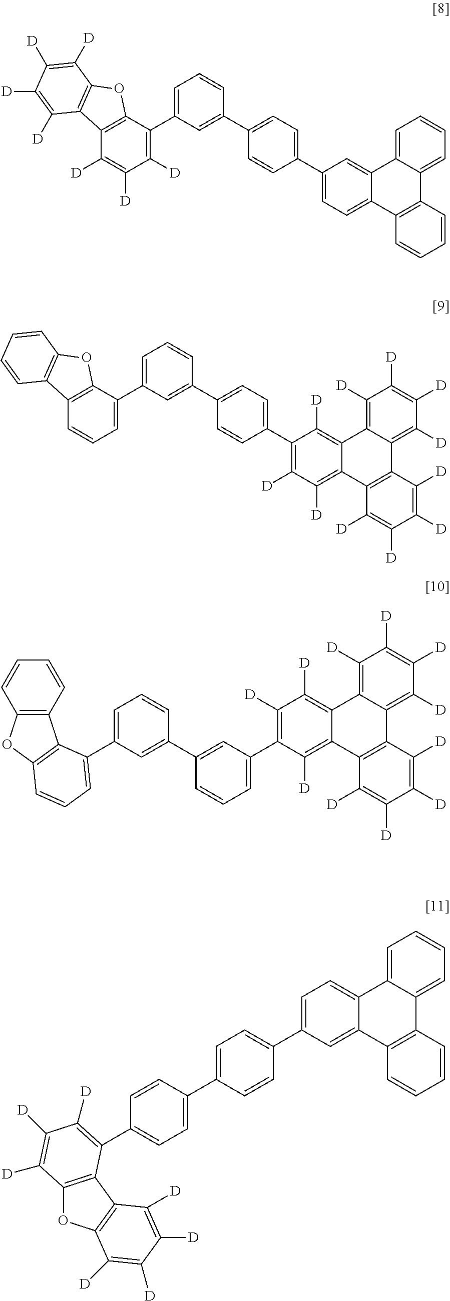 [SMILES: [2H]c1c([2H])c([2H])c2c(oc3c(-c4cccc(-c5ccc(-c6ccc7c8ccccc8c8ccccc8c7c6)cc5)c4)c([2H])c([2H])c([2H])c32)c1[2H] ; [2H]c1c([2H])c([2H])c2c(c1[2H])c1c([2H])c([2H])c([2H])c([2H])c1c1c([2H])c(-c3ccc(-c4cccc(-c5cccc6c5oc5ccccc56)c4)cc3)c([2H])c([2H])c21 ; [2H]c1c([2H])c([2H])c2c(c1[2H])c1c([2H])c([2H])c([2H])c([2H])c1c1c([2H])c(-c3cccc(-c4cccc(-c5cccc6oc7ccccc7c56)c4)c3)c([2H])c([2H])c21 ; [2H]c1c([2H])c([2H])c2c(oc3c([2H])c([2H])c([2H])c(-c4ccc(-c5ccc(-c6ccc7c8ccccc8c8ccccc8c7c6)cc5)cc4)c32)c1[2H]]

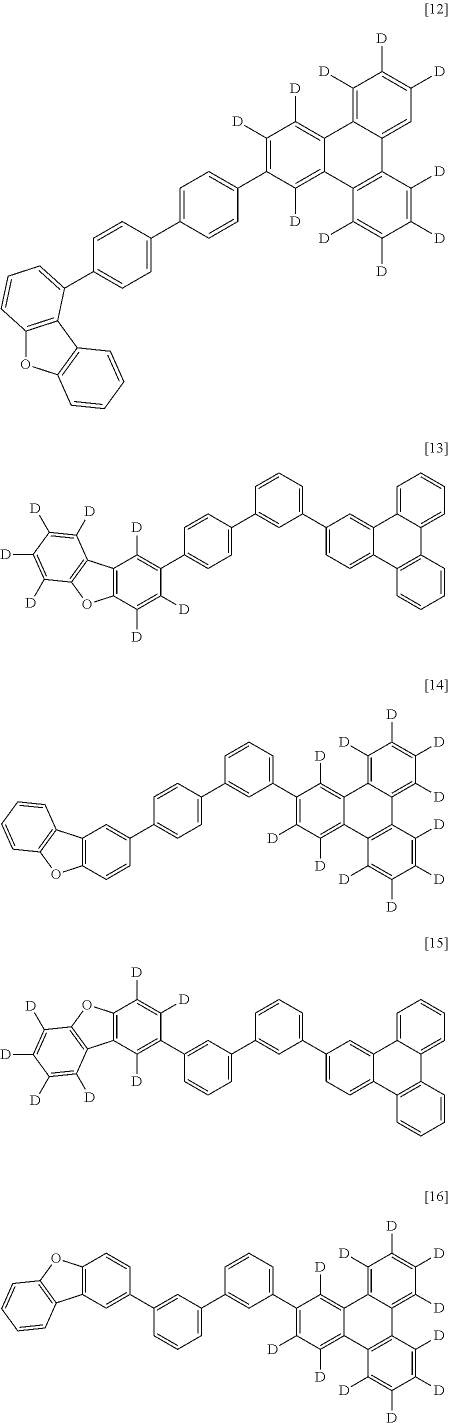

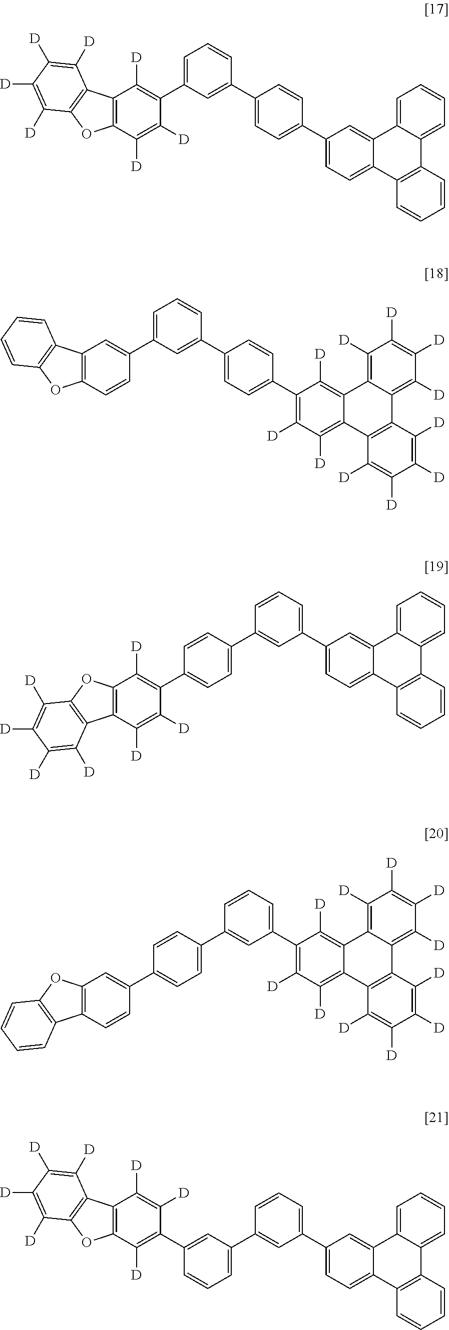

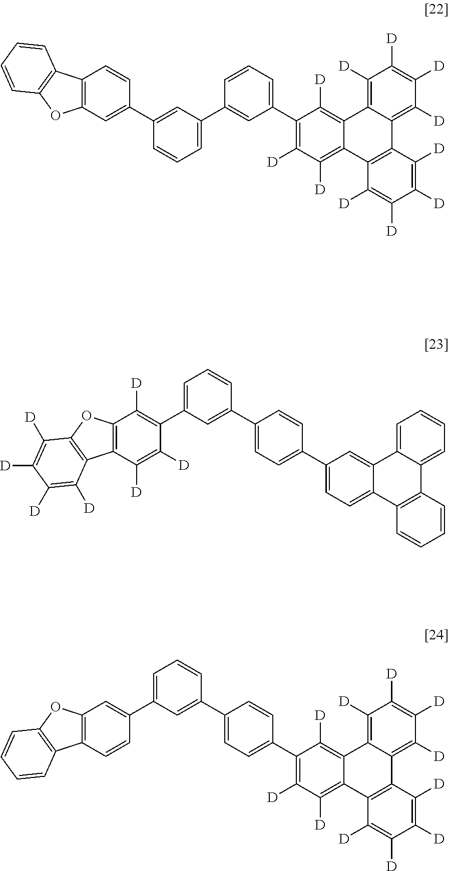 [SMILES: [2H]c1c([2H])c([2H])c2c(c1[2H])c1c([2H])c([2H])c([2H])c([2H])c1c1c([2H])c(-c3cccc(-c4cccc(-c5ccc6c(c5)oc5ccccc56)c4)c3)c([2H])c([2H])c21 ; [2H]c1c([2H])c([2H])c2c(oc3c([2H])c(-c4cccc(-c5ccc(-c6ccc7c8ccccc8c8ccccc8c7c6)cc5)c4)c([2H])c([2H])c32)c1[2H] ; [2H]c1c([2H])c([2H])c2c(c1[2H])c1c([2H])c([2H])c([2H])c([2H])c1c1c([2H])c(-c3ccc(-c4cccc(-c5ccc6c(c5)oc5ccccc56)c4)cc3)c([2H])c([2H])c21]

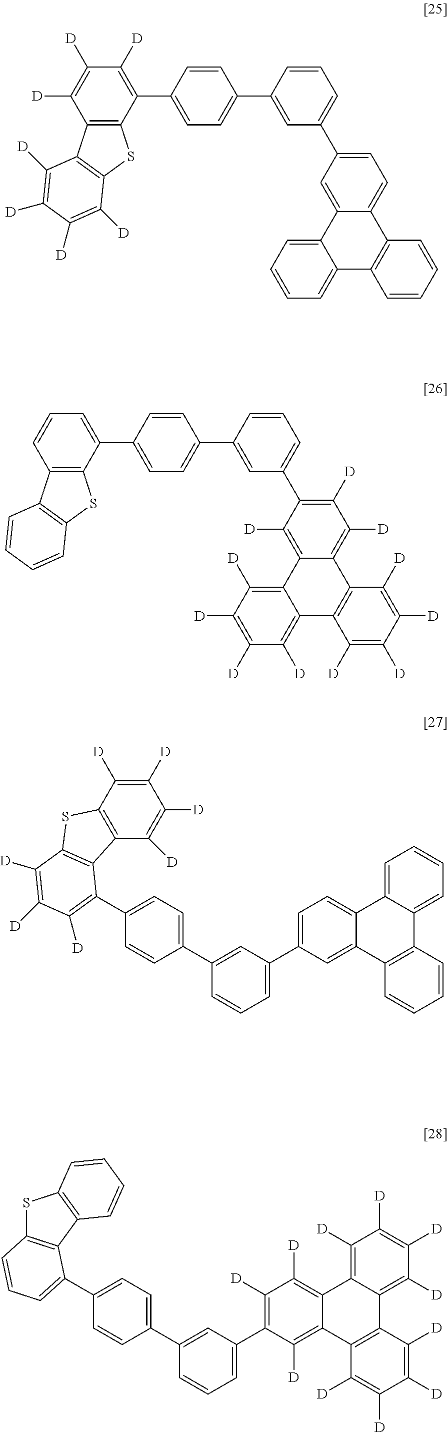 [SMILES: [2H]c1c([2H])c([2H])c2c(sc3c(-c4ccc(-c5cccc(-c6ccc7c8ccccc8c8ccccc8c7c6)c5)cc4)c([2H])c([2H])c([2H])c32)c1[2H] ; [2H]c1c([2H])c([2H])c2c(c1[2H])c1c([2H])c([2H])c([2H])c([2H])c1c1c([2H])c(-c3cccc(-c4ccc(-c5cccc6c5sc5ccccc56)cc4)c3)c([2H])c([2H])c21 ; [2H]c1c([2H])c([2H])c2c(sc3c([2H])c([2H])c([2H])c(-c4ccc(-c5cccc(-c6ccc7c8ccccc8c8ccccc8c7c6)c5)cc4)c32)c1[2H] ; [2H]c1c([2H])c([2H])c2c(c1[2H])c1c([2H])c([2H])c([2H])c([2H])c1c1c([2H])c(-c3cccc(-c4ccc(-c5cccc6sc7ccccc7c56)cc4)c3)c([2H])c([2H])c21]

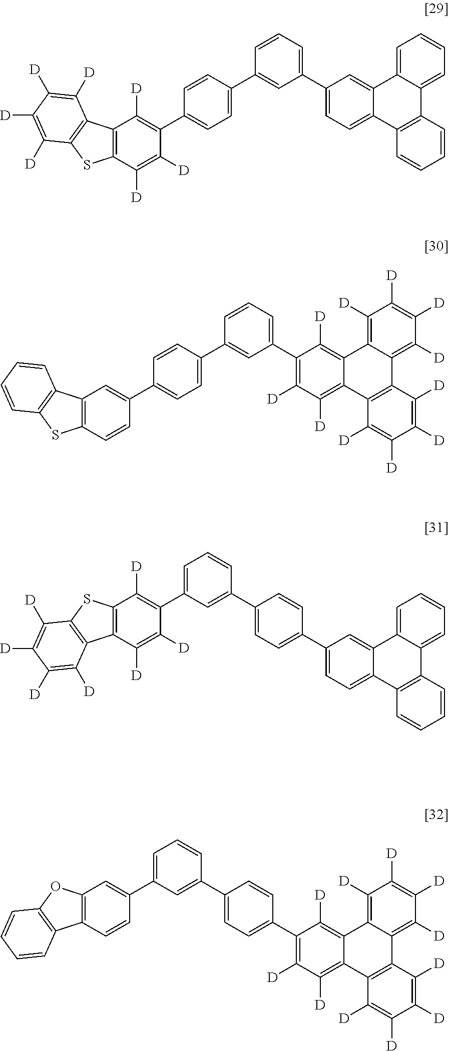 [SMILES: [2H]c1c([2H])c([2H])c2c(sc3c([2H])c([2H])c(-c4ccc(-c5cccc(-c6ccc7c8ccccc8c8ccccc8c7c6)c5)cc4)c([2H])c32)c1[2H] ; [2H]c1c([2H])c([2H])c2c(c1[2H])c1c([2H])c([2H])c([2H])c([2H])c1c1c([2H])c(-c3cccc(-c4ccc(-c5ccc6sc7ccccc7c6c5)cc4)c3)c([2H])c([2H])c21 ; [2H]c1c([2H])c([2H])c2c(sc3c([2H])c(-c4cccc(-c5ccc(-c6ccc7c8ccccc8c8ccccc8c7c6)cc5)c4)c([2H])c([2H])c32)c1[2H] ; [2H]c1c([2H])c([2H])c2c(c1[2H])c1c([2H])c([2H])c([2H])c([2H])c1c1c([2H])c(-c3ccc(-c4cccc(-c5ccc6c(c5)oc5ccccc56)c4)cc3)c([2H])c([2H])c21]

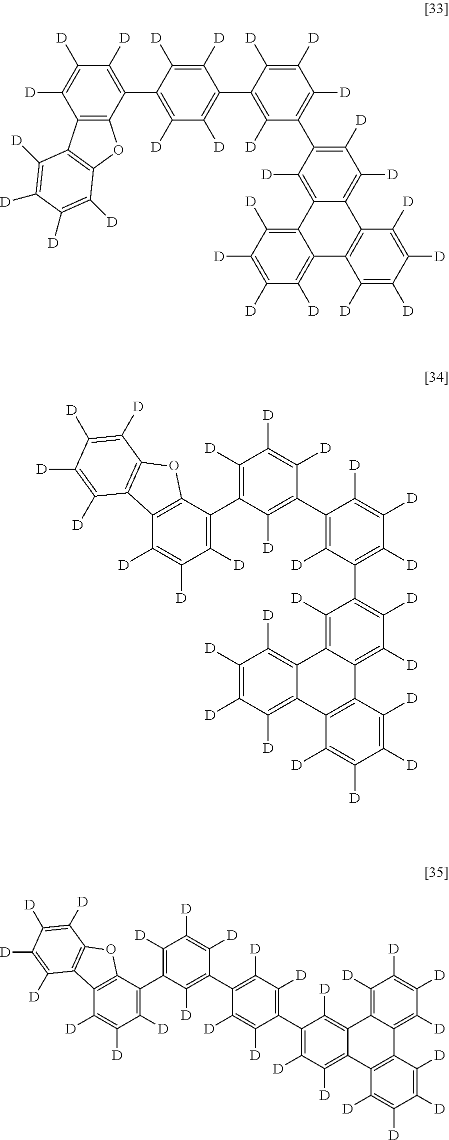

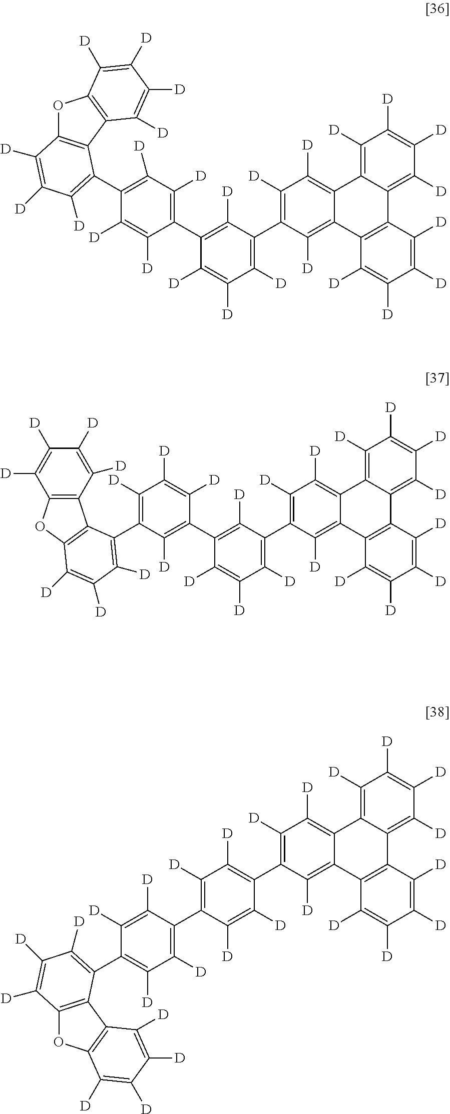

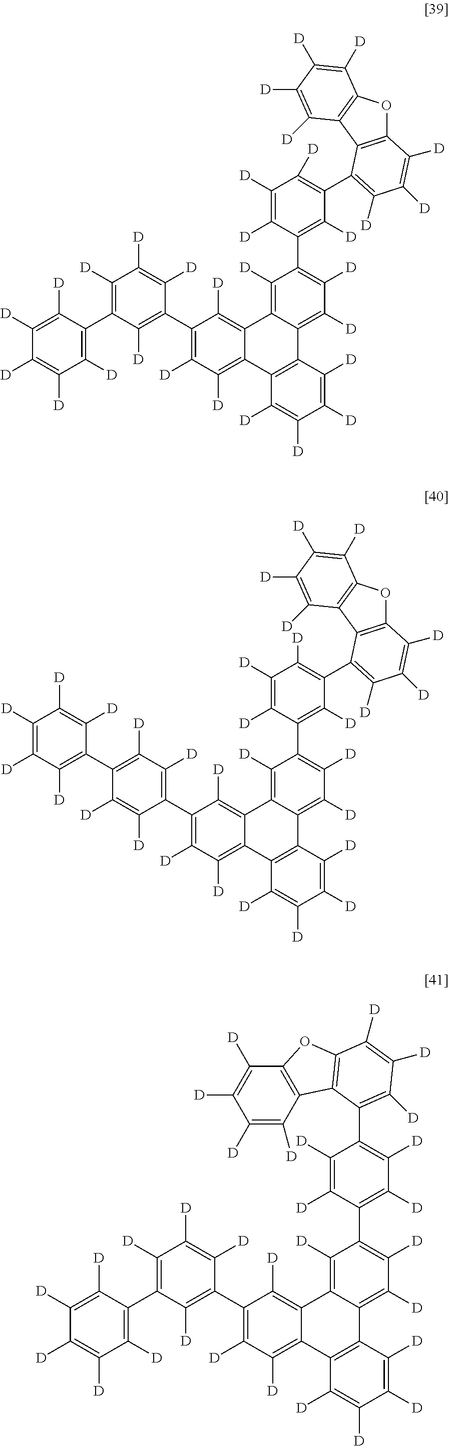

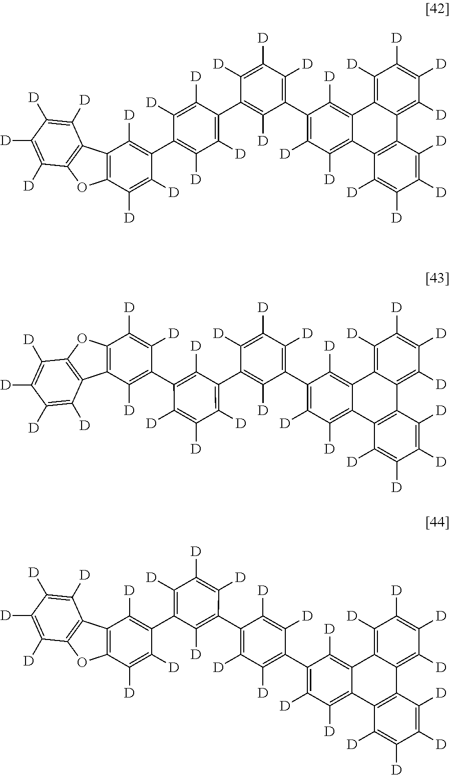

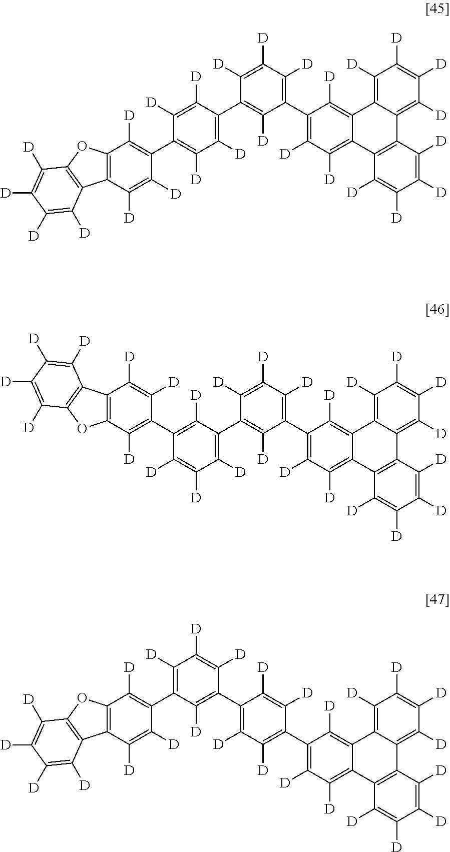

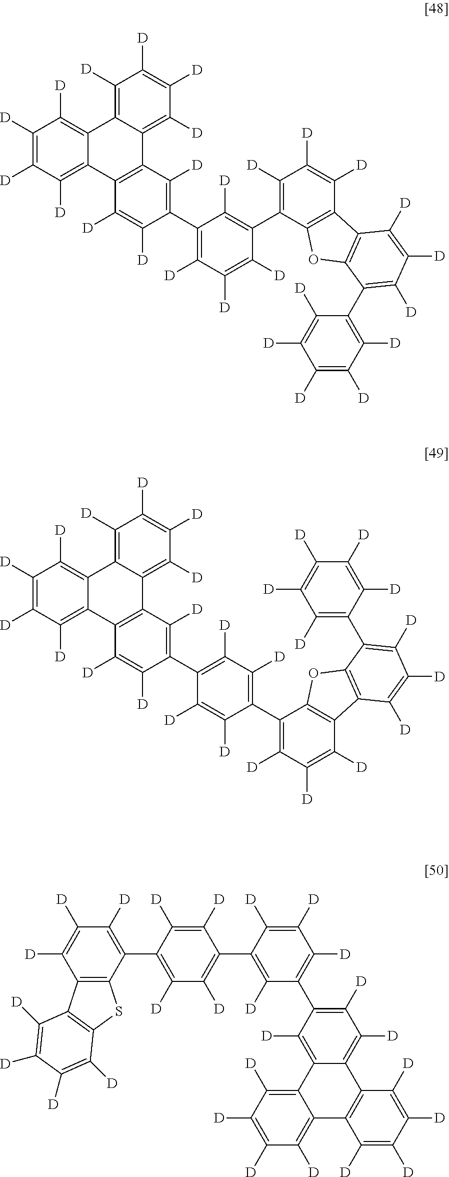

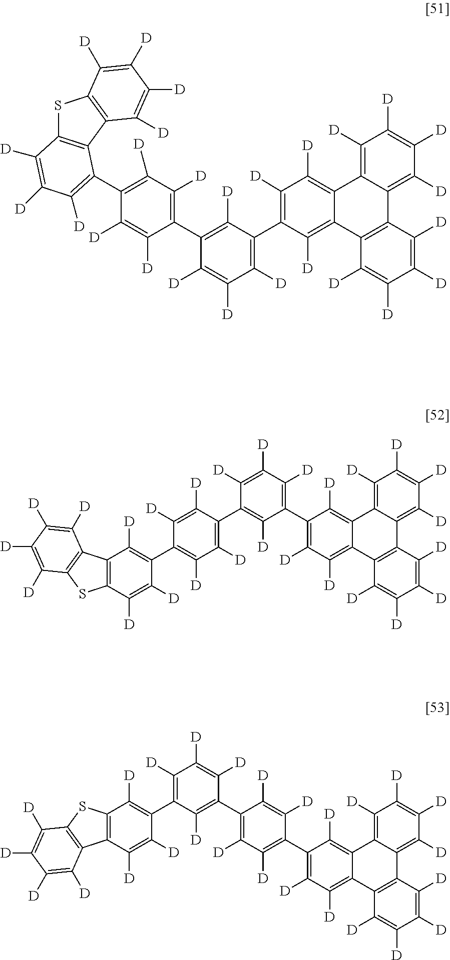

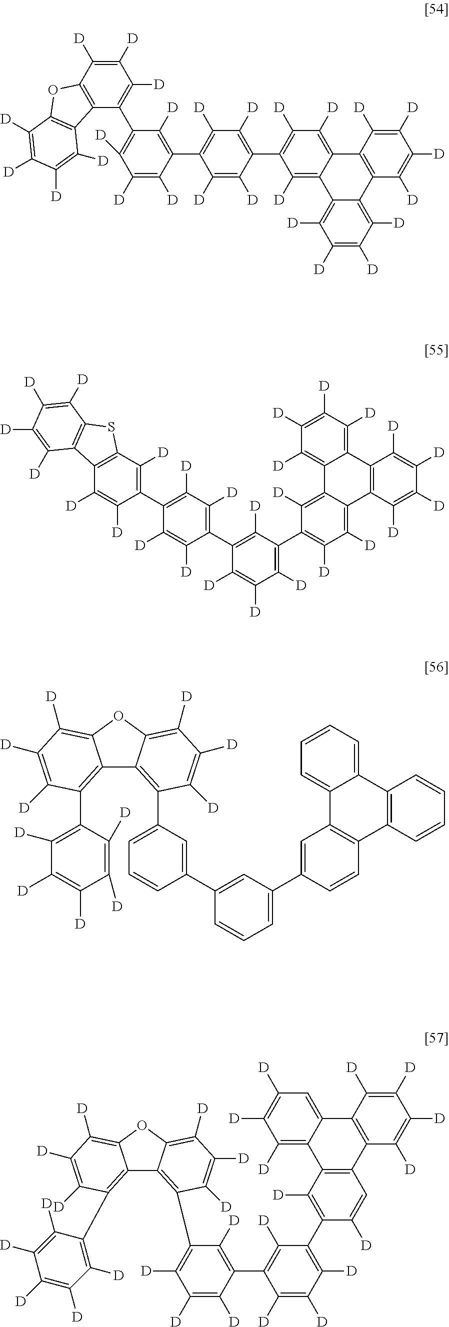

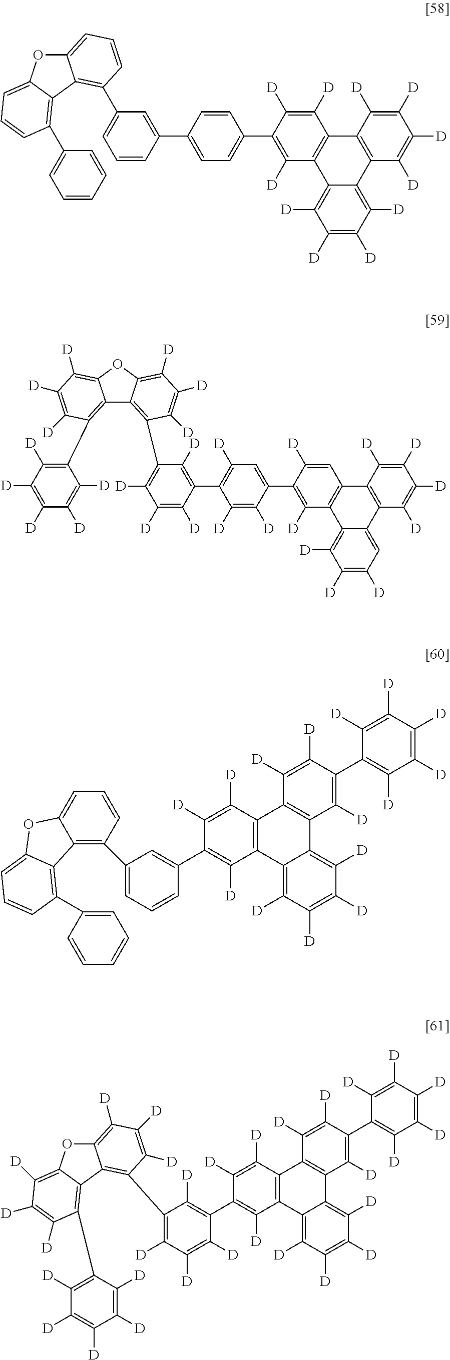

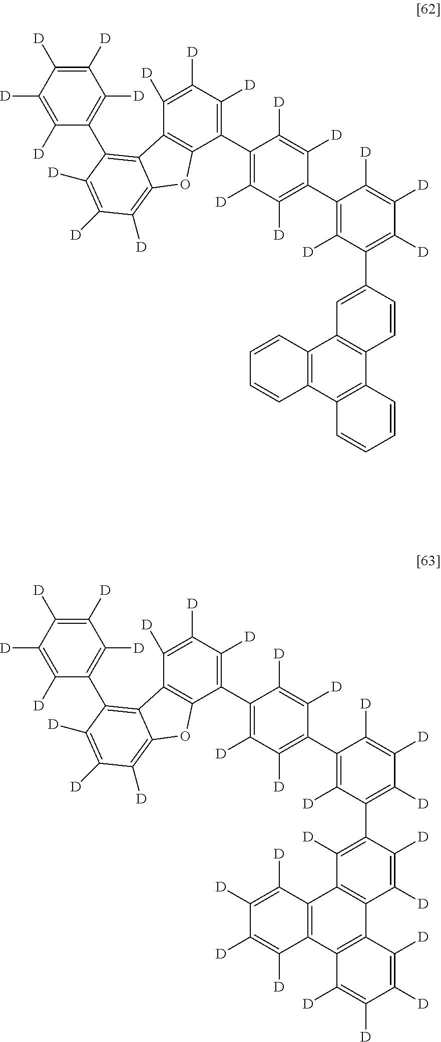 [SMILES: [2H]c1c([2H])c([2H])c(-c2c([2H])c([2H])c([2H])c3oc4c(-c5c([2H])c([2H])c(-c6c([2H])c([2H])c([2H])c(-c7ccc8c9ccccc9c9ccccc9c8c7)c6[2H])c([2H])c5[2H])c([2H])c([2H])c([2H])c4c23)c([2H])c1[2H] ; [2H]c1c([2H])c([2H])c(-c2c([2H])c([2H])c([2H])c3oc4c(-c5c([2H])c([2H])c(-c6c([2H])c([2H])c([2H])c(-c7c([2H])c([2H])c8c9c([2H])c([2H])c([2H])c([2H])c9c9c([2H])c([2H])c([2H])c([2H])c9c8c7[2H])c6[2H])c([2H])c5[2H])c([2H])c([2H])c([2H])c4c23)c([2H])c1[2H]]

One or more compounds may be further included in addition to the aforementioned compound for the organic optoelectronic device.

In an implementation, the aforementioned compound for the organic optoelectronic device may be applied in a form of a composition further including a suitable host material.

In an implementation, the aforementioned compound for the organic optoelectronic device may further include a dopant.

In an implementation, the dopant may be a phosphorescent dopant, e.g., a red or green phosphorescent dopant.

A dopant is a material that emits light by being mixed in a small amount with the compound for the organic optoelectronic device. In general, the dopant may be a material such as a metal complex that emits light by multiple excitation into a triplet or more. The dopant may be, e.g., an inorganic, organic, or organic-inorganic compound, and may include one or two or more.

An example of the dopant may be a phosphorescent dopant, and examples of the phosphorescent dopant may include an organometallic compound including Ir, Pt, Os, Ti, Zr, Hf, Eu, Tb, Tm, Fe, Co, Ni, Ru, Rh, Pd, or a combination thereof. In an implementation, the phosphorescent dopant may include, e.g., a compound represented by Chemical Formula Z.

L²MX² [Chemical Formula Z]

In Chemical Formula Z, M may be a metal, and L² and X² may each independently be ligands forming a complex with M.

M may be, e.g., Ir, Pt, Os, Ti, Zr, Hf, Eu, Tb, Tm, Fe, Co, Ni, Ru, Rh, Pd, or a combination thereof, and L² and X² may be, e.g., a bidentate ligand.

Examples of the ligands represented by L² and X² may be selected from the chemical formulas of Group A.

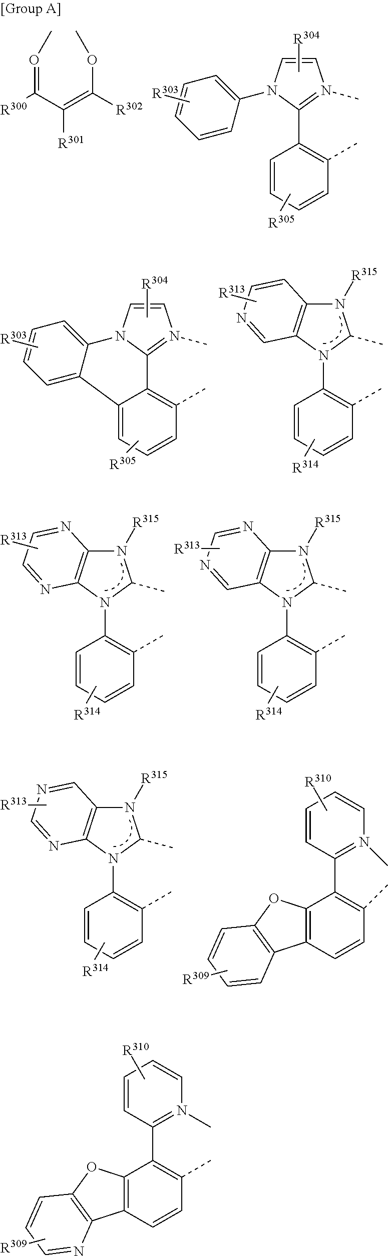

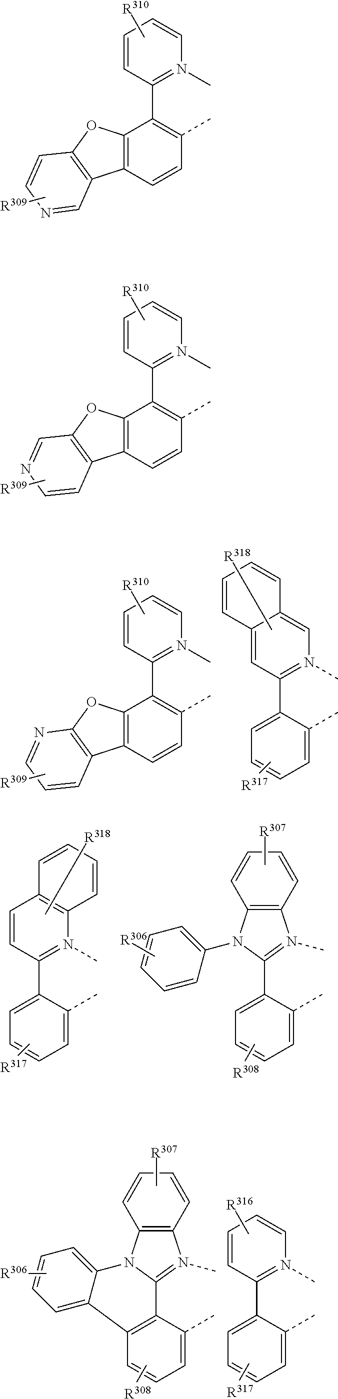

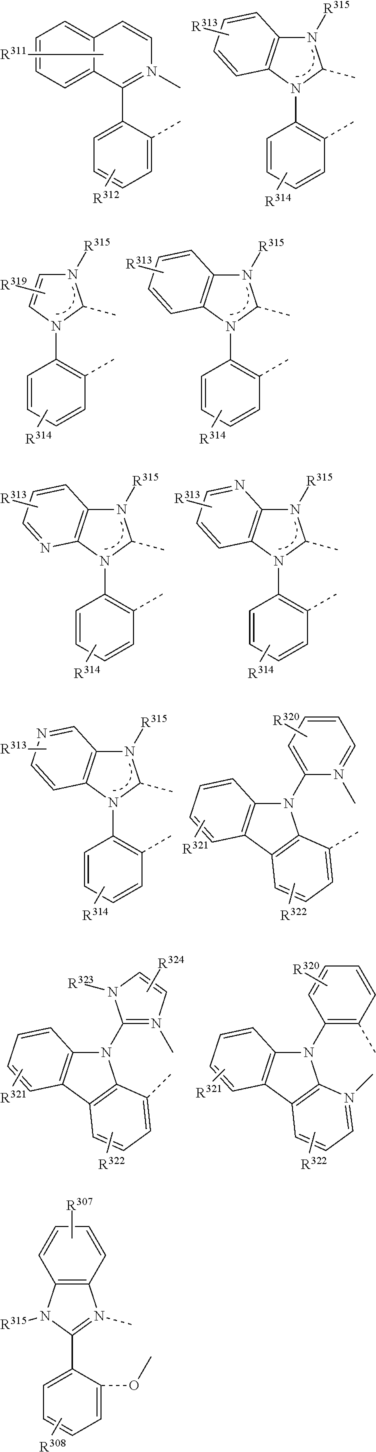

In Group A,

R³⁰⁰ to R³⁰² may each independently be, e.g., hydrogen, deuterium a C1 to C30 alkyl group that is substituted or unsubstituted with a halogen, a C6 to C30 aryl group that is substituted or unsubstituted with a C1 to C30 alkyl, or a halogen, and

R³⁰³ to R³²⁴ may each independently be, e.g., hydrogen, deuterium, a halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and C6 to C30 aryl group, or a triarylsilyl group having a substituted or unsubstituted C6 to C30 aryl group.

In an implementation, a dopant represented by Chemical Formula II may be included.

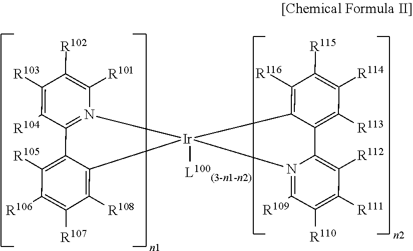

In Chemical Formula II,

R¹⁰¹ to R¹¹⁶ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴,

R¹³² to R¹³⁴ may each independently be, e.g., a C1 to C6 alkyl group,

at least one of R¹⁰¹ to R¹¹⁶ may be, e.g., a functional group represented by

Chemical Formula II-1,

L¹⁰⁰ may be, e.g., a bidentate ligand of a monovalent anion, and is a ligand that coordinates to iridium through a lone pair of carbons or heteroatoms, and

n1 and n2 may each independently be, e.g., an integer of 0 to 3 and n1+n2 may be an integer of 1 to 3.

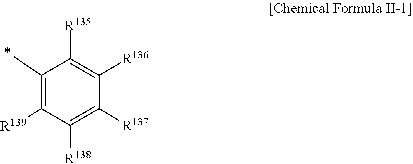

In Chemical Formula II-1,

R¹³⁵ to R¹³⁹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴, and

* indicates a portion linked to a carbon atom.

In an implementation, a dopant represented by Chemical Formula Z-1 may be included.

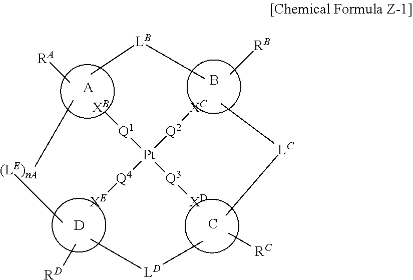

In Chemical Formula Z-1, rings A, B, C, and D may each independently be, e.g., a 5-membered or 6-membered carbocyclic or heterocyclic ring;

R^{A}, R^{B}, R^{C}, and R^{D} may each independently be, e.g., mono-, di-, tri-, or tetra-substitution, or unsubstitution;

L^{B}, L^{C}, and L^{D} may each independently be, e.g., a direct bond, BR, NR, PR, O, S, Se, C═O, S═O, SO₂, CRR′, SiRR′, GeRR′, or a combination thereof.

when nA is 1, L^{E} may be a direct bond, BR, NR, PR, O, S, Se, C═O, S═O, SO₂,

CRR′, SiRR′, GeRR′, or a combination thereof; and when nA is 0, L^{E} does not exist.

R^{A}, R^{B}, R^{C}, R^{D}, R, and R′ may each independently be, e.g., hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof; any adjacent R^{A}, R^{B}, R^{C}, R^{D}, R, and R′ are optionally linked to each other to provide a ring; X^{B}, X^{C}, X^{D}, and X^{E} are each independently selected from carbon and nitrogen; and Q¹, Q², Q³, and Q⁴ each represent oxygen or a direct bond.

The dopant according to an embodiment may be a platinum complex, and may be, e.g., represented by Chemical Formula III.

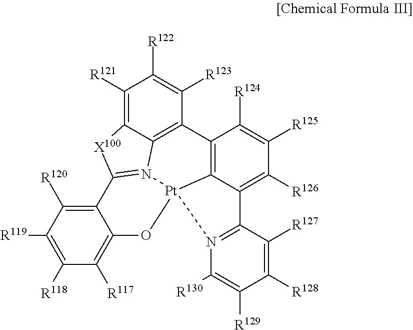

In Chemical Formula III,

X¹⁰⁰ may be, e.g., O, S, or NR¹³¹,

R¹¹⁷ to R¹³¹ may each independently be, e.g., hydrogen, deuterium, a substituted or unsubstituted C1 to C10 alkyl group, a substituted or unsubstituted C6 to C20 aryl group or —SiR¹³²R¹³³R¹³⁴,

R¹³² to R¹³⁴ may each independently be, e.g., a C1 to C6 alkyl group, and

at least one of R¹¹⁷ to R¹³¹ may be, e.g., —SiR¹³²R¹³³R¹³⁴ or a tert-butyl group.

Hereinafter, an organic optoelectronic device including the aforementioned compound for the organic optoelectronic device is described.

The organic optoelectronic device may be a suitable device to convert electrical energy into photoenergy and vice versa, e.g., an organic photoelectric device, an organic light emitting diode, an organic solar cell, or an organic photoconductor drum.

Herein, an organic light emitting diode as one example of an organic optoelectronic device is described referring to drawings.

The FIGURE is a cross-sectional view illustrating an organic light emitting diode according to an embodiment.

Referring to the FIGURE, an organic light emitting diode 100 according to an embodiment may include, e.g., an anode 120 and a cathode 110 facing each other and an organic layer 105 between the anode 120 and cathode 110.

The anode 120 may be made of a conductor having a large work function to help hole injection, and may be, e.g., a metal, a metal oxide, or a conductive polymer. The anode 120 may be, e.g., a metal such as nickel, platinum, vanadium, chromium, copper, zinc, gold, and the like or an alloy thereof; a metal oxide such as zinc oxide, indium oxide, indium tin oxide (ITO), indium zinc oxide (IZO), and the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, or polyaniline.

The cathode 110 may be made of a conductor having a small work function to help electron injection, and may be, e.g., a metal, a metal oxide, or a conductive polymer. The cathode 110 may be, e.g., a metal such as magnesium, calcium, sodium, potassium, titanium, indium, yttrium, lithium, gadolinium, aluminum silver, tin, lead, cesium, barium, or the like, or an alloy thereof; or a multi-layer structure material such as LiF/Al, LiO₂/Al, LiF/Ca, or BaF₂/Ca.

The organic layer 105 may include the aforementioned compound for an organic optoelectronic device.

The organic layer 105 may include, e.g., the light emitting layer 130, and the light emitting layer 130 may include, e.g., the aforementioned compound for an organic optoelectronic device.

The composition for an organic optoelectronic device further including a dopant may be, e.g., a red light-emitting composition.

The light emitting layer 130 may include, e.g., the aforementioned compound for an organic optoelectronic device as a phosphorescent host.

The organic layer may further include a charge transport region in addition to the light emitting layer.

The charge transport region may be, e.g., the hole transport region 140.

The hole transport region 140 may help further increase hole injection and/or hole mobility between the anode 120 and the light emitting layer 130 and block electrons.

In an implementation, the hole transport region 140 may include a hole transport layer between the anode 120 and the light emitting layer 130, and a hole transport auxiliary layer between the light emitting layer 130 and the hole transport layer, and at least one of the compounds of Group B may be included in at least one of the hole transport layer and the hole transport auxiliary layer.

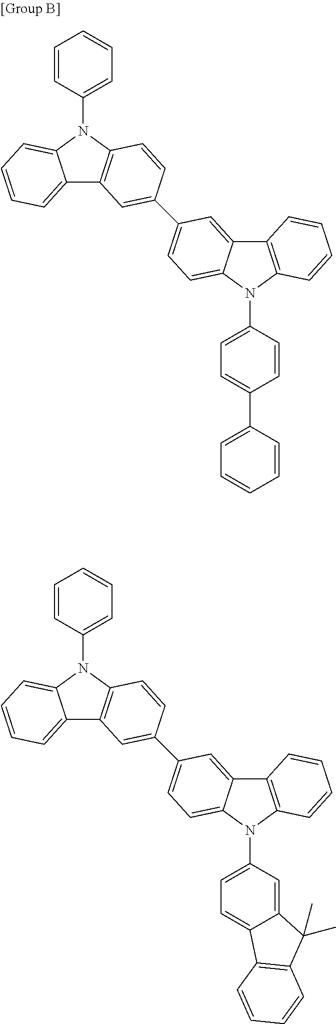 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21]

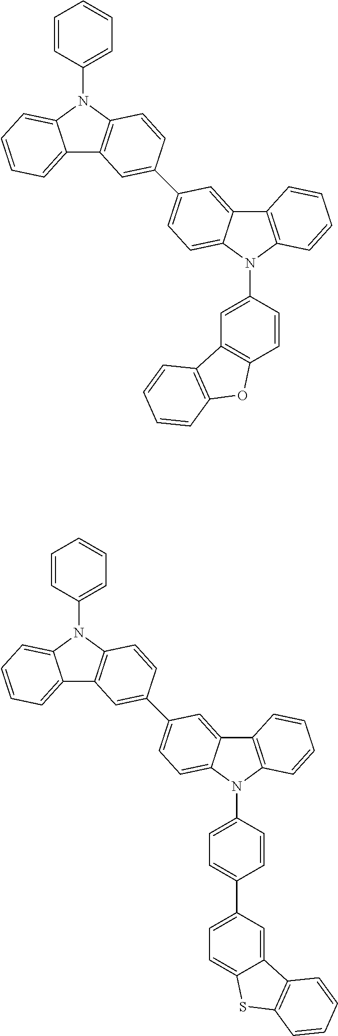 [SMILES: c1ccc(-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4ccc(-c5ccc6sc7ccccc7c6c5)cc4)ccc32)cc1]

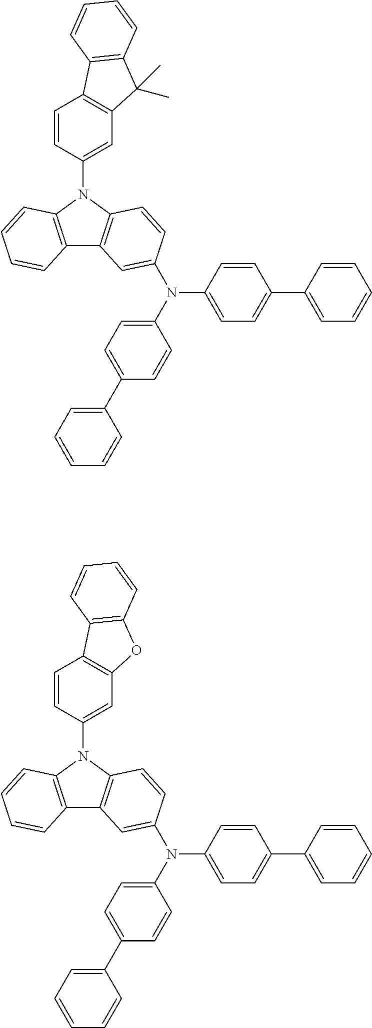 [SMILES: CC1(C)c2ccccc2-c2ccc(-n3c4ccccc4c4cc(N(c5ccc(-c6ccccc6)cc5)c5ccc(-c6ccccc6)cc5)ccc43)cc21 ; c1ccc(-c2ccc(N(c3ccc(-c4ccccc4)cc3)c3ccc4c(c3)c3ccccc3n4-c3ccc4c(c3)oc3ccccc34)cc2)cc1]

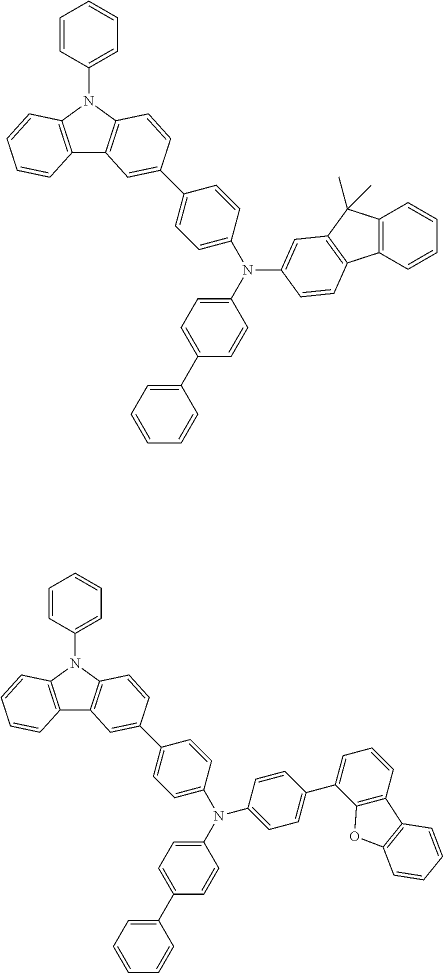 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1]

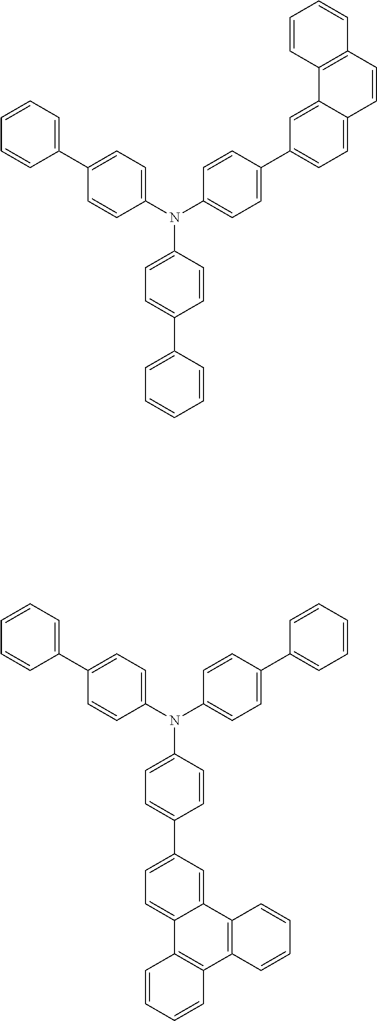 [SMILES: c1ccc(-c2ccc(N(c3ccc(-c4ccccc4)cc3)c3ccc(-c4ccc5ccc6ccccc6c5c4)cc3)cc2)cc1 ; c1ccc(-c2ccc(N(c3ccc(-c4ccccc4)cc3)c3ccc(-c4ccc5c6ccccc6c6ccccc6c5c4)cc3)cc2)cc1]

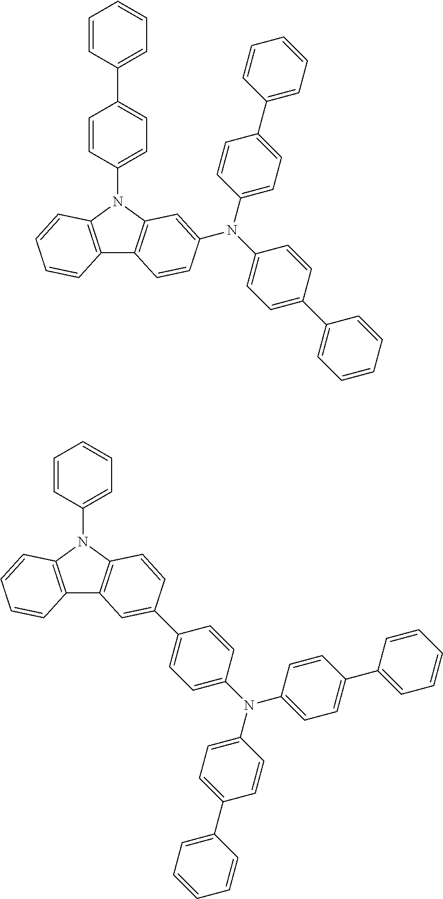 [SMILES: c1ccc(-c2ccc(N(c3ccc(-c4ccccc4)cc3)c3ccc4c5ccccc5n(-c5ccc(-c6ccccc6)cc5)c4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1]

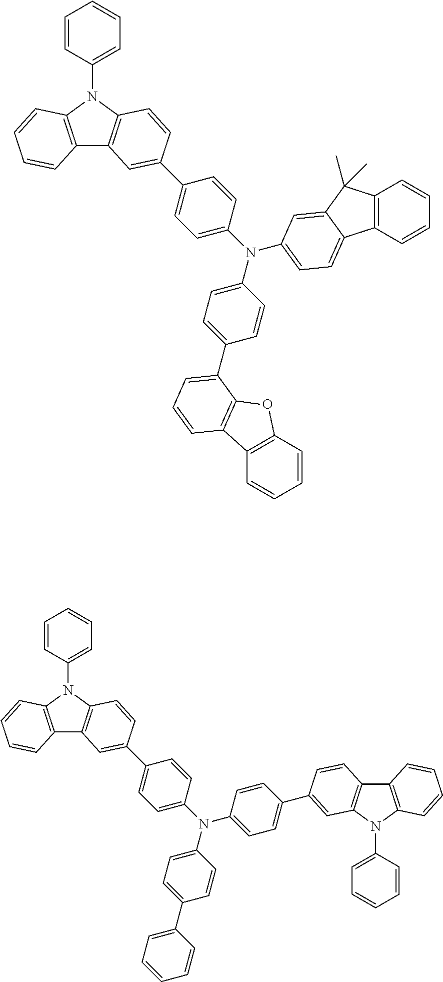 [SMILES: CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21 ; c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1]

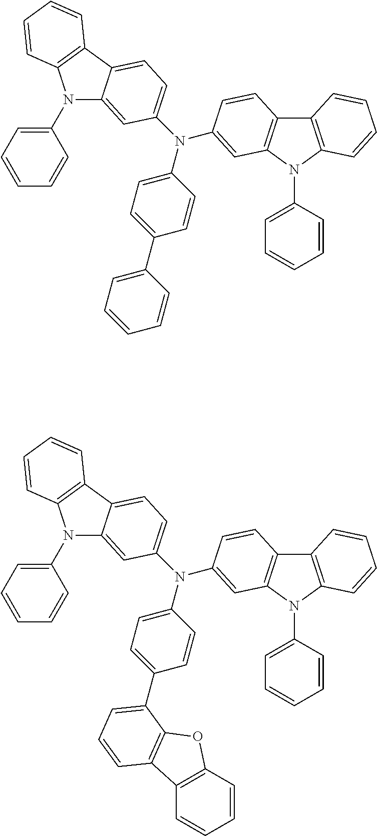 [SMILES: c1ccc(-c2ccc(N(c3ccc4c5ccccc5n(-c5ccccc5)c4c3)c3ccc4c5ccccc5n(-c5ccccc5)c4c3)cc2)cc1 ; c1ccc(-n2c3ccccc3c3ccc(N(c4ccc(-c5cccc6c5oc5ccccc56)cc4)c4ccc5c6ccccc6n(-c6ccccc6)c5c4)cc32)cc1]

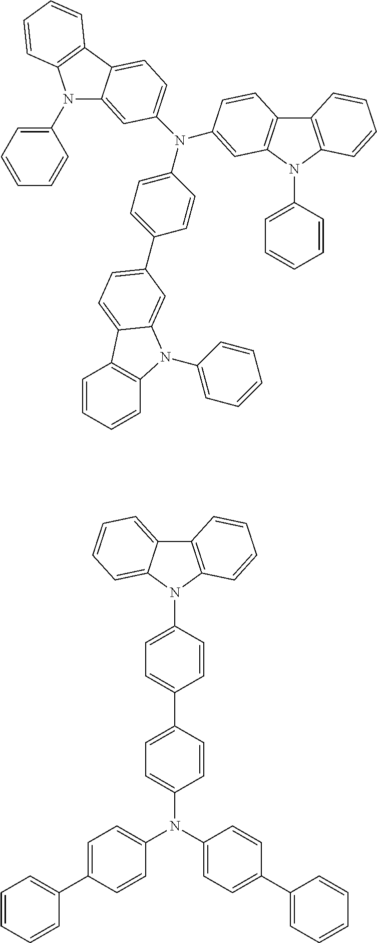 [SMILES: c1ccc(-n2c3ccccc3c3ccc(-c4ccc(N(c5ccc6c7ccccc7n(-c7ccccc7)c6c5)c5ccc6c7ccccc7n(-c7ccccc7)c6c5)cc4)cc32)cc1 ; c1ccc(-c2ccc(N(c3ccc(-c4ccccc4)cc3)c3ccc(-c4ccc(-n5c6ccccc6c6ccccc65)cc4)cc3)cc2)cc1]

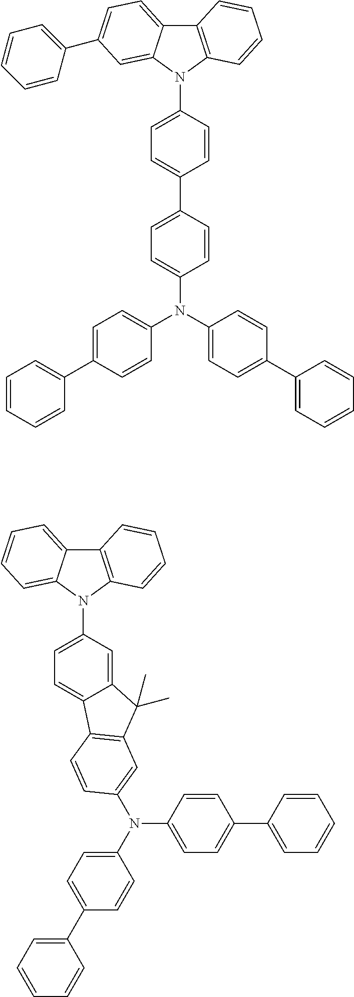 [SMILES: c1ccc(-c2ccc(N(c3ccc(-c4ccccc4)cc3)c3ccc(-c4ccc(-n5c6ccccc6c6ccc(-c7ccccc7)cc65)cc4)cc3)cc2)cc1 ; CC1(C)c2cc(N(c3ccc(-c4ccccc4)cc3)c3ccc(-c4ccccc4)cc3)ccc2-c2ccc(-n3c4ccccc4c4ccccc43)cc21]

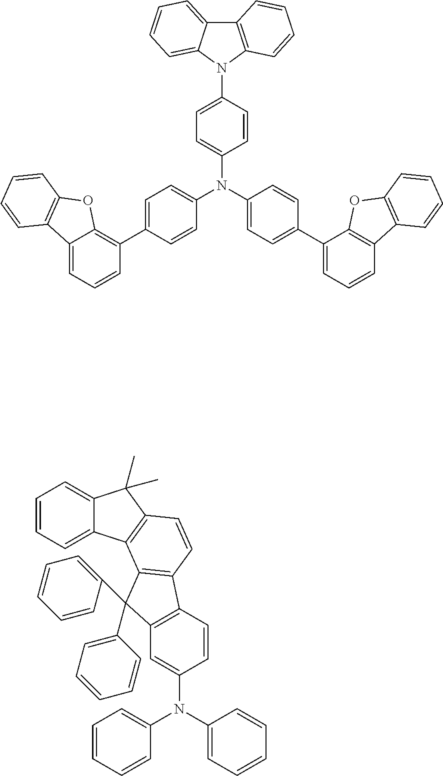 [SMILES: c1ccc2c(c1)oc1c(-c3ccc(N(c4ccc(-c5cccc6c5oc5ccccc56)cc4)c4ccc(-n5c6ccccc6c6ccccc65)cc4)cc3)cccc12 ; CC1(C)c2ccccc2-c2c1ccc1c2C(c2ccccc2)(c2ccccc2)c2cc(N(c3ccccc3)c3ccccc3)ccc2-1]

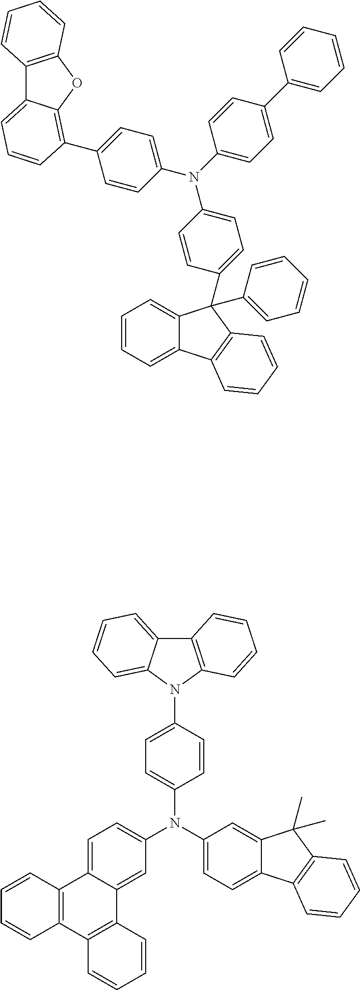 [SMILES: c1ccc(-c2ccc(N(c3ccc(-c4cccc5c4oc4ccccc45)cc3)c3ccc(C4(c5ccccc5)c5ccccc5-c5ccccc54)cc3)cc2)cc1 ; CC1(C)c2ccccc2-c2ccc(N(c3ccc(-n4c5ccccc5c5ccccc54)cc3)c3ccc4c5ccccc5c5ccccc5c4c3)cc21]

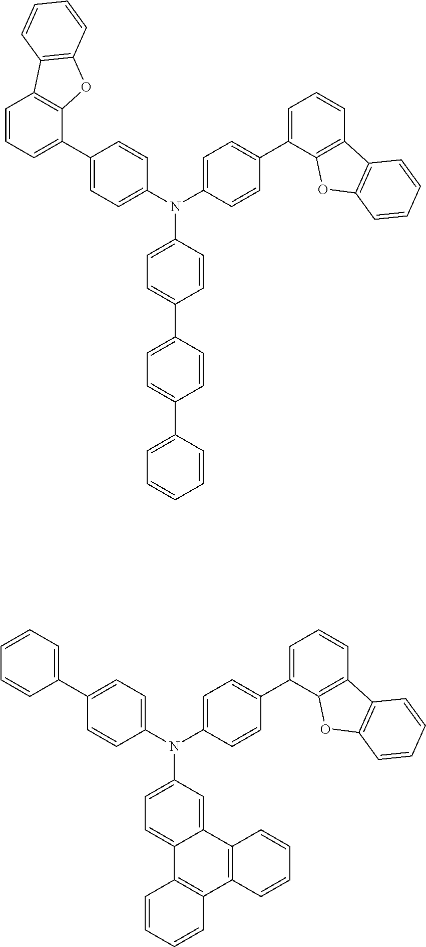 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(N(c3ccc(-c4cccc5c4oc4ccccc45)cc3)c3ccc4c5ccccc5c5ccccc5c4c3)cc2)cc1]

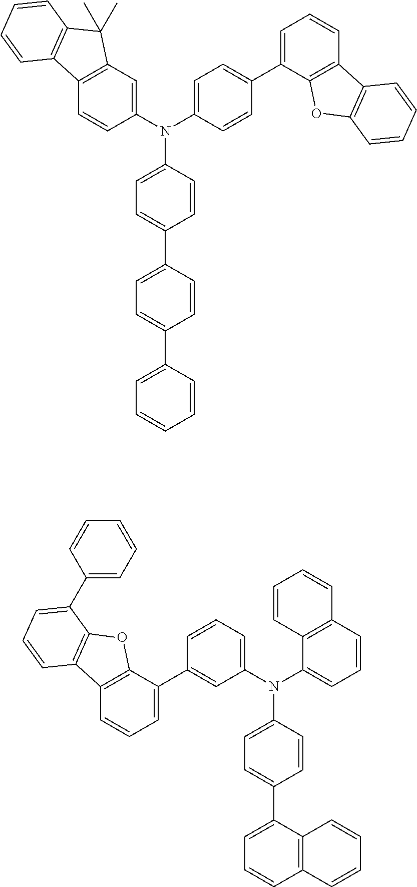 [SMILES: CC1(C)c2ccccc2-c2ccc(N(c3ccc(-c4ccc(-c5ccccc5)cc4)cc3)c3ccc(-c4cccc5c4oc4ccccc45)cc3)cc21 ; c1ccc(-c2cccc3c2oc2c(-c4cccc(N(c5ccc(-c6cccc7ccccc67)cc5)c5cccc6ccccc56)c4)cccc23)cc1]

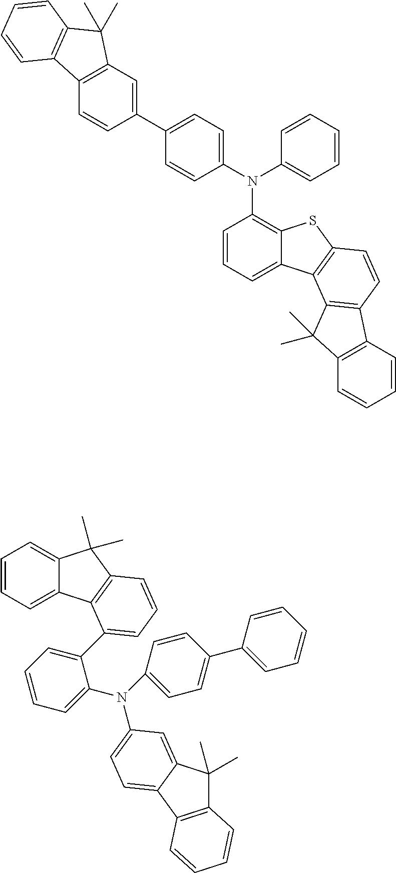 [SMILES: CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21]

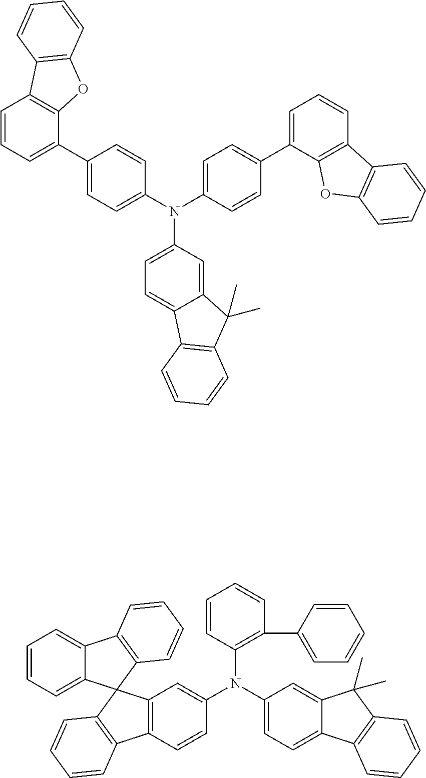 [SMILES: CC1(C)c2ccccc2-c2ccc(N(c3ccc(-c4cccc5c4oc4ccccc45)cc3)c3ccc(-c4cccc5c4oc4ccccc45)cc3)cc21 ; CC1(C)c2ccccc2-c2ccc(N(c3ccc4c(c3)C3(c5ccccc5-c5ccccc53)c3ccccc3-4)c3ccccc3-c3ccccc3)cc21]

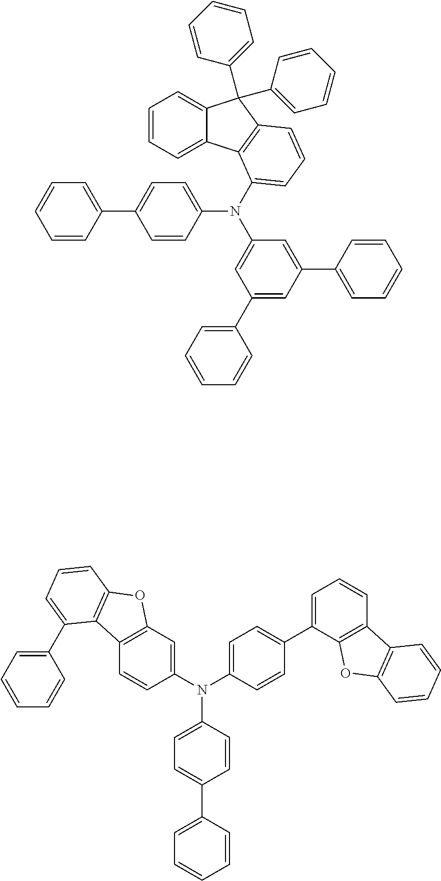 [SMILES: c1ccc(-c2ccc(N(c3cc(-c4ccccc4)cc(-c4ccccc4)c3)c3cccc4c3-c3ccccc3C4(c3ccccc3)c3ccccc3)cc2)cc1 ; c1ccc(-c2ccc(N(c3ccc(-c4cccc5c4oc4ccccc45)cc3)c3ccc4c(c3)oc3cccc(-c5ccccc5)c34)cc2)cc1]

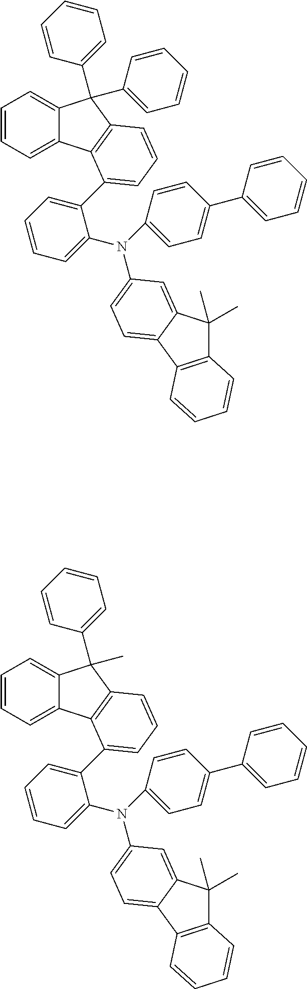 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; CC1(C)c2ccccc2-c2ccc(N(c3ccc(-c4ccccc4)cc3)c3ccccc3-c3cccc4c3-c3ccccc3C4(C)c3ccccc3)cc21]

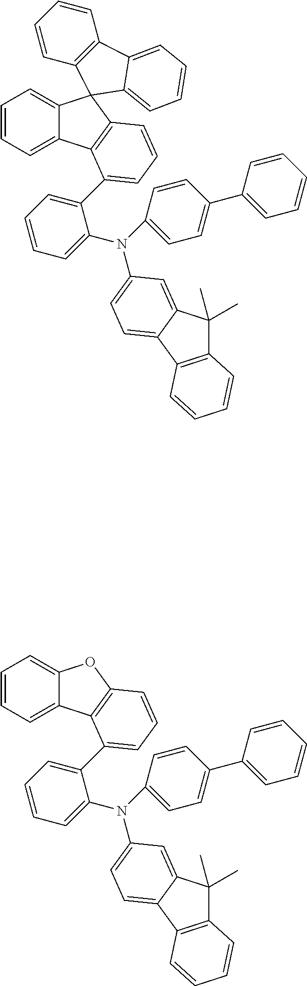 [SMILES: CC1(C)c2ccccc2-c2ccc(N(c3ccc(-c4ccccc4)cc3)c3ccccc3-c3cccc4c3-c3ccccc3C43c4ccccc4-c4ccccc43)cc21 ; CC1(C)c2ccccc2-c2ccc(N(c3ccc(-c4ccccc4)cc3)c3ccccc3-c3cccc4oc5ccccc5c34)cc21]

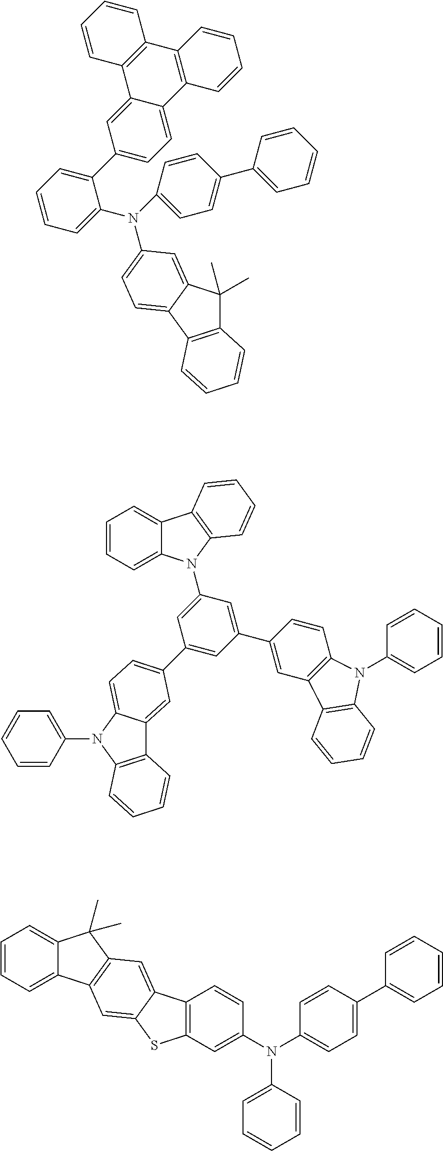 [SMILES: CC1(C)c2ccccc2-c2ccc(N(c3ccc(-c4ccccc4)cc3)c3ccccc3-c3ccc4c5ccccc5c5ccccc5c4c3)cc21 ; c1ccc(-n2c3ccccc3c3cc(-c4cc(-c5ccc6c(c5)c5ccccc5n6-c5ccccc5)cc(-n5c6ccccc6c6ccccc65)c4)ccc32)cc1 ; CC1(C)c2ccccc2-c2cc3sc4cc(N(c5ccccc5)c5ccc(-c6ccccc6)cc5)ccc4c3cc21]

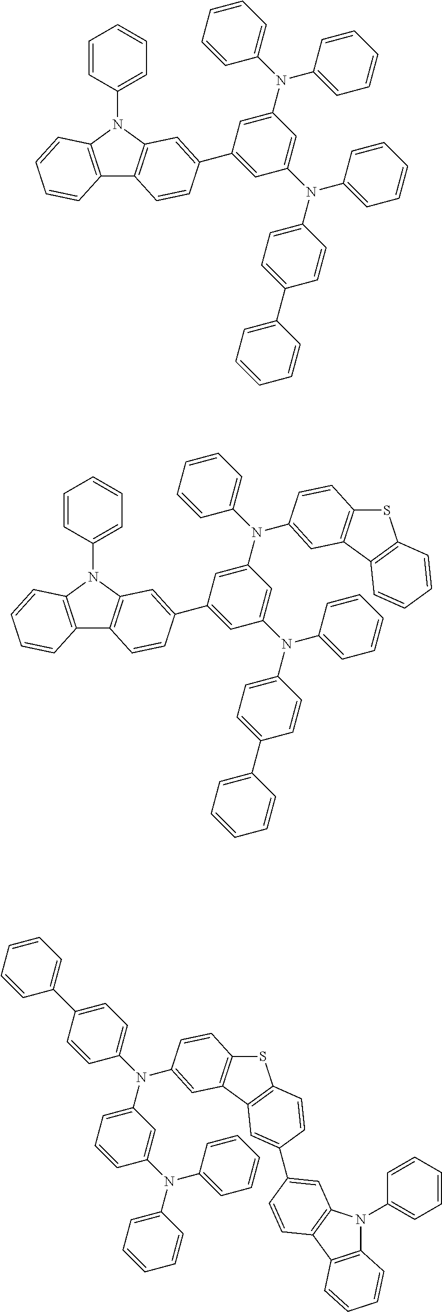 [SMILES: c1ccc(-c2ccc(N(c3ccccc3)c3cc(-c4ccc5c6ccccc6n(-c6ccccc6)c5c4)cc(N(c4ccccc4)c4ccccc4)c3)cc2)cc1 ; c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1 ; c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1]

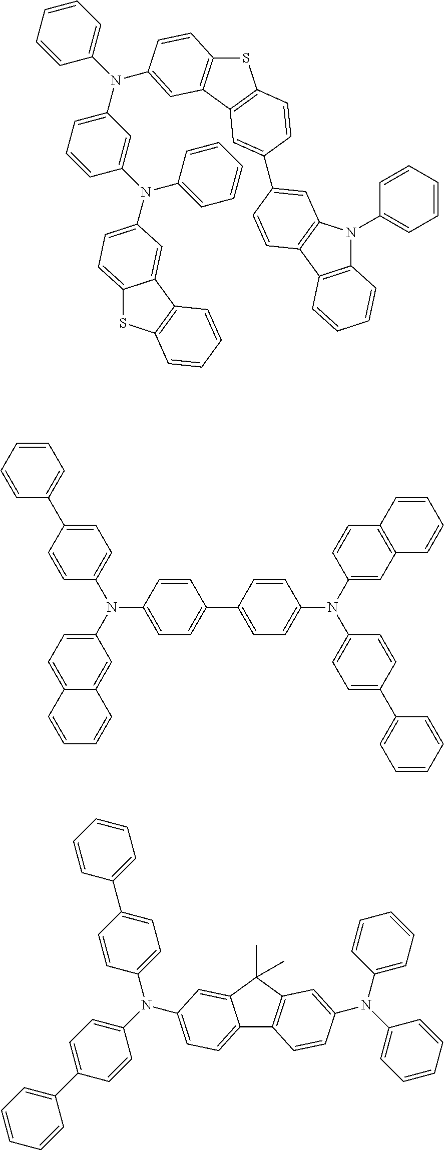 [SMILES: c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1 ; c1ccc(-c2ccc(N(c3ccc(-c4ccc(N(c5ccc(-c6ccccc6)cc5)c5ccc6ccccc6c5)cc4)cc3)c3ccc4ccccc4c3)cc2)cc1 ; CC1(C)c2cc(N(c3ccccc3)c3ccccc3)ccc2-c2ccc(N(c3ccc(-c4ccccc4)cc3)c3ccc(-c4ccccc4)cc3)cc21]

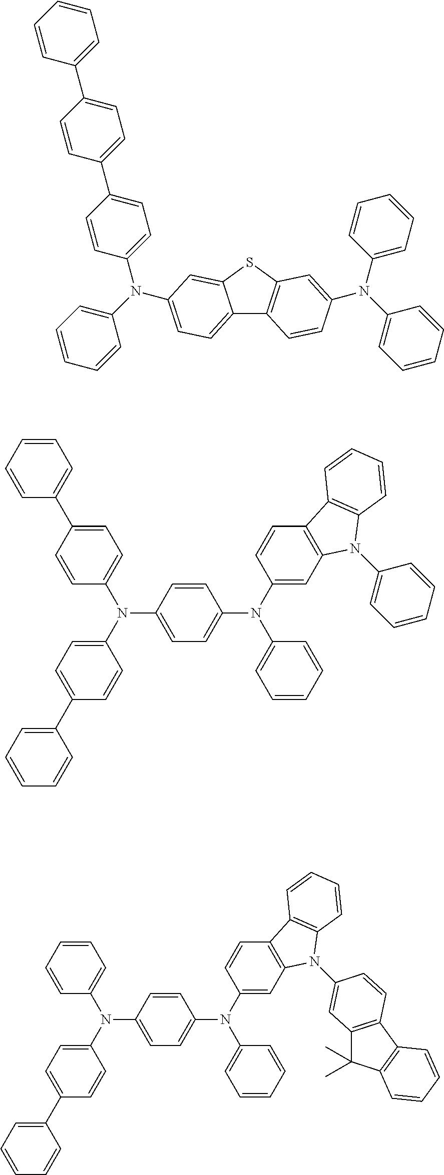 [SMILES: c1ccc(-c2ccc(-c3ccc(N(c4ccccc4)c4ccc5c(c4)sc4cc(N(c6ccccc6)c6ccccc6)ccc45)cc3)cc2)cc1 ; c1ccc(-c2ccc(N(c3ccc(-c4ccccc4)cc3)c3ccc(N(c4ccccc4)c4ccc5c6ccccc6n(-c6ccccc6)c5c4)cc3)cc2)cc1 ; CC1(C)c2ccccc2-c2ccc(-n3c4ccccc4c4ccc(N(c5ccccc5)c5ccc(N(c6ccccc6)c6ccc(-c7ccccc7)cc6)cc5)cc43)cc21]

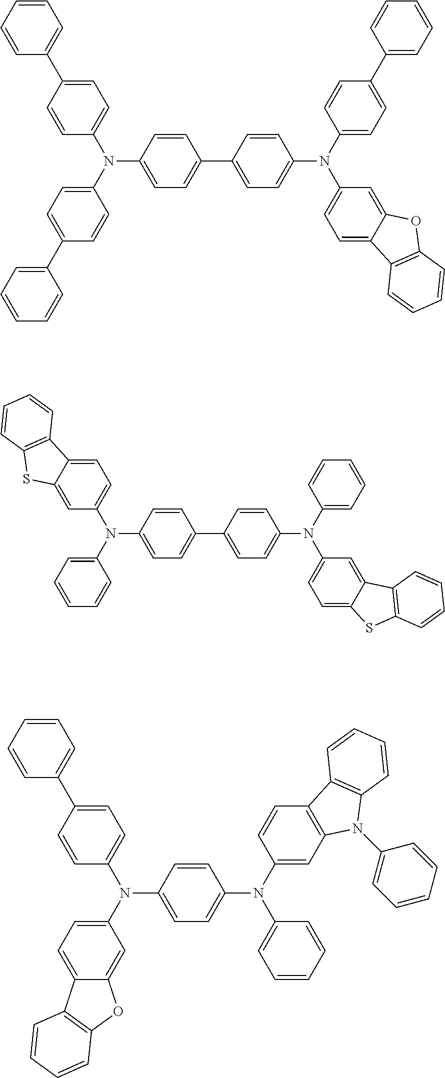 [SMILES: c1ccc(-c2ccc(N(c3ccc(-c4ccccc4)cc3)c3ccc(-c4ccc(N(c5ccc(-c6ccccc6)cc5)c5ccc6c(c5)oc5ccccc56)cc4)cc3)cc2)cc1 ; c1ccc(N(c2ccc(-c3ccc(N(c4ccccc4)c4ccc5sc6ccccc6c5c4)cc3)cc2)c2ccc3c(c2)sc2ccccc23)cc1 ; c1ccc(-c2ccc(N(c3ccc(N(c4ccccc4)c4ccc5c6ccccc6n(-c6ccccc6)c5c4)cc3)c3ccc4c(c3)oc3ccccc34)cc2)cc1]

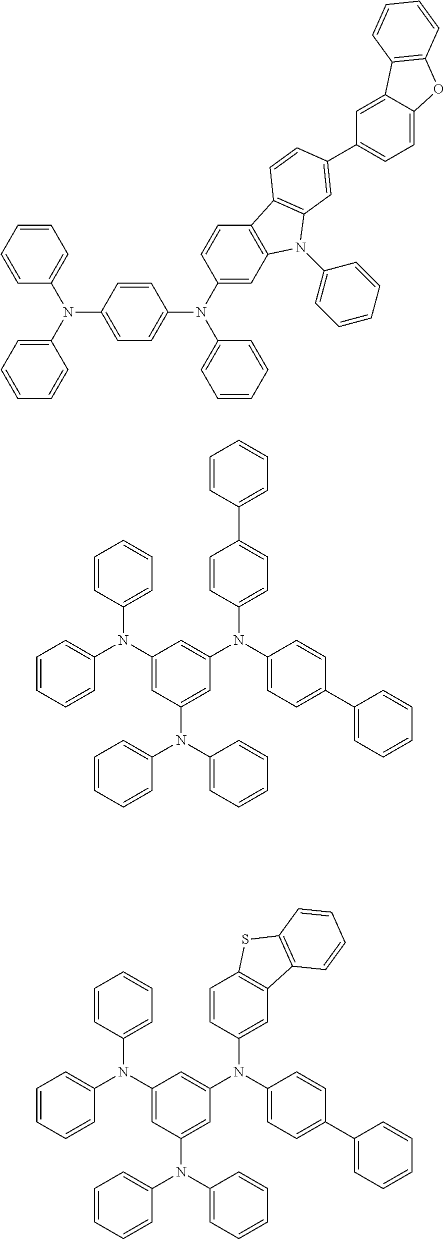

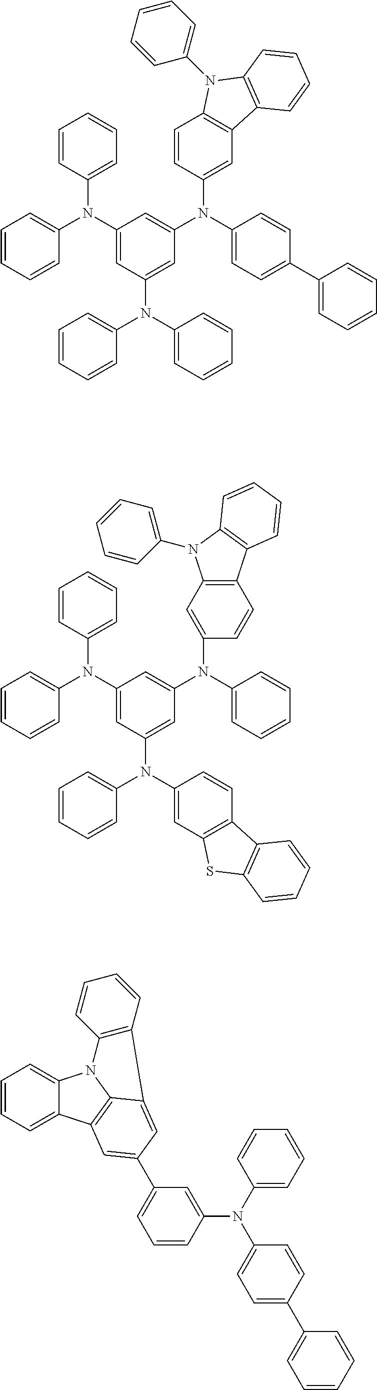 [SMILES: c1ccc(-c2ccc(N(c3cc(N(c4ccccc4)c4ccccc4)cc(N(c4ccccc4)c4ccccc4)c3)c3ccc4c(c3)c3ccccc3n4-c3ccccc3)cc2)cc1 ; c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1 ; c1ccc(-c2ccc(N(c3ccccc3)c3cccc(-c4cc5c6ccccc6n6c7ccccc7c(c4)c56)c3)cc2)cc1]

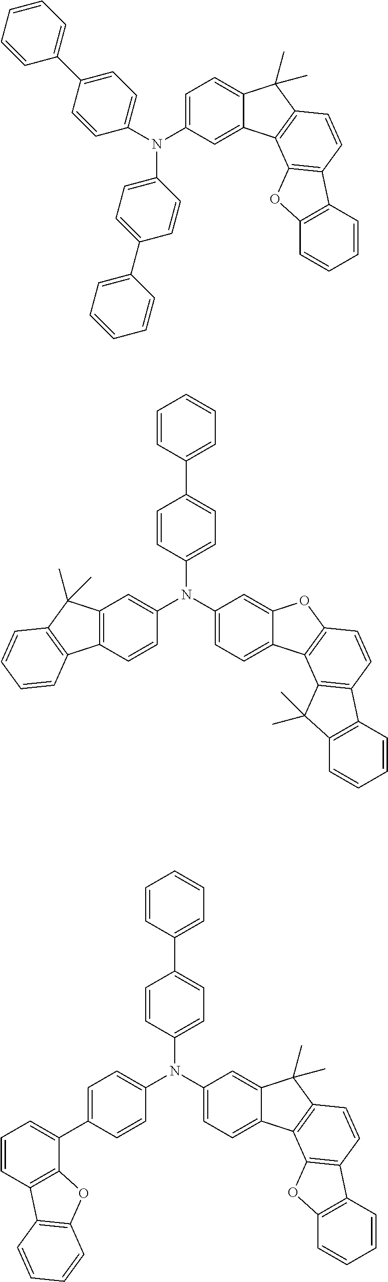 [SMILES: CC1(C)c2ccc(N(c3ccc(-c4ccccc4)cc3)c3ccc(-c4ccccc4)cc3)cc2-c2c1ccc1c2oc2ccccc21 ; CC1(C)c2ccccc2-c2ccc(N(c3ccc(-c4ccccc4)cc3)c3ccc4c(c3)oc3ccc5c(c34)C(C)(C)c3ccccc3-5)cc21 ; CC1(C)c2cc(N(c3ccc(-c4ccccc4)cc3)c3ccc(-c4cccc5c4oc4ccccc45)cc3)ccc2-c2c1ccc1c2oc2ccccc21]

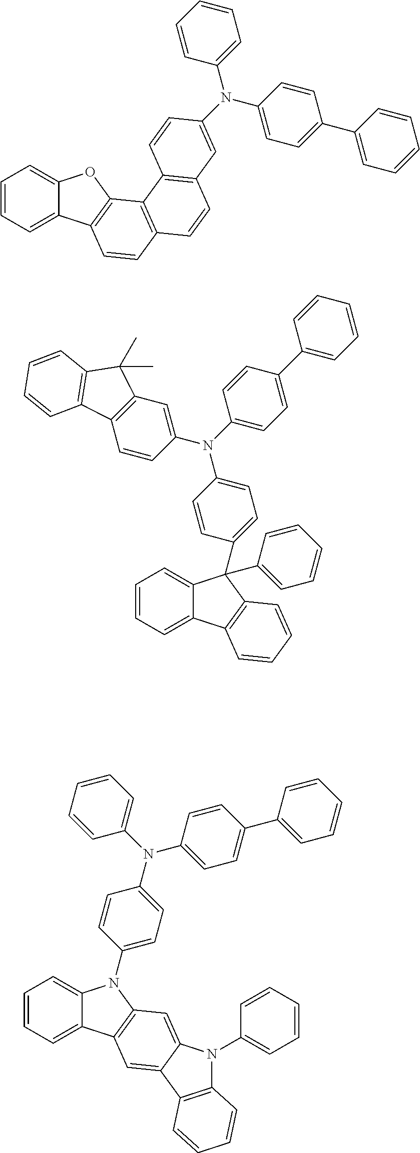 [SMILES: c1ccc(-c2ccc(N(c3ccccc3)c3ccc4c(ccc5ccc6c7ccccc7oc6c54)c3)cc2)cc1 ; CC1(C)c2ccccc2-c2ccc(N(c3ccc(-c4ccccc4)cc3)c3ccc(C4(c5ccccc5)c5ccccc5-c5ccccc54)cc3)cc21 ; c1ccc(-c2ccc(N(c3ccccc3)c3ccc(-n4c5ccccc5c5cc6c7ccccc7n(-c7ccccc7)c6cc54)cc3)cc2)cc1]

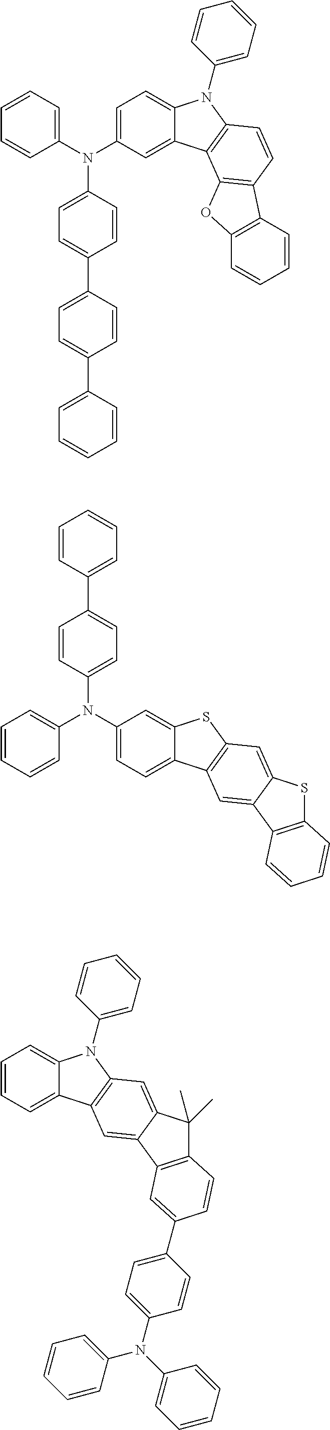 [SMILES: c1ccc(-c2ccc(-c3ccc(N(c4ccccc4)c4ccc5c(c4)c4c6oc7ccccc7c6ccc4n5-c4ccccc4)cc3)cc2)cc1 ; c1ccc(-c2ccc(N(c3ccccc3)c3ccc4c(c3)sc3cc5sc6ccccc6c5cc34)cc2)cc1 ; CC1(C)c2ccc(-c3ccc(N(c4ccccc4)c4ccccc4)cc3)cc2-c2cc3c4ccccc4n(-c4ccccc4)c3cc21]

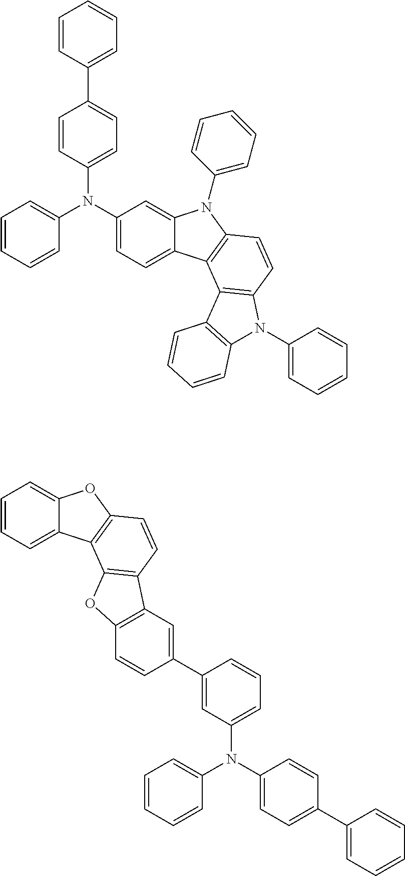 [SMILES: c1ccc(-c2ccc(N(c3ccccc3)c3ccc4c5c6c7ccccc7n(-c7ccccc7)c6ccc5n(-c5ccccc5)c4c3)cc2)cc1 ; c1ccc(-c2ccc(N(c3ccccc3)c3cccc(-c4ccc5oc6c(ccc7oc8ccccc8c76)c5c4)c3)cc2)cc1]

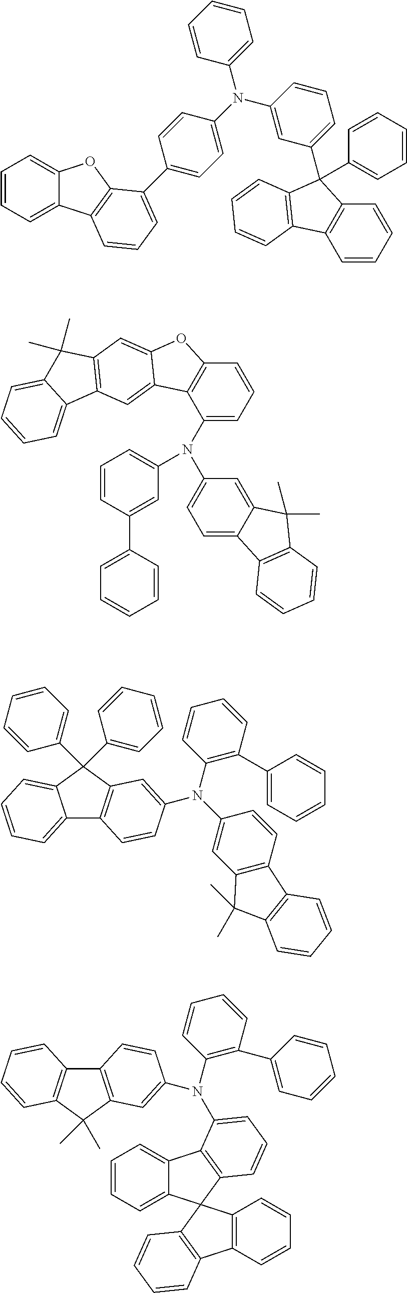 [SMILES: c1ccc(N(c2ccc(-c3cccc4c3oc3ccccc34)cc2)c2cccc(C3(c4ccccc4)c4ccccc4-c4ccccc43)c2)cc1 ; CC1(C)c2ccccc2-c2ccc(N(c3cccc(-c4ccccc4)c3)c3cccc4oc5cc6c(cc5c34)-c3ccccc3C6(C)C)cc21 ; CC1(C)c2ccccc2-c2ccc(N(c3ccc4c(c3)C(c3ccccc3)(c3ccccc3)c3ccccc3-4)c3ccccc3-c3ccccc3)cc21 ; CC1(C)c2ccccc2-c2ccc(N(c3ccccc3-c3ccccc3)c3cccc4c3-c3ccccc3C43c4ccccc4-c4ccccc43)cc21]

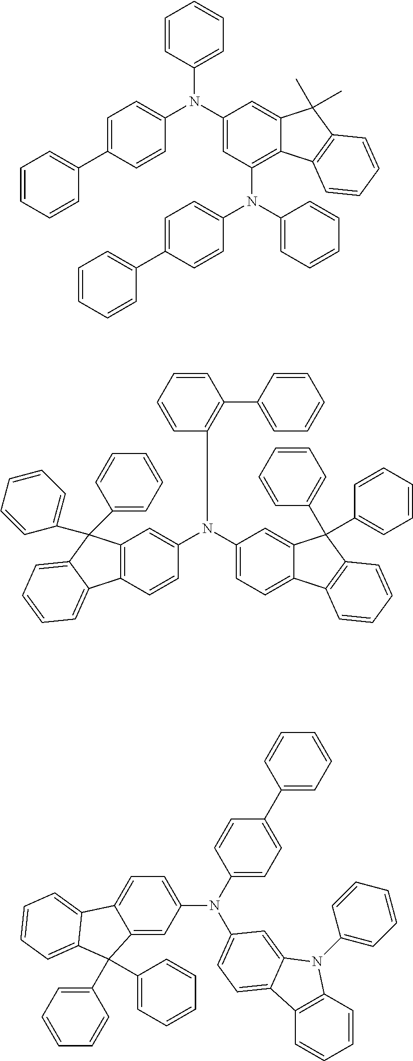 [SMILES: CC1(C)c2ccccc2-c2c(N(c3ccccc3)c3ccc(-c4ccccc4)cc3)cc(N(c3ccccc3)c3ccc(-c4ccccc4)cc3)cc21 ; c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1]

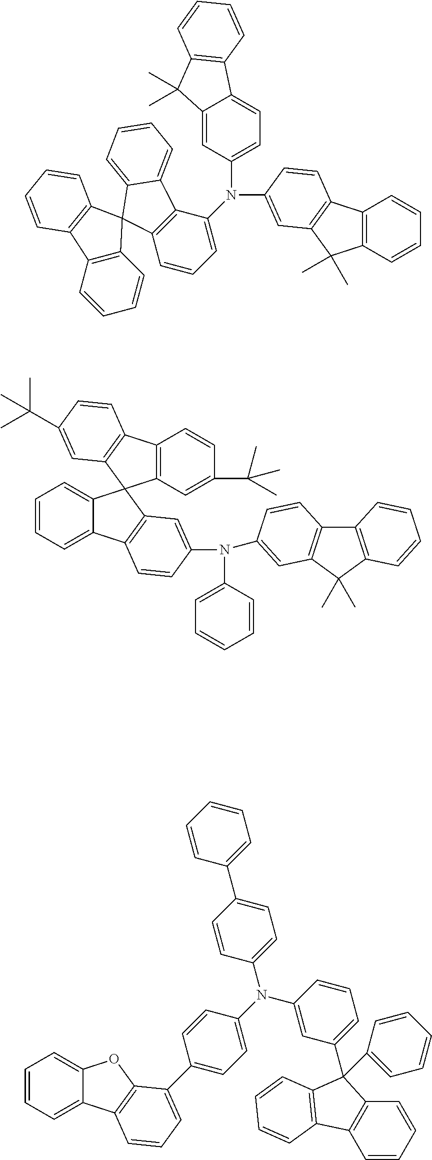 [SMILES: CC1(C)c2ccccc2-c2ccc(N(c3ccc4c(c3)C(C)(C)c3ccccc3-4)c3cccc4c3-c3ccccc3C43c4ccccc4-c4ccccc43)cc21 ; CC(C)(C)c1ccc2c(c1)C1(c3ccccc3-c3ccc(N(c4ccccc4)c4ccc5c(c4)C(C)(C)c4ccccc4-5)cc31)c1cc(C(C)(C)C)ccc1-2 ; c1ccc(-c2ccc(N(c3ccc(-c4cccc5c4oc4ccccc45)cc3)c3cccc(C4(c5ccccc5)c5ccccc5-c5ccccc54)c3)cc2)cc1]

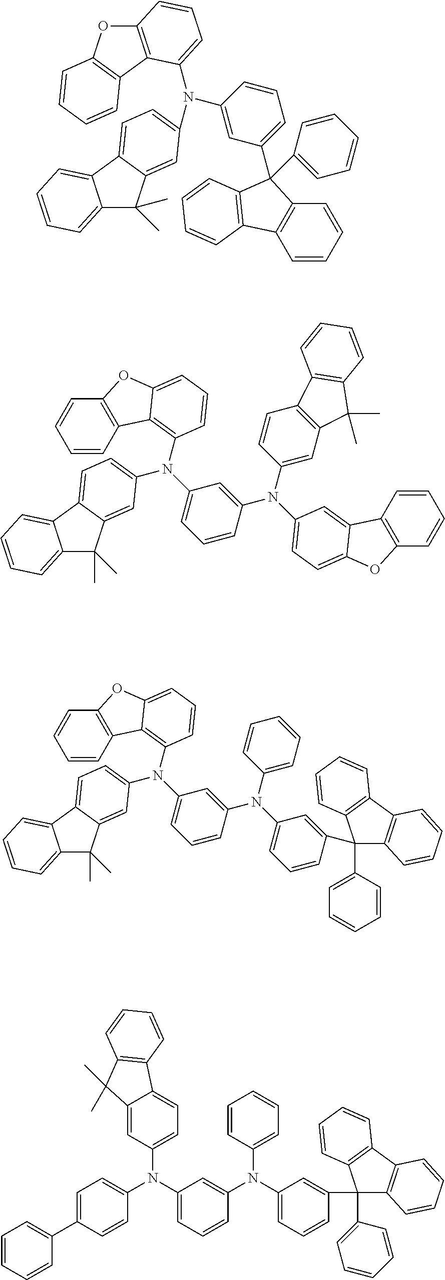 [SMILES: CC1(C)c2ccccc2-c2ccc(N(c3cccc(C4(c5ccccc5)c5ccccc5-c5ccccc54)c3)c3cccc4oc5ccccc5c34)cc21 ; CC1(C)c2ccccc2-c2ccc(N(c3cccc(N(c4ccc5c(c4)C(C)(C)c4ccccc4-5)c4cccc5oc6ccccc6c45)c3)c3ccc4oc5ccccc5c4c3)cc21 ; CC1(C)c2ccccc2-c2ccc(N(c3cccc(N(c4ccccc4)c4cccc(C5(c6ccccc6)c6ccccc6-c6ccccc65)c4)c3)c3cccc4oc5ccccc5c34)cc21 ; CC1(C)c2ccccc2-c2ccc(N(c3ccc(-c4ccccc4)cc3)c3cccc(N(c4ccccc4)c4cccc(C5(c6ccccc6)c6ccccc6-c6ccccc65)c4)c3)cc21]

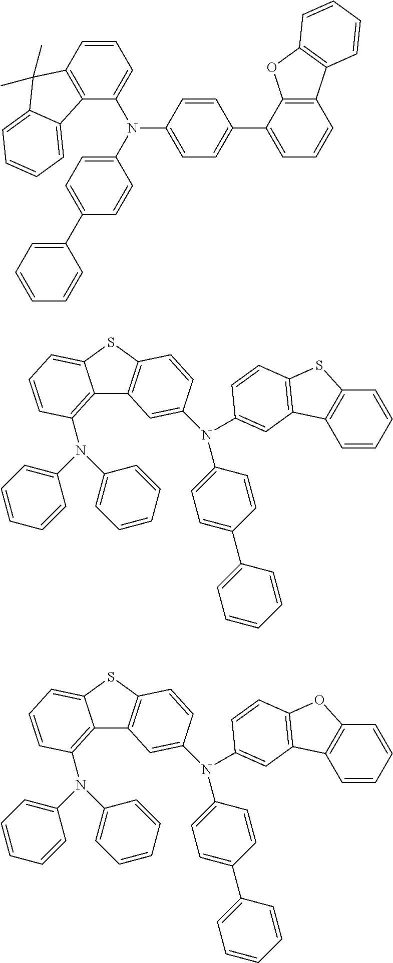 [SMILES: CC1(C)c2ccccc2-c2c(N(c3ccc(-c4ccccc4)cc3)c3ccc(-c4cccc5c4oc4ccccc45)cc3)cccc21 ; c1ccc(-c2ccc(N(c3ccc4sc5ccccc5c4c3)c3ccc4sc5cccc(N(c6ccccc6)c6ccccc6)c5c4c3)cc2)cc1 ; c1ccc(-c2ccc(N(c3ccc4oc5ccccc5c4c3)c3ccc4sc5cccc(N(c6ccccc6)c6ccccc6)c5c4c3)cc2)cc1]

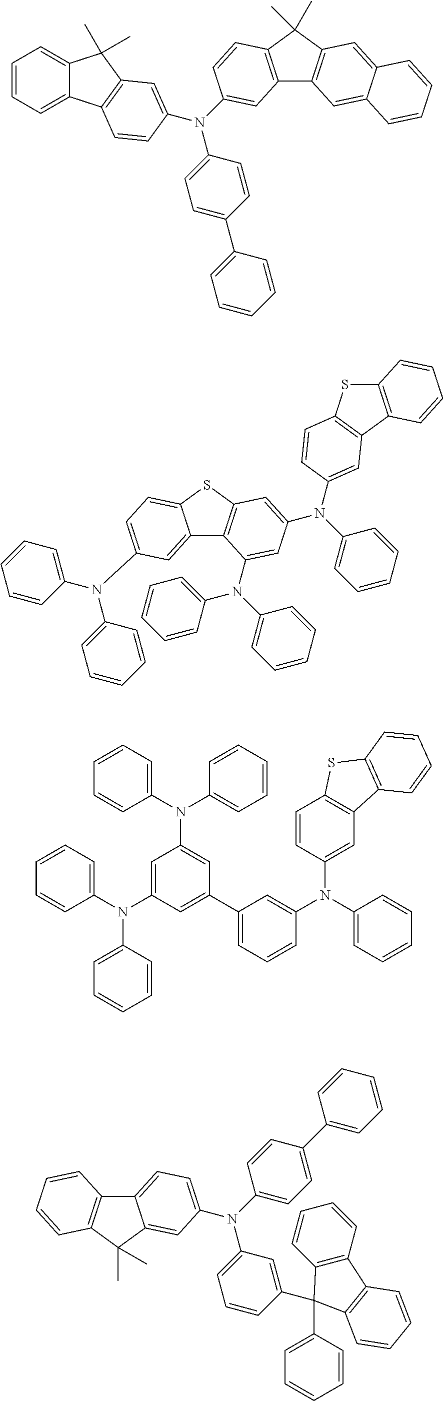 [SMILES: CC1(C)c2ccccc2-c2ccc(N(c3ccc(-c4ccccc4)cc3)c3ccc4c(c3)-c3cc5ccccc5cc3C4(C)C)cc21 ; c1ccc(N(c2cc(N(c3ccccc3)c3ccccc3)c3c(c2)sc2ccc(N(c4ccccc4)c4ccccc4)cc23)c2ccc3sc4ccccc4c3c2)cc1 ; c1ccc(N(c2ccccc2)c2cc(-c3cccc(N(c4ccccc4)c4ccc5sc6ccccc6c5c4)c3)cc(N(c3ccccc3)c3ccccc3)c2)cc1 ; CC1(C)c2ccccc2-c2ccc(N(c3ccc(-c4ccccc4)cc3)c3cccc(C4(c5ccccc5)c5ccccc5-c5ccccc54)c3)cc21]

In the hole transport region 140, other suitable compounds may be used in addition to the compounds.

In an implementation, the charge transport region may be, e.g., the electron transport region 150.

The electron transport region 150 may help further increase electron injection and/or electron mobility between the cathode 110 and the light emitting layer 130 and block holes.

In an implementation, the electron transport region 150 may include an electron transport layer between the cathode 110 and the light emitting layer 130, and an electron transport auxiliary layer between the light emitting layer 130 and the electron transport layer and at least one of the compounds of Group C may be included in at least one of the electron transport layer and the electron transport auxiliary layer.

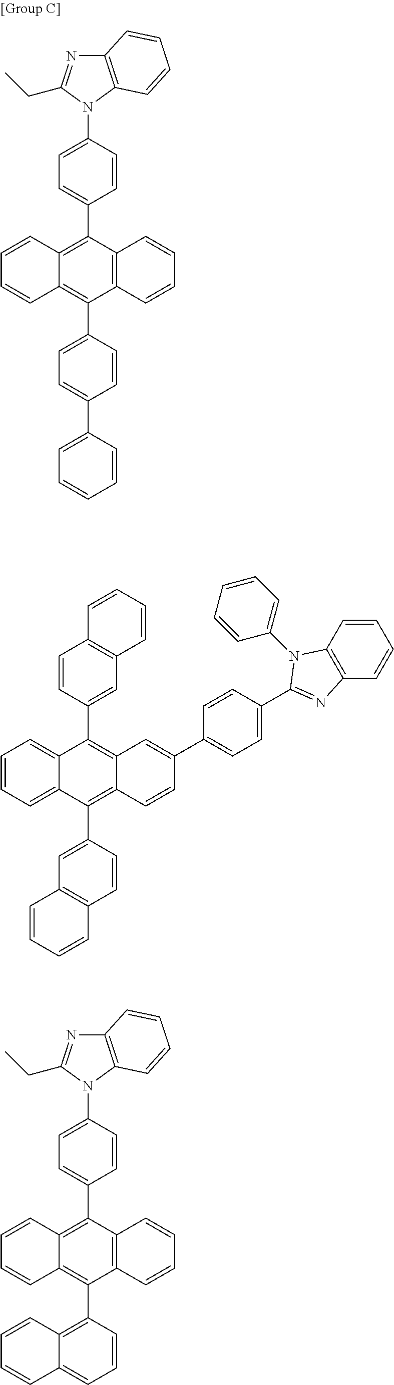 [SMILES: CCc1nc2ccccc2n1-c1ccc(-c2c3ccccc3c(-c3ccc(-c4ccccc4)cc3)c3ccccc23)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; CCc1nc2ccccc2n1-c1ccc(-c2c3ccccc3c(-c3cccc4ccccc34)c3ccccc23)cc1]

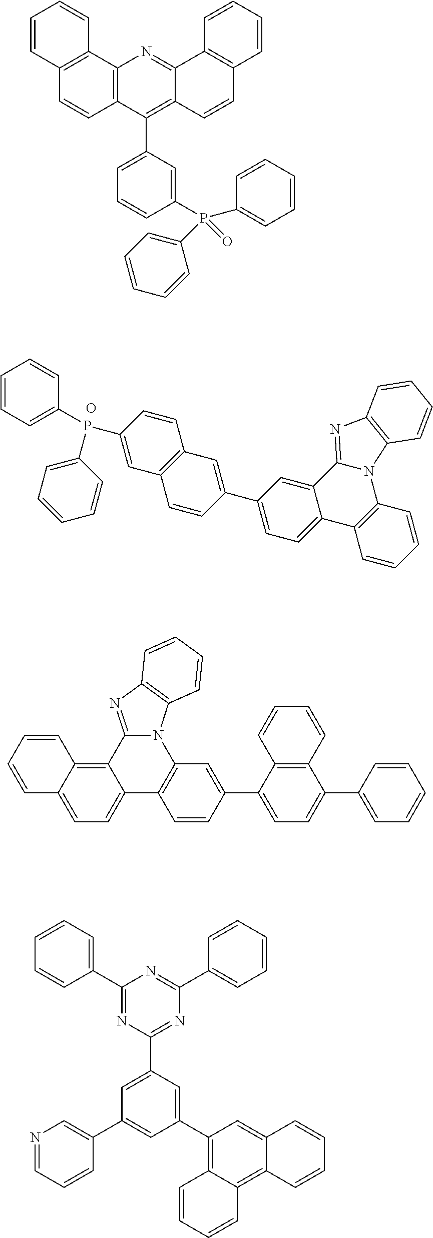

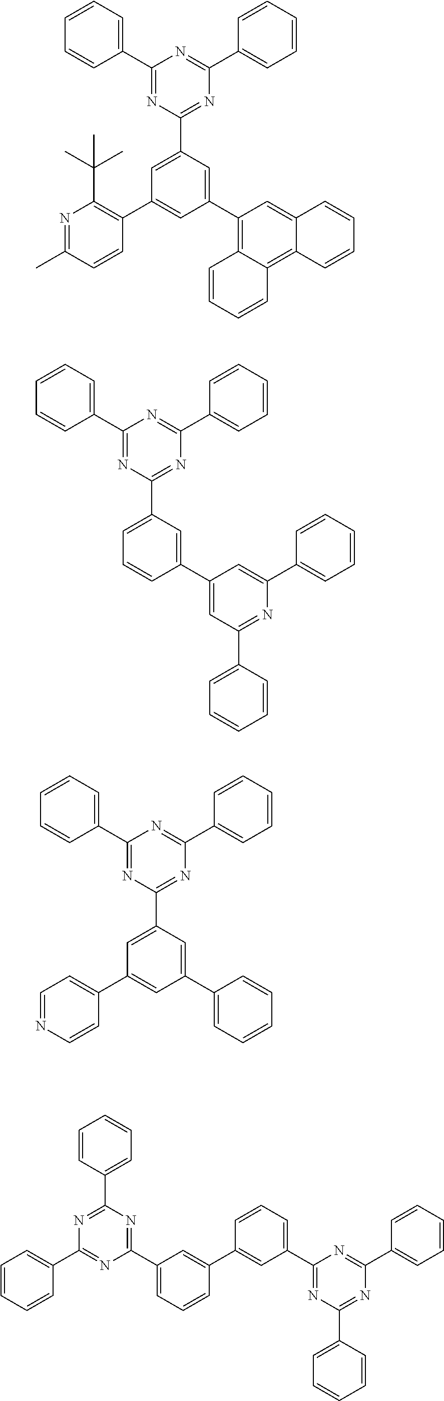

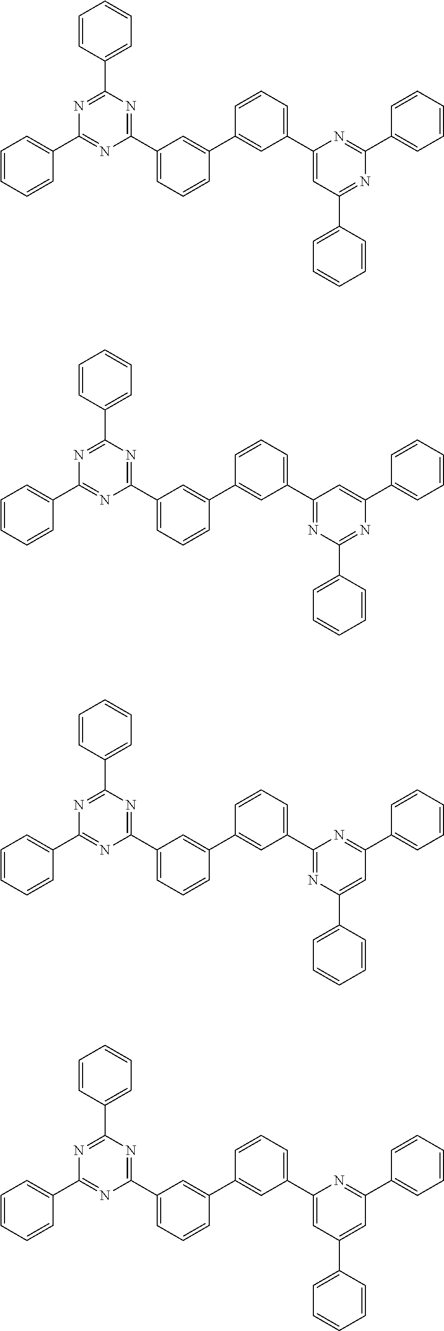 [SMILES: c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1]

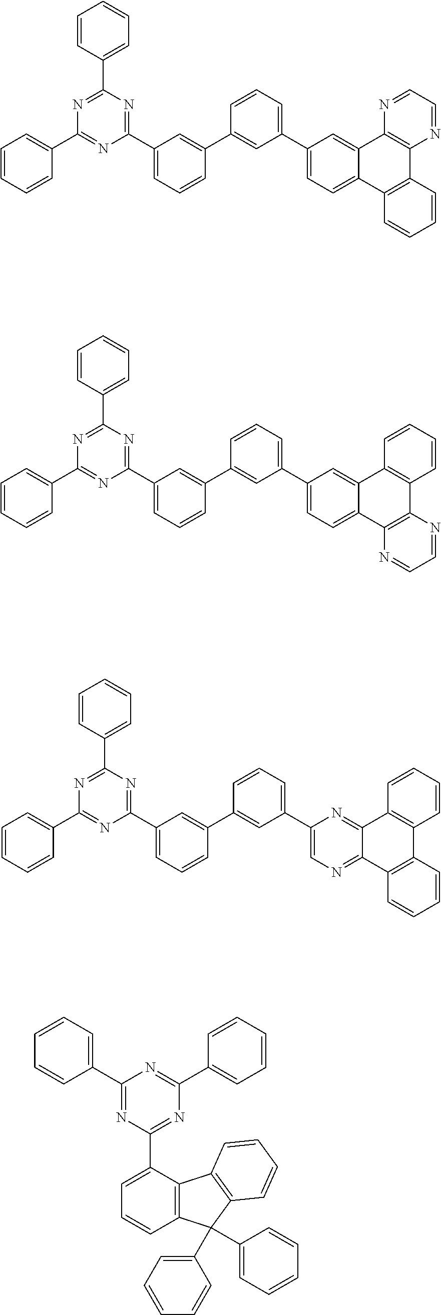 [SMILES: c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1 ; c1ccc(-c2nc(-c3ccccc3)nc(-c3cccc(-c4cccc(-c5cnc6c7ccccc7c7ccccc7c6n5)c4)c3)n2)cc1 ; c1ccc(-c2nc(-c3ccccc3)nc(-c3cccc4c3-c3ccccc3C4(c3ccccc3)c3ccccc3)n2)cc1]

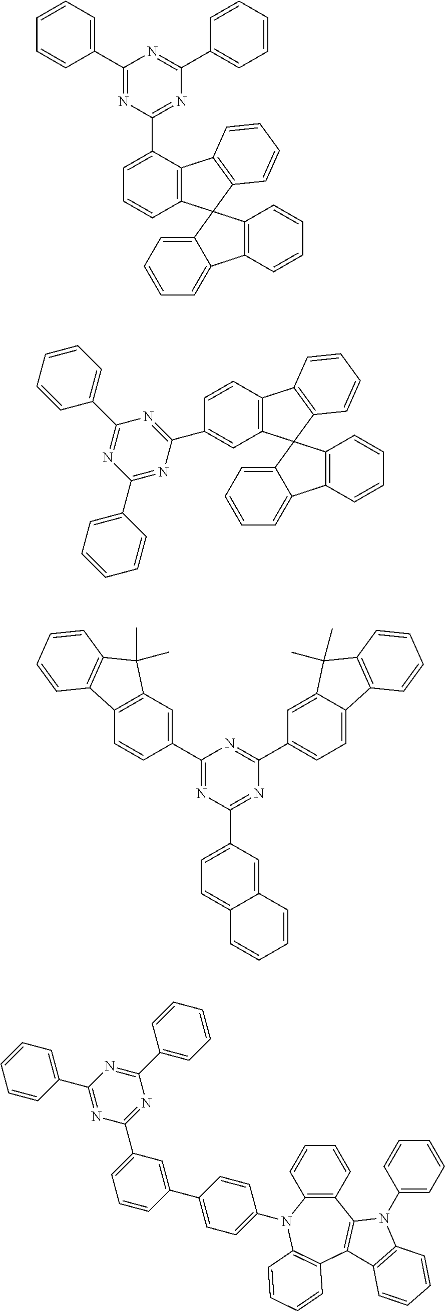 [SMILES: c1ccc(-c2nc(-c3ccccc3)nc(-c3cccc4c3-c3ccccc3C43c4ccccc4-c4ccccc43)n2)cc1 ; c1ccc(-c2nc(-c3ccccc3)nc(-c3ccc4c(c3)C3(c5ccccc5-c5ccccc53)c3ccccc3-4)n2)cc1 ; CC1(C)c2ccccc2-c2ccc(-c3nc(-c4ccc5c(c4)C(C)(C)c4ccccc4-5)nc(-c4ccc5ccccc5c4)n3)cc21 ; c1ccc(-c2nc(-c3ccccc3)nc(-c3cccc(-c4ccc(N5c6ccccc6-c6c(n(-c7ccccc7)c7ccccc67)-c6ccccc65)cc4)c3)n2)cc1]

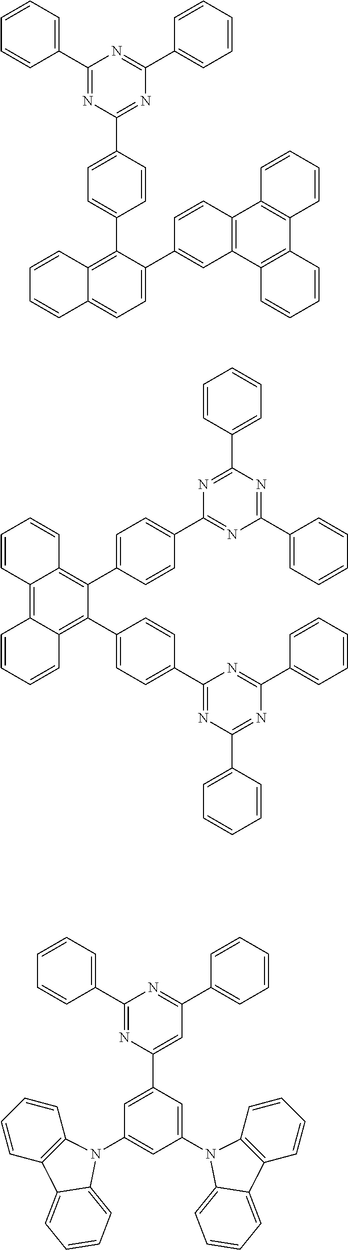 [SMILES: c1ccc(-c2nc(-c3ccccc3)nc(-c3ccc(-c4c(-c5ccc6c7ccccc7c7ccccc7c6c5)ccc5ccccc45)cc3)n2)cc1 ; c1ccc(-c2nc(-c3ccccc3)nc(-c3ccc(-c4c(-c5ccc(-c6nc(-c7ccccc7)nc(-c7ccccc7)n6)cc5)c5ccccc5c5ccccc45)cc3)n2)cc1 ; c1ccc(-c2cc(-c3cc(-n4c5ccccc5c5ccccc54)cc(-n4c5ccccc5c5ccccc54)c3)nc(-c3ccccc3)n2)cc1]

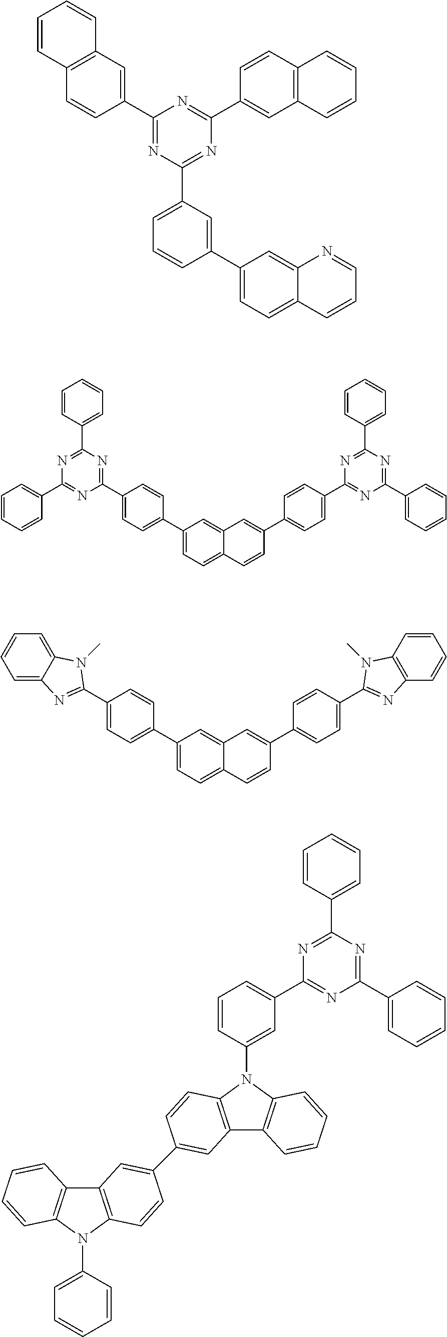 [SMILES: c1cc(-c2ccc3cccnc3c2)cc(-c2nc(-c3ccc4ccccc4c3)nc(-c3ccc4ccccc4c3)n2)c1 ; c1ccc(-c2nc(-c3ccccc3)nc(-c3ccc(-c4ccc5ccc(-c6ccc(-c7nc(-c8ccccc8)nc(-c8ccccc8)n7)cc6)cc5c4)cc3)n2)cc1 ; Cn1c(-c2ccc(-c3ccc4ccc(-c5ccc(-c6nc7ccccc7n6C)cc5)cc4c3)cc2)nc2ccccc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1]

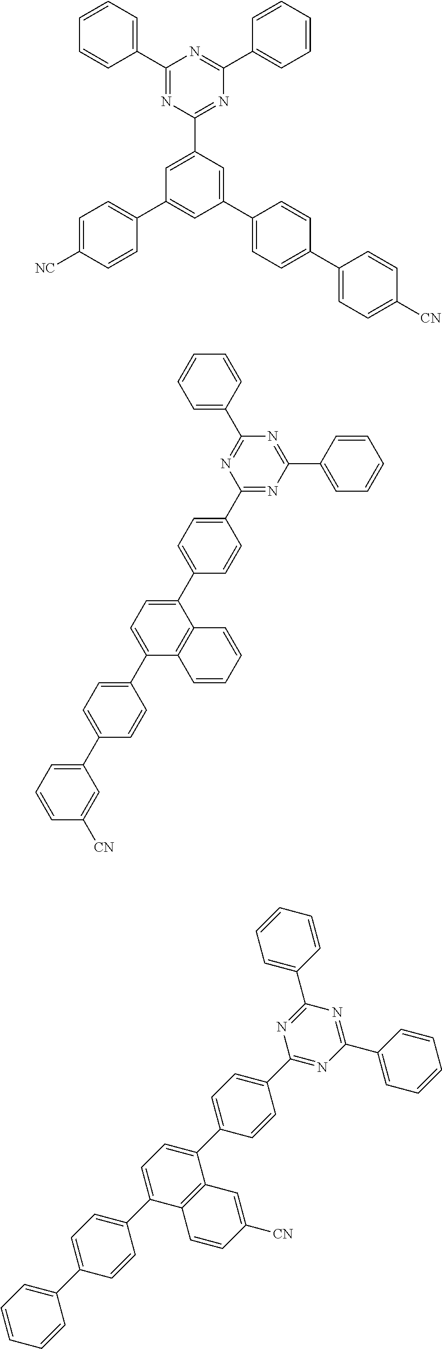 [SMILES: N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1]

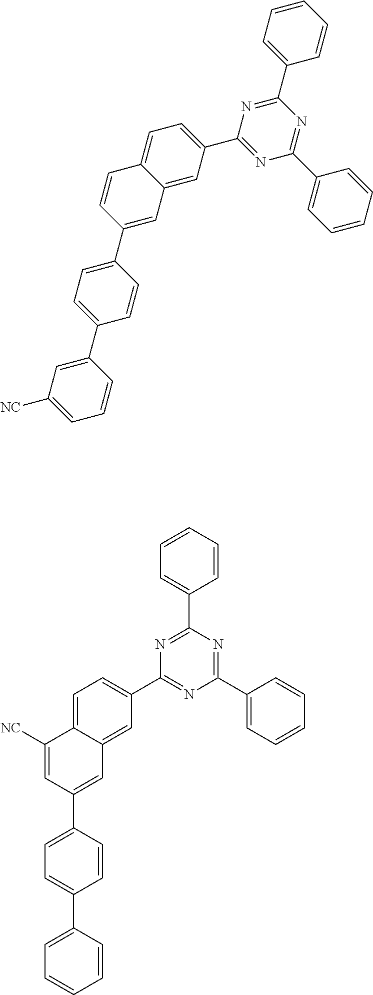 [SMILES: N#Cc1cccc(-c2ccc(-c3ccc4ccc(-c5nc(-c6ccccc6)nc(-c6ccccc6)n5)cc4c3)cc2)c1 ; N#Cc1cc(-c2ccc(-c3ccccc3)cc2)cc2cc(-c3nc(-c4ccccc4)nc(-c4ccccc4)n3)ccc12]

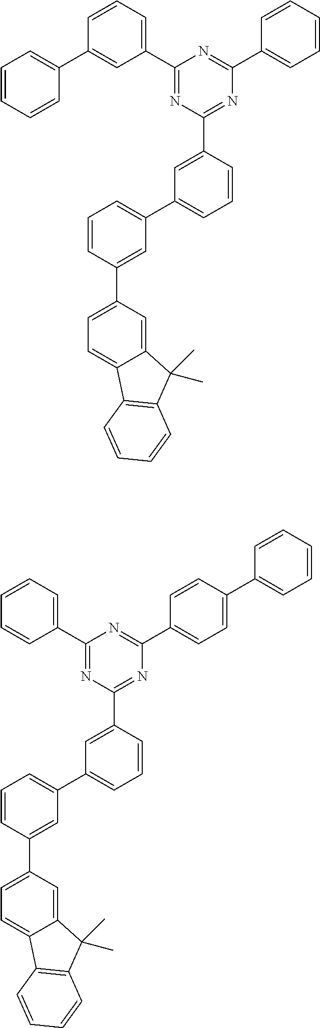 [SMILES: CC1(C)c2ccccc2-c2ccc(-c3cccc(-c4cccc(-c5nc(-c6ccccc6)nc(-c6cccc(-c7ccccc7)c6)n5)c4)c3)cc21 ; CC1(C)c2ccccc2-c2ccc(-c3cccc(-c4cccc(-c5nc(-c6ccccc6)nc(-c6ccc(-c7ccccc7)cc6)n5)c4)c3)cc21]

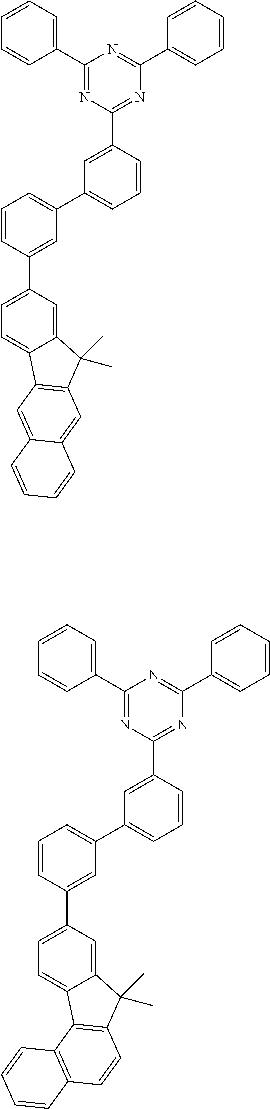 [SMILES: CC1(C)c2cc(-c3cccc(-c4cccc(-c5nc(-c6ccccc6)nc(-c6ccccc6)n5)c4)c3)ccc2-c2cc3ccccc3cc21 ; CC1(C)c2cc(-c3cccc(-c4cccc(-c5nc(-c6ccccc6)nc(-c6ccccc6)n5)c4)c3)ccc2-c2c1ccc1ccccc21]

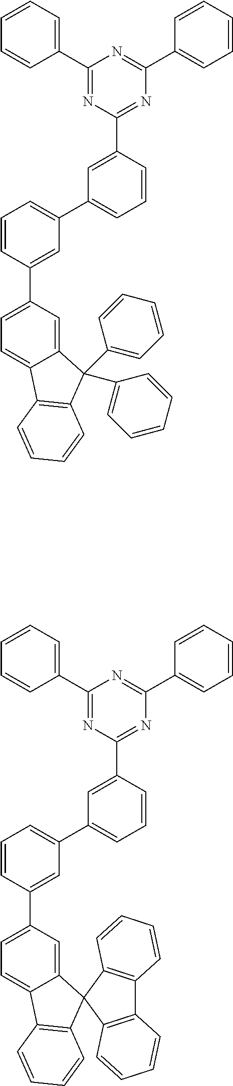 [SMILES: c1ccc(-c2nc(-c3ccccc3)nc(-c3cccc(-c4cccc(-c5ccc6c(c5)C(c5ccccc5)(c5ccccc5)c5ccccc5-6)c4)c3)n2)cc1 ; c1ccc(-c2nc(-c3ccccc3)nc(-c3cccc(-c4cccc(-c5ccc6c(c5)C5(c7ccccc7-c7ccccc75)c5ccccc5-6)c4)c3)n2)cc1]

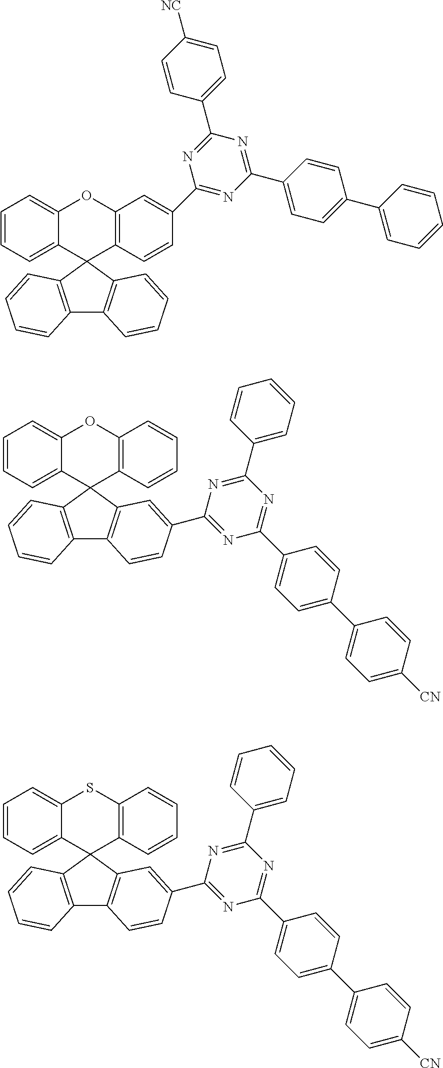 [SMILES: N#Cc1ccc(-c2nc(-c3ccc(-c4ccccc4)cc3)nc(-c3ccc4c(c3)Oc3ccccc3C43c4ccccc4-c4ccccc43)n2)cc1 ; N#Cc1ccc(-c2ccc(-c3nc(-c4ccccc4)nc(-c4ccc5c(c4)C4(c6ccccc6Oc6ccccc64)c4ccccc4-5)n3)cc2)cc1 ; N#Cc1ccc(-c2ccc(-c3nc(-c4ccccc4)nc(-c4ccc5c(c4)C4(c6ccccc6Sc6ccccc64)c4ccccc4-5)n3)cc2)cc1]

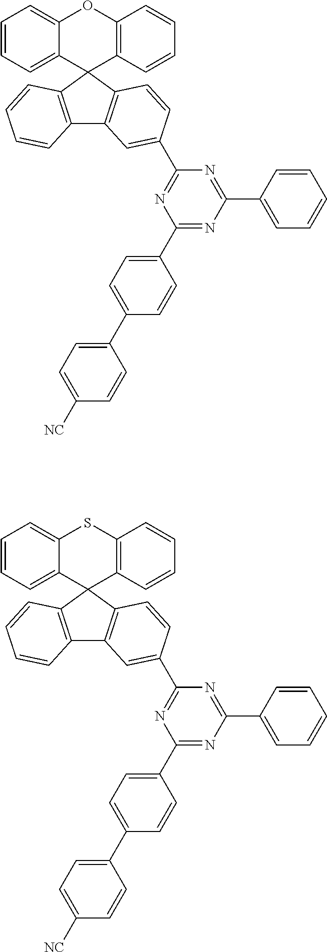 [SMILES: N#Cc1ccc(-c2ccc(-c3nc(-c4ccccc4)nc(-c4ccc5c(c4)-c4ccccc4C54c5ccccc5Oc5ccccc54)n3)cc2)cc1 ; N#Cc1ccc(-c2ccc(-c3nc(-c4ccccc4)nc(-c4ccc5c(c4)-c4ccccc4C54c5ccccc5Sc5ccccc54)n3)cc2)cc1]

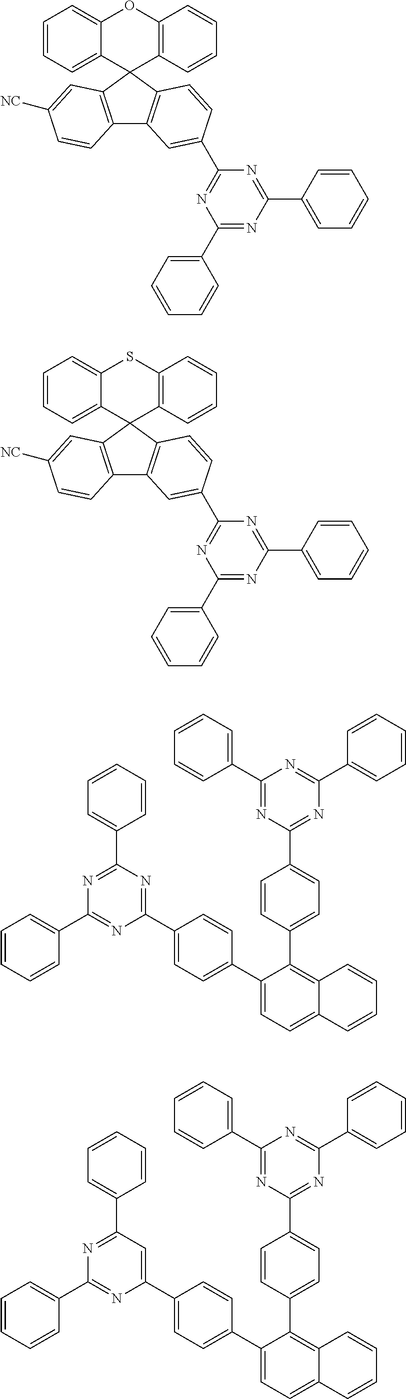

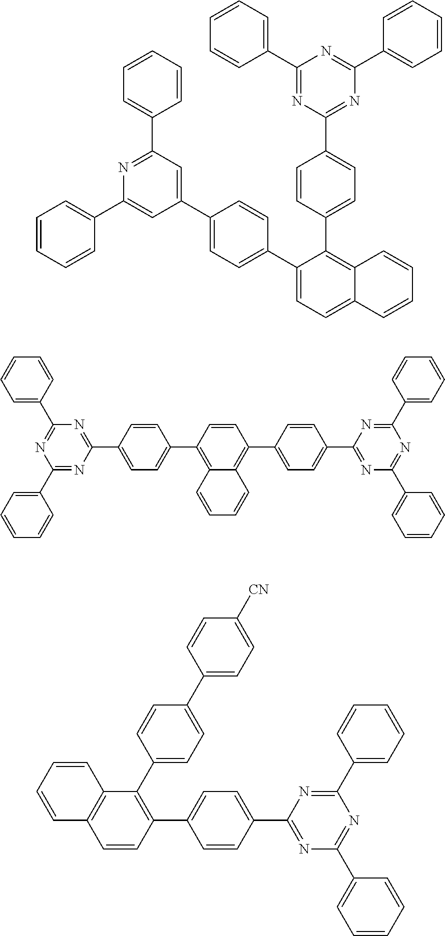

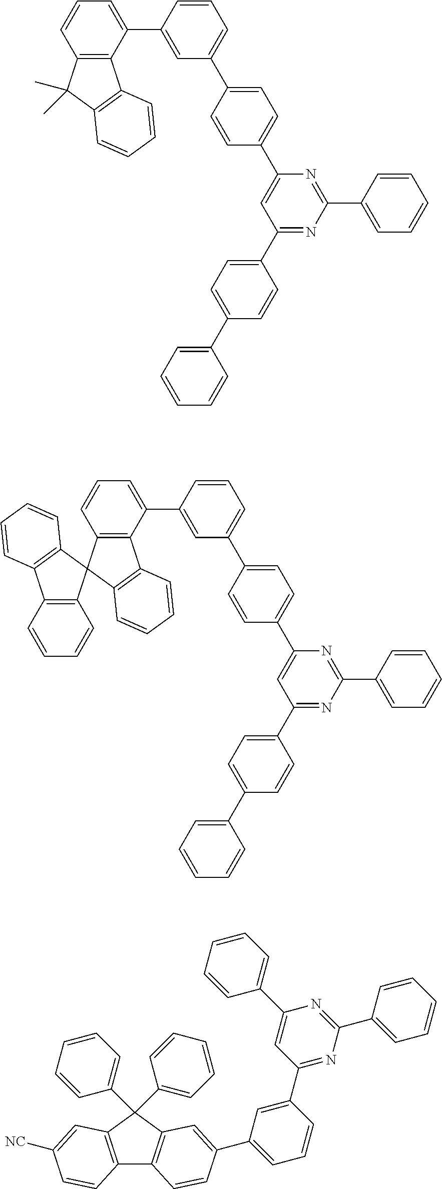 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21 ; c1ccc(-c2ccc(-c3cc(-c4ccc(-c5cccc(-c6cccc7c6-c6ccccc6C76c7ccccc7-c7ccccc76)c5)cc4)nc(-c4ccccc4)n3)cc2)cc1 ; N#Cc1ccc2c(c1)C(c1ccccc1)(c1ccccc1)c1cc(-c3cccc(-c4cc(-c5ccccc5)nc(-c5ccccc5)n4)c3)ccc1-2]

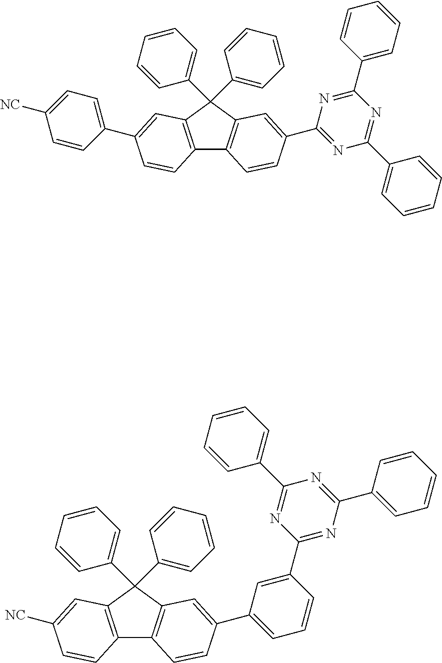 [SMILES: N#Cc1ccc(-c2ccc3c(c2)C(c2ccccc2)(c2ccccc2)c2cc(-c4nc(-c5ccccc5)nc(-c5ccccc5)n4)ccc2-3)cc1 ; N#Cc1ccc2c(c1)C(c1ccccc1)(c1ccccc1)c1cc(-c3cccc(-c4nc(-c5ccccc5)nc(-c5ccccc5)n4)c3)ccc1-2]

An embodiment may provide an organic light emitting diode including a light emitting layer as an organic layer.

Another embodiment may provide an organic light emitting diode including a light emitting layer and a hole transport region as an organic layer.

Another embodiment may provide an organic light emitting diode including a light emitting layer and an electron transport region as an organic layer.

The organic light emitting diode according to an embodiment may include a hole transport region 140 and an electron transport region 150 in addition to the light emitting layer 130 as the organic layer 105, as shown in the FIGURE.

In an implementation, the organic light emitting diode may further include an electron injection layer, a hole injection layer, or the like, in addition to the light emitting layer as the aforementioned organic layer.

The organic light emitting diode 100 may be produced by forming an anode or a cathode on a substrate, forming an organic layer using a dry film formation method such as a vacuum deposition method (evaporation), sputtering, plasma plating, or ion plating, and forming a cathode or an anode thereon.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in examples and synthesis examples were purchased from Sigma-Aldrich Co. Ltd., TCI Inc., Tokyo chemical Industry, or P&H Tech, as far as there is no particular comment, or were synthesized by suitable methods.

(Preparation of Compound for Organic Optoelectronic Device)

The compound was synthesized through the following steps.

### Preparation Synthesis Example 1: Synthesis of Compound A1

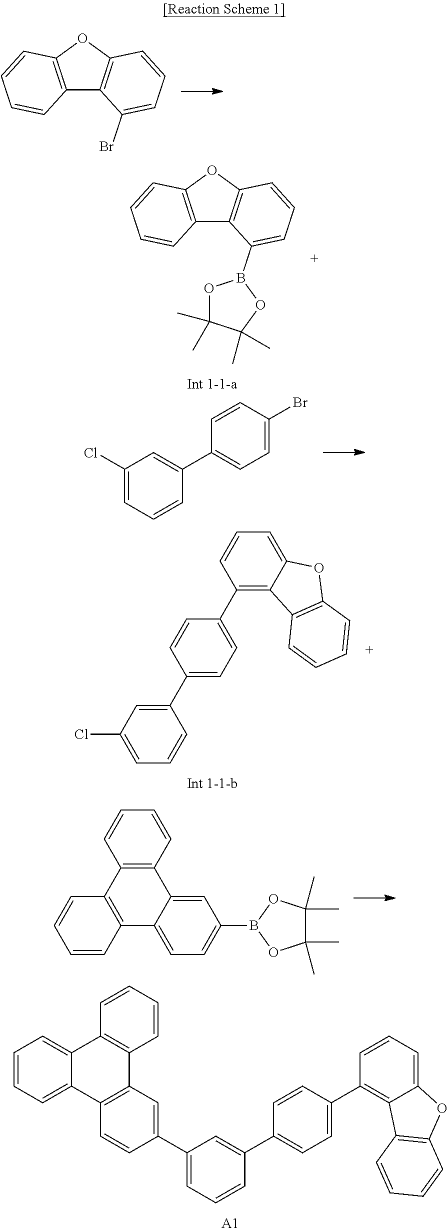

### 1st step: Synthesis of Intermediate Int 1-1-a

50 g (203.3 mmol) of 1-bromodibenzofuran, 59.4 g (233.8 mmol) of bis(pinacolato)diboron, 4.8 g (5.9 mmol) of Pd(dppf)Cl₂, and 28.9 g (294.8 mmol) of potassium acetate were put in a round-bottomed flask and dissolved in 400 ml of DMF. The mixture was stirred under reflux at 120° C. for 12 hours. When a reaction was completed, the resultant was poured into an excess of distilled water and then, stirred for 1 hour. A solid was filtered therefrom and dissolved in DCM. After removing moisture therefrom with MgSO₄, an organic solvent was filtered and removed therefrom with silica gel pad under a reduced pressure. A solid therefrom was recrystallized with ethyl acetate and hexane, obtaining 41.8 g (70%) of Intermediate Int 1-1-a.

### 2nd step: Synthesis of Intermediate Int 1-1-b

37.9 g (142.1 mmol) of 4-bromo-3′-chloro-1,1′-biphenyl, 41.8 g (142.1 mmol) of Intermediate Int 1-1-a, 29.5 g (213.2 mmol) of K₂CO₃, and 4.9 g (4.3 mmol) of Pd(PPh₃)₄ were suspended in 280 ml of THF and 110 ml of distilled water under a nitrogen flow and then, stirred under reflux for 8 hours. When a reaction was completed, the resultant was extracted with DCM and treated through column chromatography (hexane:DCM (20%)), obtaining 39.3 g (78%) of Intermediate Int 1-1-b.

### 3rd step: Synthesis of Compound A1

39.3 g (110.8 mmol) of Intermediate Int 1-1-b, 39.2 g (110.8 mmol) of 4,4,5,5-tetramethyl-2-(triphenylen-2-yl)-1,3,2-dioxaborolane, 65.0 g (199.4 mmol) of Cs₂CO₃, 5.1 g (5.5 mmol) of Pd₂(dba)₃, and 8.9 g (22.2 mmol) of P(t-Bu)₃ were suspended in 440 ml of 1,4-dioxane under a nitrogen flow and then, stirred under reflux for 8 hours. When a reaction was completed, the resultant was cooled to ambient temperature, and a solid formed by pouring an excess of methanol thereto was filtered. The solid was washed with distilled water, methanol, and acetone in order and recrystallized with 400 ml of MCB, obtaining 29.1 g (48%) of Compound A1.

LC-Mass (theoretical value: 546.66 g/mol, measured value: M+=547.51 g/mol)

### Preparation Synthesis Example 2: Synthesis of Compound A2

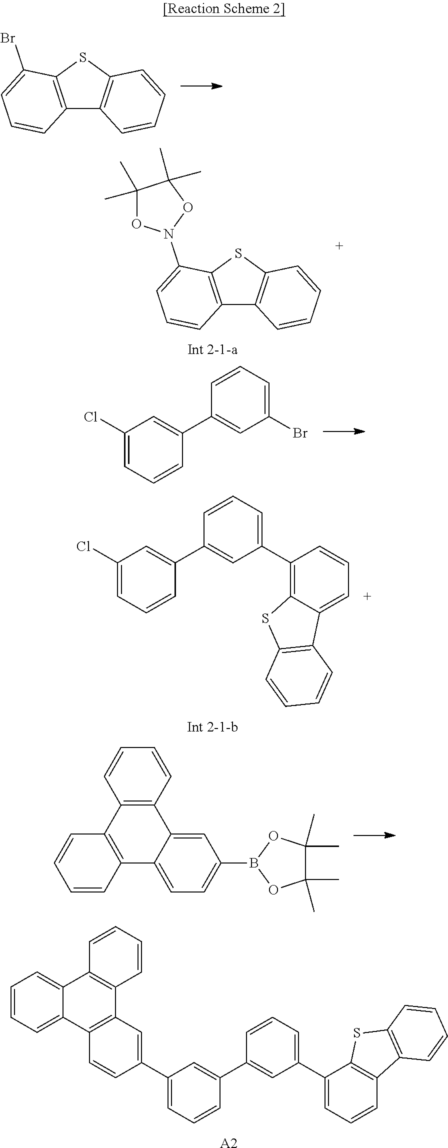

1st step: Synthesis of Intermediate Int 2-1-a

28 g (55%) of Intermediate Int 2-1-a was synthesized and purified in the same method as in the 1s^{t} step of Preparation Synthesis Example 1 except that 4-bromodibenzothiophene was used instead of the 1-bromodibenzofuran.

### 2nd step: Synthesis of Intermediate Int 2-1-b

22 g (65%) of Intermediate Int 2-1-b was synthesized and purified in the same method as in the 2nd step of Preparation Synthesis Example 1 except that 3-bromo-3′-chloro-1,1′-biphenyl was used instead of the 4-bromo-3′-chloro-1,1′-biphenyl.

### 3rd step: Synthesis of Compound A2

11.1 g (39%) of Compound A2 was synthesized and purified in the same method as in the 3rd step of Preparation Synthesis Example 1 except that Intermediate Int 2-1-b was used.

LC-Mass (theoretical value: 562.72 g/mol, measured value: M+=563.57 g/mol)

### Preparation Synthesis Example 3: Synthesis of Compound A3

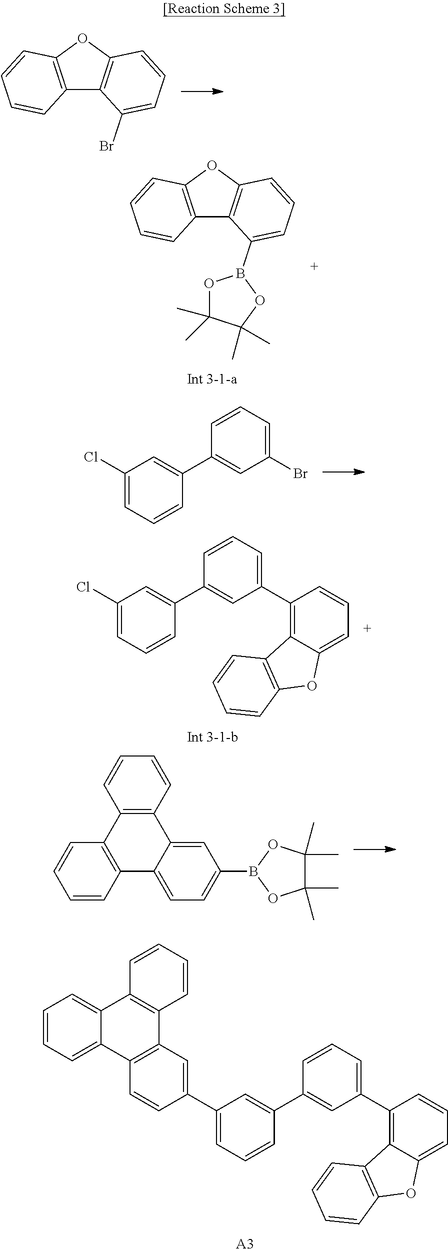

### 1st step: Synthesis of Intermediate Int 3-1-a

19.1 g (45%) of Intermediate Int 3-1-a was synthesized and purified in the same method as in the 1^{st} step of Preparation Synthesis Example 1.

### 2nd step: Synthesis of Intermediate Int 3-1-b

26.2 g (65%) of Intermediate Int 3-1-b was synthesized and purified in the same method as in the 1^{st} step of Preparation Synthesis Example 1 except that 3-bromo-3′-chloro-1,1′-biphenyl was used instead of the 4-bromo-3′-chloro-1,1′-biphenyl.

### 3rd step: Synthesis of Compound A3

9.5 g (48%) of Compound A3 was synthesized and purified in the same method as in the 3^{rd} step of Preparation Synthesis Example 1 except that Int 3-1-b was used instead of Int 1-1-b.

LC-Mass (theoretical value: 546.20 g/mol, measured value: M+=547.88 g/mol)

### Preparation Synthesis Example 4: Synthesis of Compound A4

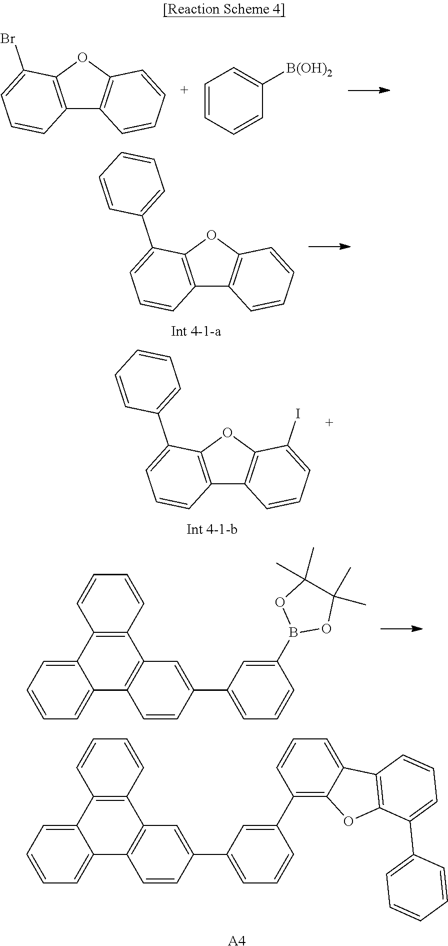

### 1st step: Synthesis of Intermediate Int 4-1-a

41.8 g (169.2 mmol) of 4-bromodibenzofuran, 24.8 g (203.3 mmol) of phenylboronic acid, 46.8 g (338.3 mmol) of K₂CO₃, and 5.9 g (5.1 mmol) of Pd(PPh₃)₄ were suspended in 340 ml of THF and 170 ml of distilled water under a nitrogen flow and then, stirred under reflux for 8 hours. When a reaction was completed, the resultant was extracted with DCM and treated through column chromatography (hexane:DCM (20%)), obtaining 28.5 g (69%) of Intermediate Int 4-1-a as a solid.

### 2nd step: Synthesis of Intermediate Int 4-1-b

28.5 g (116.7 mmol) of Intermediate Int 4-1-a was dissolved in 250 ml of THF in a round-bottomed flask under a nitrogen flow and then, stirred at −78° C. for 30 minutes. 51.3 ml (128.3 mmol) of n-butyllithium (2.5 M solution) was slowly added thereto in a dropwise fashion for 1 hour and then, additionally stirred for 4 hours. 29.6 g (116.7 mmol) of an iodine solution diluted in THF was slowly added thereto in a dropwise fashion at −78° C. and then, stirred at ambient temperature for 4 hours. When a reaction was completed, after adding a saturated sodium bicarbonate aqueous solution and DCM thereto and then, stirring the mixture for 1 hour, an organic layer was separated therefrom, filtered through silica gel pad, and treated under a reduced pressure to remove a solvent, obtaining 34.6 g (80%) of Intermediate Int 4-1-b.

### 3rd step: Synthesis of Compound A4

29.2 g (81%) of Compound A4 was synthesized and purified in the same manner as in the 3^{rd} step of Preparation Synthesis Example 1 except that Intermediate Int 4-1-b was used.

LC-Mass (theoretical value: 546.66 g/mol, measured value: M+=547.64 g/mol)

### Preparation Synthesis Example 5: Synthesis of Compound A5

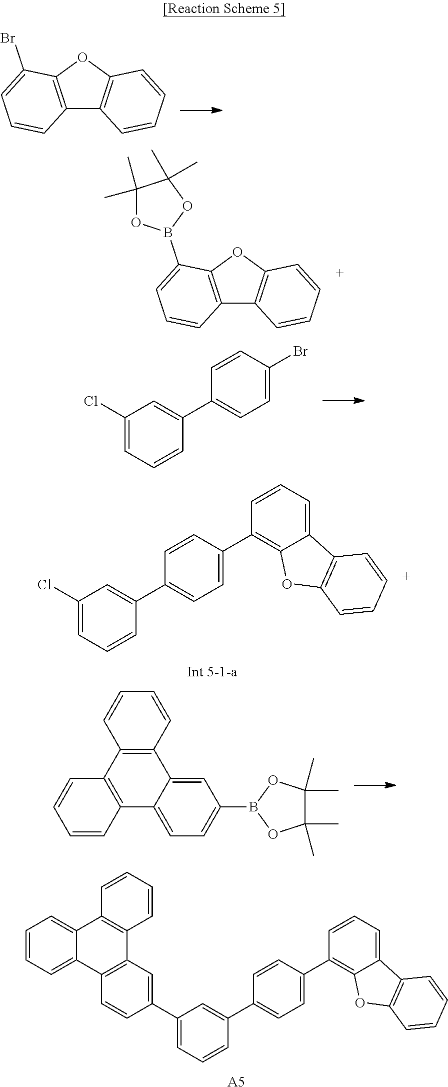

### 1st step: Synthesis of Intermediate Int 5-1-a

12.2 g (68%) of Intermediate Int 5-1-a was synthesized and purified in the same manner as in the 1st step and 2nd step of Preparation Synthesis Example 1 except that 4-bromodibenzofuran was used instead of the 1-bromodibenzofuran.

### 2nd step: Synthesis of Compound A5

9.1 g (56%) of Compound A5 was synthesized and purified in the same manner as in the 3rd step of Preparation Synthesis Example 1 except that Intermediate Int 5-1-a was used.

LC-Mass (theoretical value: 546.20 g/mol, measured value: M+=547.82 g/mol)

### Preparation Synthesis Example 6: Synthesis of Compound A6

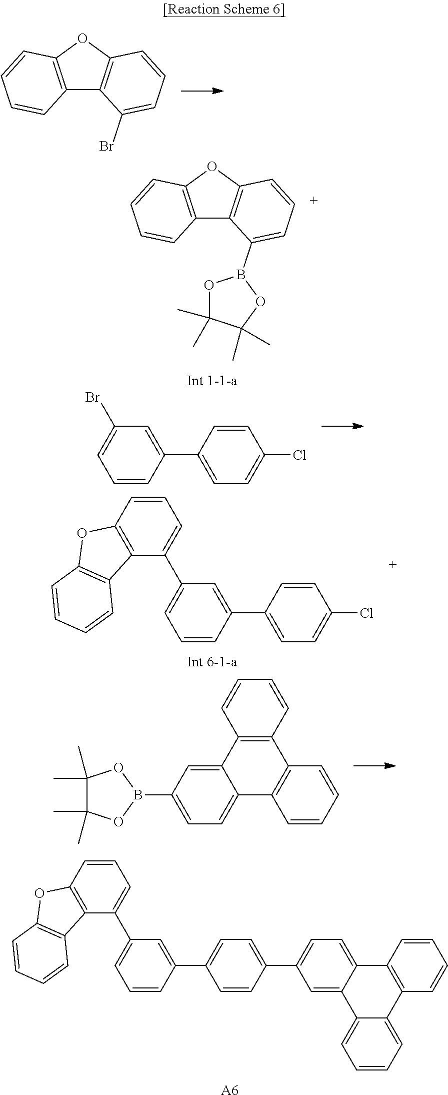

### 1st step: Synthesis of Intermediate Int 6-1-a

11.9 g (67%) of Intermediate Int 6-1-a was synthesized and purified in the same manner as in the 1st step and 2nd step of Preparation Synthesis Example 1 except that 3-bromo-4′-chloro-1,1′-biphenyl was used instead of the 4-bromo-3′-chloro-1,1′-biphenyl.

### 2nd step: Synthesis of Compound A6

8.9 g (55%) of Compound A6 was synthesized and purified in the same manner as in the 3rd step of Preparation Synthesis Example 1 except that Intermediate Int 6-1-a was used.

LC-Mass (theoretical value: 546.20 g/mol, measured value: M+=547.82 g/mol)

### Synthesis Example 1: Synthesis of Compound 33

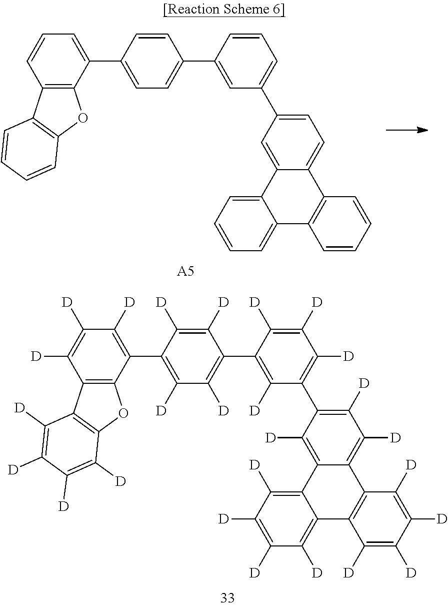

20 g of Compound A5 was put in a round-bottomed flask, and 440 ml of benzene-D6 was added thereto and then, stirred. 16 ml of triflic acid was added thereto and then, refluxed. After 24 hours, the resultant was cooled to ambient temperature, and D20 was added thereto and then, stirred for 30 minutes. A product therefrom was dissolved in an excess of DCM and then, neutralized with a K₃PO₄ aqueous solution. After removing an aqueous layer therefrom, an organic layer was silica gel-filtered, treated to remove a solvent, and recrystallized, obtaining 15 g of Compound 33 (white solid, LC-Mass Mz 572.4, C₄₂D₂₆O₂).

### Synthesis Example 2: Synthesis of Compound 36

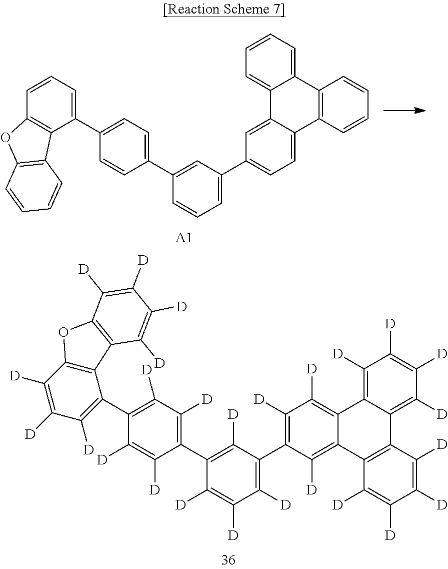

16 g of Compound 36 (white solid, LC-Mass Mz 572.4, C₄₂D₂₆O₂) was synthesized in the same manner as in Synthesis Example 1 except that 20 g of Compound A1 was used instead of Compound A5.

### Synthesis Example 3: Synthesis of Compound 54

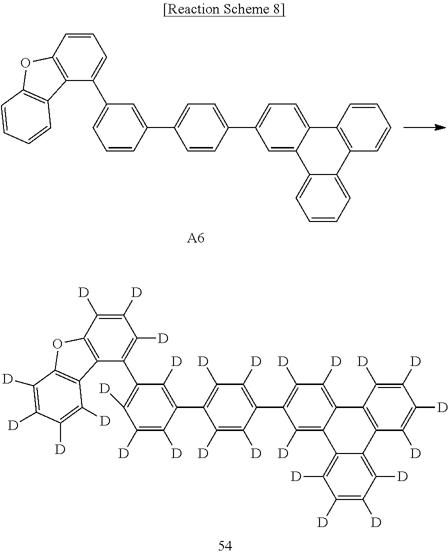 [SMILES: c1cc(-c2ccc(-c3ccc4c5ccccc5c5ccccc5c4c3)cc2)cc(-c2cccc3oc4ccccc4c23)c1 ; "A6" ; [2H]c1c([2H])c(-c2c([2H])c([2H])c(-c3c([2H])c([2H])c4c5c([2H])c([2H])c([2H])c([2H])c5c5c([2H])c([2H])c([2H])c([2H])c5c4c3[2H])c([2H])c2[2H])c([2H])c(-c2c([2H])c([2H])c([2H])c3oc4c([2H])c([2H])c([2H])c([2H])c4c23)c1[2H]]

17 g of Compound 54 (white solid, LC-Mass Mz 572.4, C₄₂D₂₆O₂) was synthesized in the same manner as in Synthesis Example 1 except that 20 g of Compound A6 was used instead of Compound A5.

### Synthesis Example 4: Synthesis of Compound 48

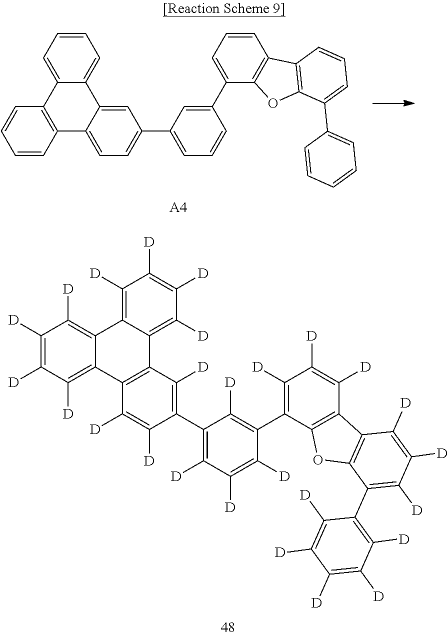

14 g of Compound 48 (white solid, LC-Mass Mz 572.4, C₄₂D₂₆O₂) was synthesized in the same manner as in Synthesis Example 1 except that 20 g of Compound A4 was used instead of Compound A5.

(Manufacture of Organic Light Emitting Diode)

### EXAMPLE 1

A glass substrate coated with ITO (indium tin oxide) was ultrasonically washed with distilled water. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor. This prepared ITO transparent electrode was used as an anode, Compound A doped with 3% NDP-9 (Novaled GmbH) was vacuum-deposited on the ITO substrate to form a 100 Å-thick hole injection layer, and Compound A is deposited on the hole injection layer to a thickness of 1,350 Å to form a hole transport layer. Compound B was deposited on the hole transport layer to a thickness of 350 Å to form a hole transport auxiliary layer. On the hole transport auxiliary layer, Compound 33 obtained in Synthesis Example 1 was used as a host and 7 wt % of PhGD was used as a dopant to form a 400 Å-thick light emitting layer by vacuum deposition. Subsequently, Compound C was deposited to form a 50 Å-thick electron transport auxiliary layer on the light emitting layer, and Compound D and LiQ were simultaneously vacuum-deposited in a weight ratio of 1:1 to form a 300 Å-thick electron transport layer. On the electron transport layer, LiQ and Al were sequentially vacuum-deposited to be 15 Å-thick and 1,200 Å-thick, manufacturing an organic light emitting diode.

The structure was ITO/Compound A (3% NDP-9 doping, 100 Å)/Compound A (1,350 Å)/Compound B (350 Å)/EML [93 wt % of host (Compound 33): 7 wt % of PhGD] (400 Å)/Compound C (50 Å)/Compound D:LiQ (300 Å)/LiQ (15 Å) Al (1,200 Å).

### Compound A: N-(biphenyl-4-yl)-9,9-dimethyl-N-(4-(9-phenyl-9H-carbazol-3-yl)phenyl)-9H-fluoren-2-amine

### Compound B: N-[4-(4-dibenzofuranyl)phenyl]-N-[4-(9-phenyl-9H-fluoren-9-yl)phenyl][1,1′-biphenyl]-4-amine

### Compound C: 2,4-diphenyl-6-(4′,5′,6′-triphenyl[1,1′:2′,1″:3″,1′″:3′″,1″-quinquephenyl]-3″″-yl)-1,3,5-triazine

### Compound D: 2-(1,1′-Biphenyl-4-yl)-4-(9,9-diphenylfluoren-4-yl)-6-phenyl-1,3,5-triazine

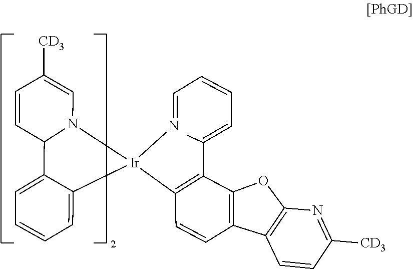

### COMPARATIVE EXAMPLE 1

A diode of Comparative Example 1 was manufactured in the same manner as in Example 1 except that the host was changed as described in Table 1.

Evaluation: Confirmation of Life-span Enhancement Effect

While luminance (cd/m²) was maintained at 6,000 cd/m², time taken until luminous efficiency (cd/A) was reduced to 97% was measured.

A relative value of Example 1 with reference to life-span of Comparative

Example 1 is shown in Table 1.

TABLE 1

| | Host | Life-spanT97 (%) |
|---|---|---|
| Example 1 | Compound 33 | 126 |
| Comparative | Compound A5 | 100 |
| Example 1 | | |

Referring to Table 1, an organic light emitting diode according to Example 1 exhibited significantly improved life-span characteristics, compared with the organic light emitting diode according to Comparative Example 1.

One or more embodiments may provide a compound for an organic optoelectronic device capable of implementing an organic optoelectronic device having high efficiency and a long life-span.

An organic optoelectronic device having high efficiency long life-span may be realized.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic optoelectronic device, the compound being represented by Chemical Formula 1: 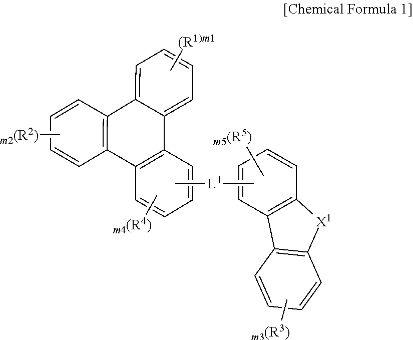
wherein, in Chemical Formula 1,
X¹ is O or S,
L^{l} is a single bond, a substituted or unsubstituted C6 to C20 arylene group, a substituted or unsubstituted C2 to C20 heterocyclic group, or a combination thereof,
R¹ to R⁵ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, a halogen, a cyano group, or a combination thereof,
m1 to m3 are each independently an integer of 1 to 4,
m4 and m5 are each independently an integer 1 to 3, and
provided that at least one of the following conditions is met:
L¹ is a C6 to C20 arylene group substituted with at least one deuterium or a C2 to C20 heterocyclic group substituted with at least one deuterium; or
at least one of R¹ to R⁵ is deuterium, a C6 to C30 aryl group substituted with at least one deuterium, or a C2 to C30 heterocyclic group substituted with at least one deuterium.

2. The compound as claimed in claim 1, wherein:
L¹ is a C6 to C20 arylene group substituted with at least one deuterium or a C2 to C20 heterocyclic group substituted with at least one deuterium, and
at least one of R¹ to R⁵ is deuterium, a C6 to C30 aryl group substituted with at least one deuterium, or a C2 to C30 heterocyclic group substituted with at least one deuterium.

3. The compound as claimed in claim 1, wherein L¹ is a phenylene group substituted with at least one deuterium, a biphenylene group substituted with at least one deuterium, a terphenylene group substituted with at least one deuterium, a naphthylene group substituted with at least one deuterium, an anthracenylene group substituted with at least one deuterium, a phenanthrenylene group substituted with at least one deuterium, a triphenylenylene group substituted with at least one deuterium, a fluorenylene group substituted with at least one deuterium, a dibenzofuranylene group substituted with at least one deuterium, or a dibenzothiophenylene group substituted with at least one deuterium.

4. The compound as claimed in claim 1, wherein at least one of R¹ to R⁵ is deuterium, a phenyl group substituted with at least one deuterium, a biphenyl group substituted with at least one deuterium, a terphenyl group substituted with at least one deuterium, a naphtyl group substituted with at least one deuterium, an anthracenyl group substituted with at least one deuterium, a phenanthrenyl group substituted with at least one deuterium, a triphenylene group substituted with at least one deuterium, a fluorenyl group substituted with at least one deuterium, a carbazolyl group substituted with at least one deuterium, a dibenzofuranyl group substituted with at least one deuterium, or a dibenzothiophenyl group substituted with at least one deuterium.

5. The compound as claimed in claim 1, wherein:
Chemical Formula 1 is represented by one of Chemical Formula 1-1 to Chemical Formula 1-4: 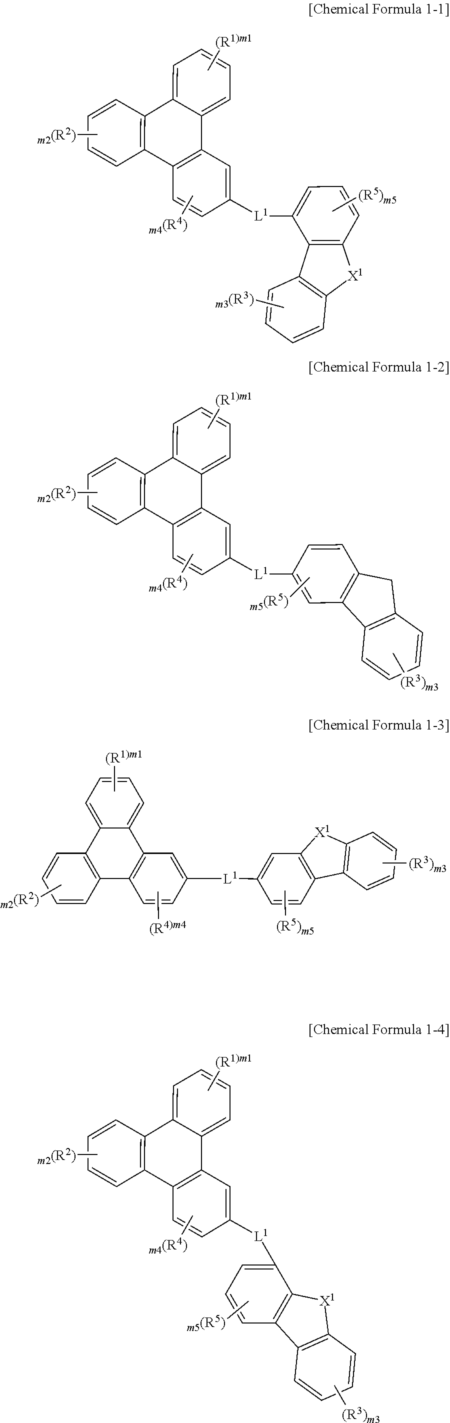
in Chemical Formula 1-1 to Chemical Formula 1-4, X¹, L¹, R¹ to R⁵, and m1 to m5 are defined the same as those of Chemical Formula 1.

6. The compound as claimed in claim 1, wherein the compound represented by Chemical Formula 1 is a compound of Group 1:

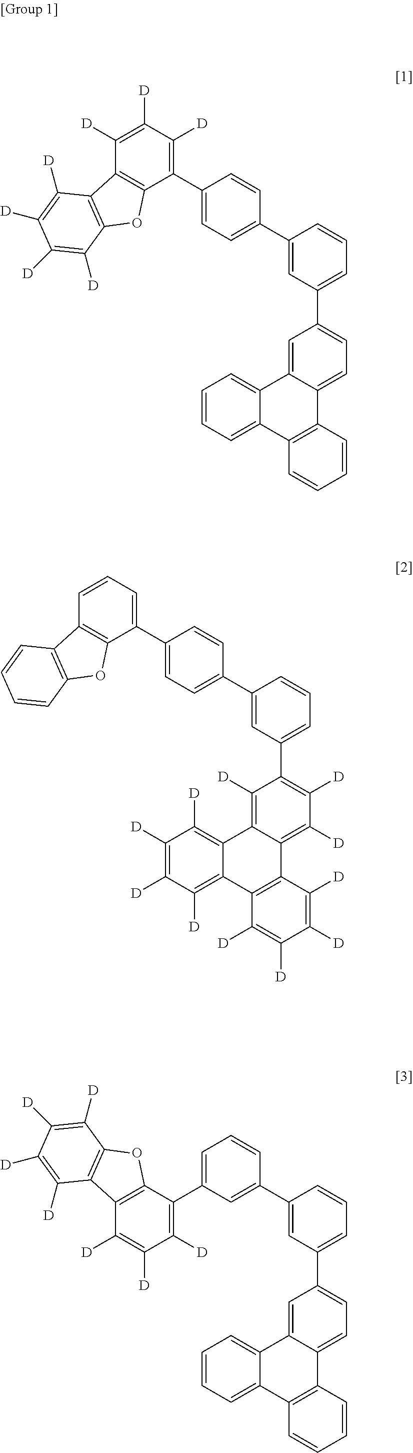 [SMILES: [2H]c1c([2H])c([2H])c2c(oc3c(-c4ccc(-c5cccc(-c6ccc7c8ccccc8c8ccccc8c7c6)c5)cc4)c([2H])c([2H])c([2H])c32)c1[2H] ; [2H]c1c([2H])c([2H])c2c(c1[2H])c1c([2H])c([2H])c([2H])c([2H])c1c1c([2H])c(-c3cccc(-c4ccc(-c5cccc6c5oc5ccccc56)cc4)c3)c([2H])c([2H])c21 ; [2H]c1c([2H])c([2H])c2c(oc3c(-c4cccc(-c5cccc(-c6ccc7c8ccccc8c8ccccc8c7c6)c5)c4)c([2H])c([2H])c([2H])c32)c1[2H]]

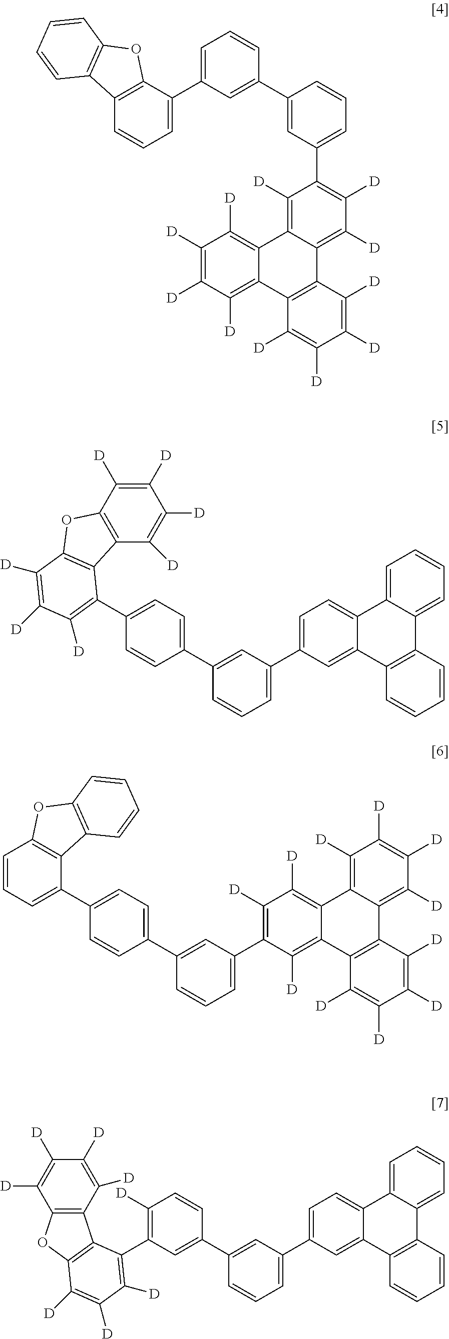

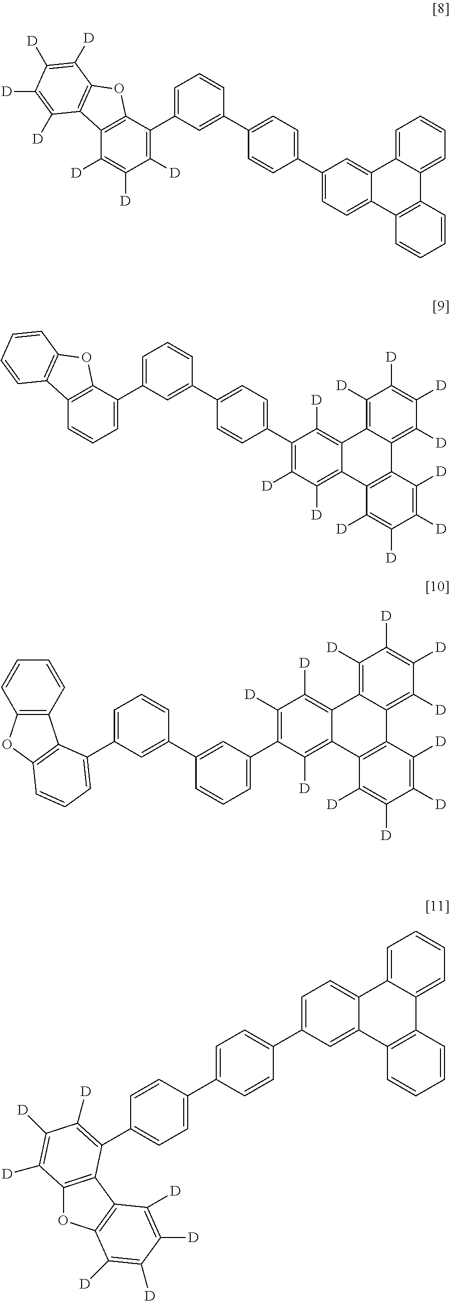 [SMILES: [2H]c1c([2H])c([2H])c2c(oc3c(-c4cccc(-c5ccc(-c6ccc7c8ccccc8c8ccccc8c7c6)cc5)c4)c([2H])c([2H])c([2H])c32)c1[2H] ; [2H]c1c([2H])c([2H])c2c(c1[2H])c1c([2H])c([2H])c([2H])c([2H])c1c1c([2H])c(-c3ccc(-c4cccc(-c5cccc6c5oc5ccccc56)c4)cc3)c([2H])c([2H])c21 ; [2H]c1c([2H])c([2H])c2c(c1[2H])c1c([2H])c([2H])c([2H])c([2H])c1c1c([2H])c(-c3cccc(-c4cccc(-c5cccc6oc7ccccc7c56)c4)c3)c([2H])c([2H])c21 ; [2H]c1c([2H])c([2H])c2c(oc3c([2H])c([2H])c([2H])c(-c4ccc(-c5ccc(-c6ccc7c8ccccc8c8ccccc8c7c6)cc5)cc4)c32)c1[2H]]

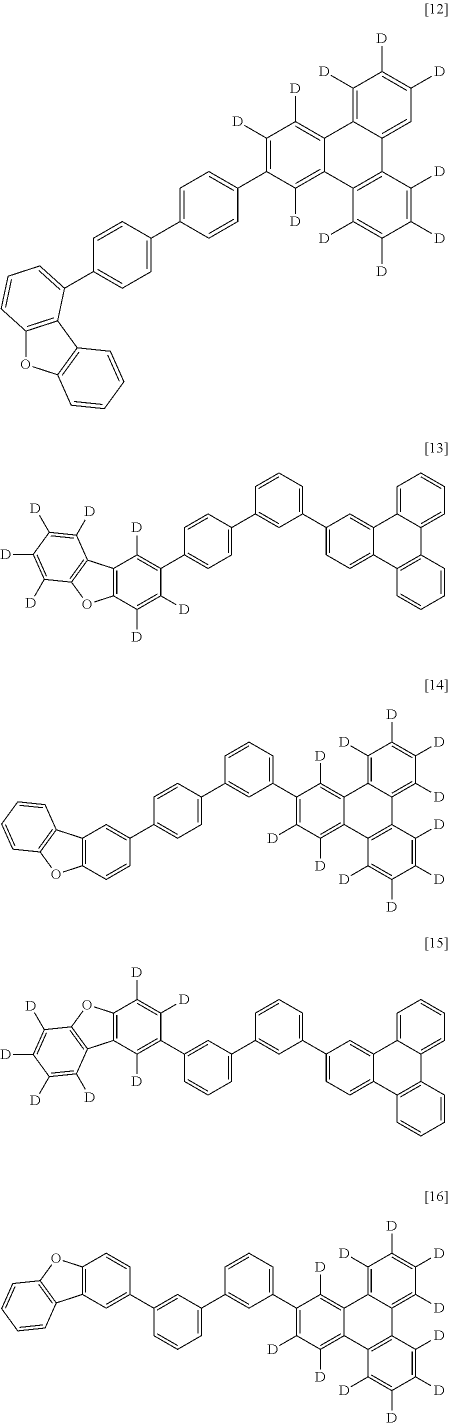

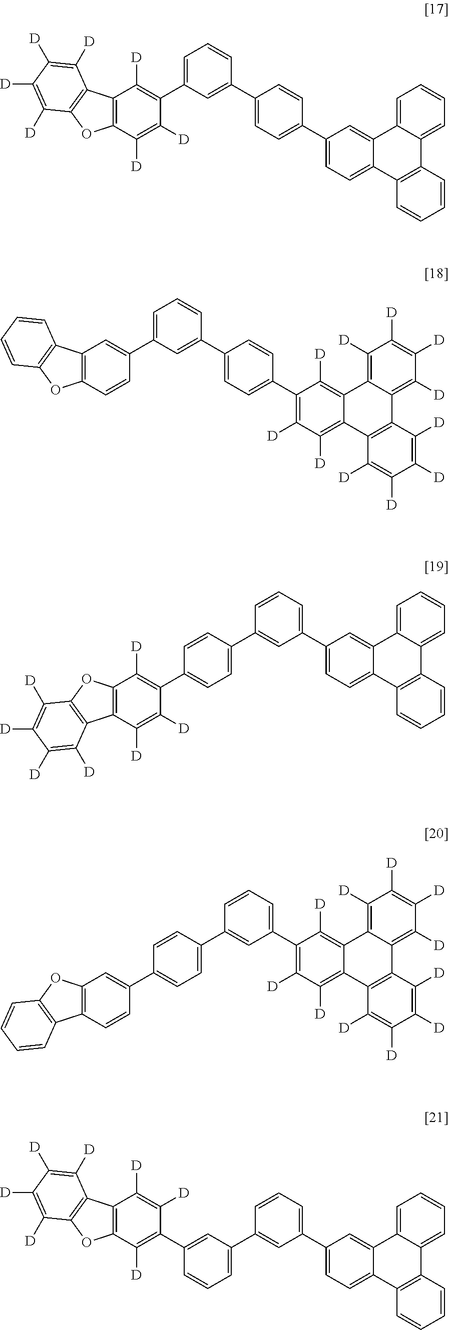

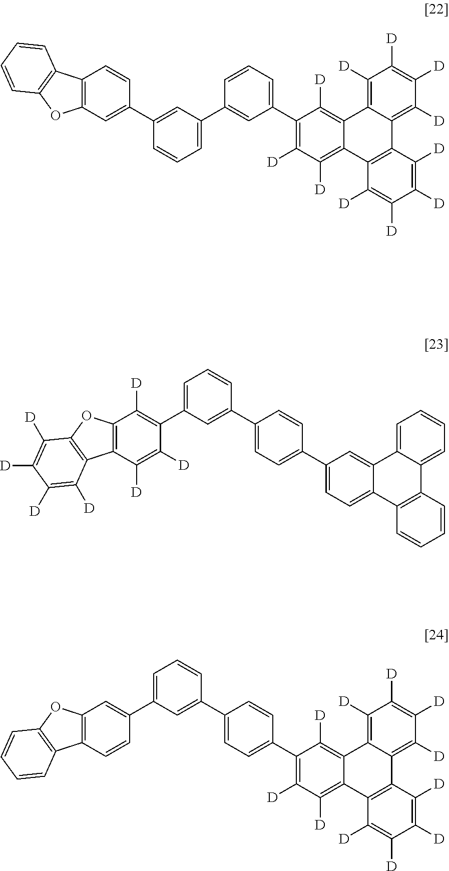 [SMILES: [2H]c1c([2H])c([2H])c2c(c1[2H])c1c([2H])c([2H])c([2H])c([2H])c1c1c([2H])c(-c3cccc(-c4cccc(-c5ccc6c(c5)oc5ccccc56)c4)c3)c([2H])c([2H])c21 ; [2H]c1c([2H])c([2H])c2c(oc3c([2H])c(-c4cccc(-c5ccc(-c6ccc7c8ccccc8c8ccccc8c7c6)cc5)c4)c([2H])c([2H])c32)c1[2H] ; [2H]c1c([2H])c([2H])c2c(c1[2H])c1c([2H])c([2H])c([2H])c([2H])c1c1c([2H])c(-c3ccc(-c4cccc(-c5ccc6c(c5)oc5ccccc56)c4)cc3)c([2H])c([2H])c21]

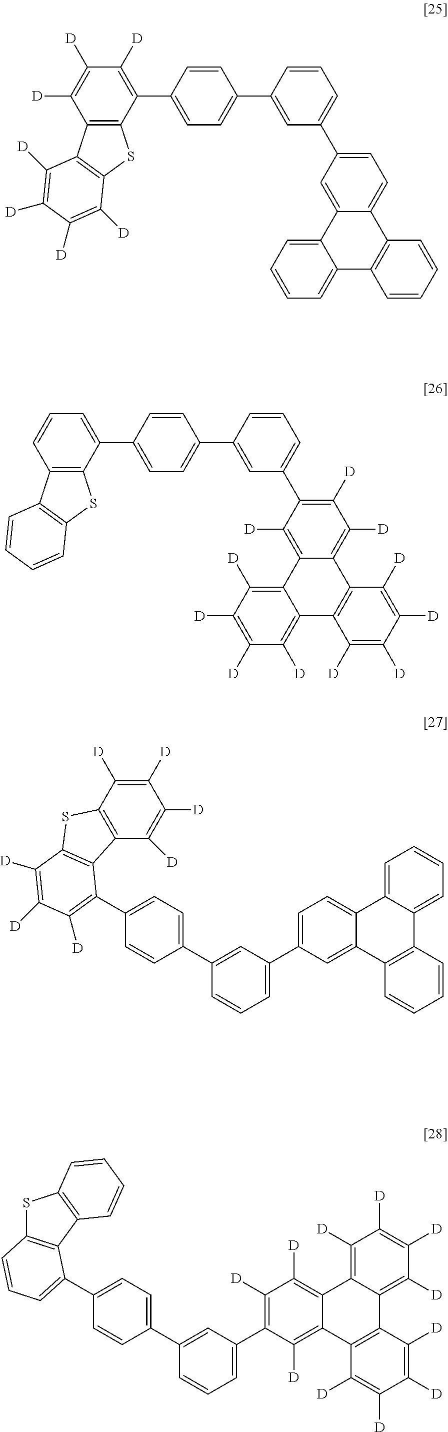 [SMILES: [2H]c1c([2H])c([2H])c2c(sc3c(-c4ccc(-c5cccc(-c6ccc7c8ccccc8c8ccccc8c7c6)c5)cc4)c([2H])c([2H])c([2H])c32)c1[2H] ; [2H]c1c([2H])c([2H])c2c(c1[2H])c1c([2H])c([2H])c([2H])c([2H])c1c1c([2H])c(-c3cccc(-c4ccc(-c5cccc6c5sc5ccccc56)cc4)c3)c([2H])c([2H])c21 ; [2H]c1c([2H])c([2H])c2c(sc3c([2H])c([2H])c([2H])c(-c4ccc(-c5cccc(-c6ccc7c8ccccc8c8ccccc8c7c6)c5)cc4)c32)c1[2H] ; [2H]c1c([2H])c([2H])c2c(c1[2H])c1c([2H])c([2H])c([2H])c([2H])c1c1c([2H])c(-c3cccc(-c4ccc(-c5cccc6sc7ccccc7c56)cc4)c3)c([2H])c([2H])c21]

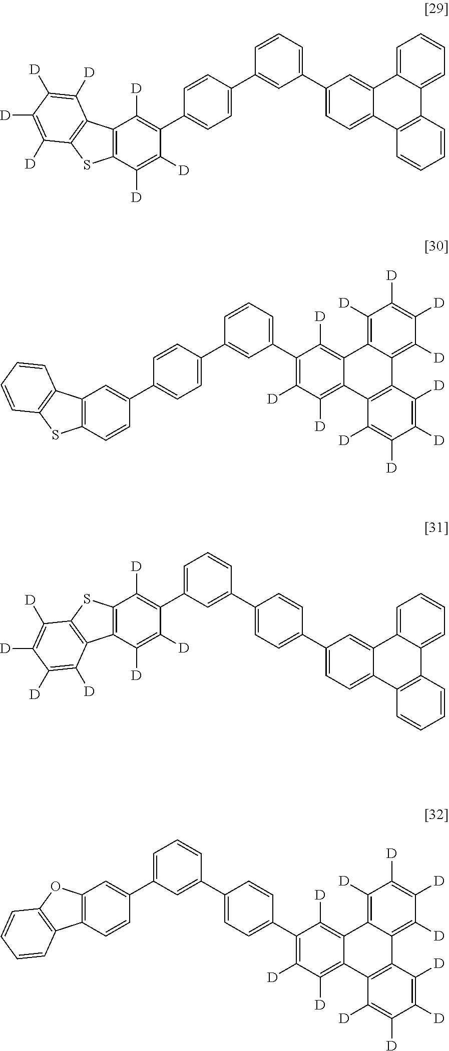 [SMILES: [2H]c1c([2H])c([2H])c2c(sc3c([2H])c([2H])c(-c4ccc(-c5cccc(-c6ccc7c8ccccc8c8ccccc8c7c6)c5)cc4)c([2H])c32)c1[2H] ; [2H]c1c([2H])c([2H])c2c(c1[2H])c1c([2H])c([2H])c([2H])c([2H])c1c1c([2H])c(-c3cccc(-c4ccc(-c5ccc6sc7ccccc7c6c5)cc4)c3)c([2H])c([2H])c21 ; [2H]c1c([2H])c([2H])c2c(sc3c([2H])c(-c4cccc(-c5ccc(-c6ccc7c8ccccc8c8ccccc8c7c6)cc5)c4)c([2H])c([2H])c32)c1[2H] ; [2H]c1c([2H])c([2H])c2c(c1[2H])c1c([2H])c([2H])c([2H])c([2H])c1c1c([2H])c(-c3ccc(-c4cccc(-c5ccc6c(c5)oc5ccccc56)c4)cc3)c([2H])c([2H])c21]

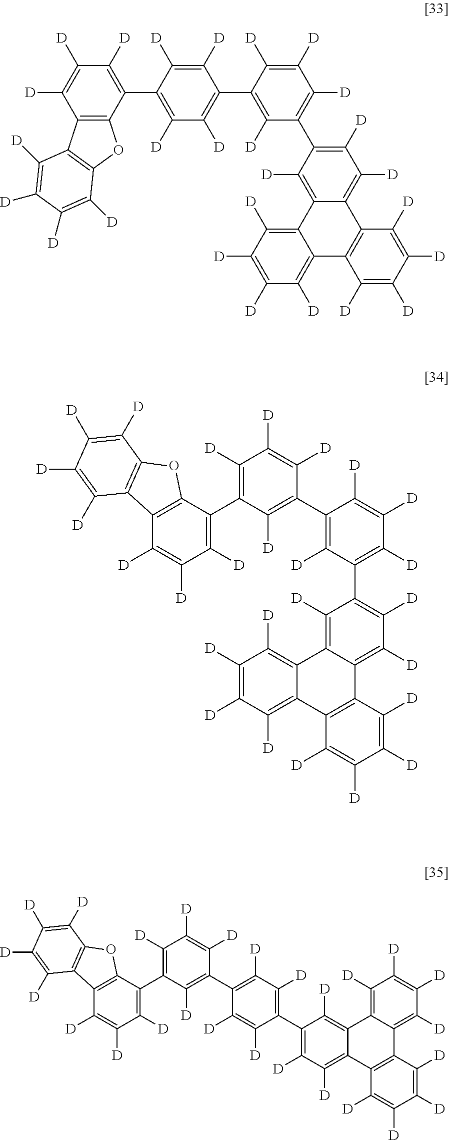

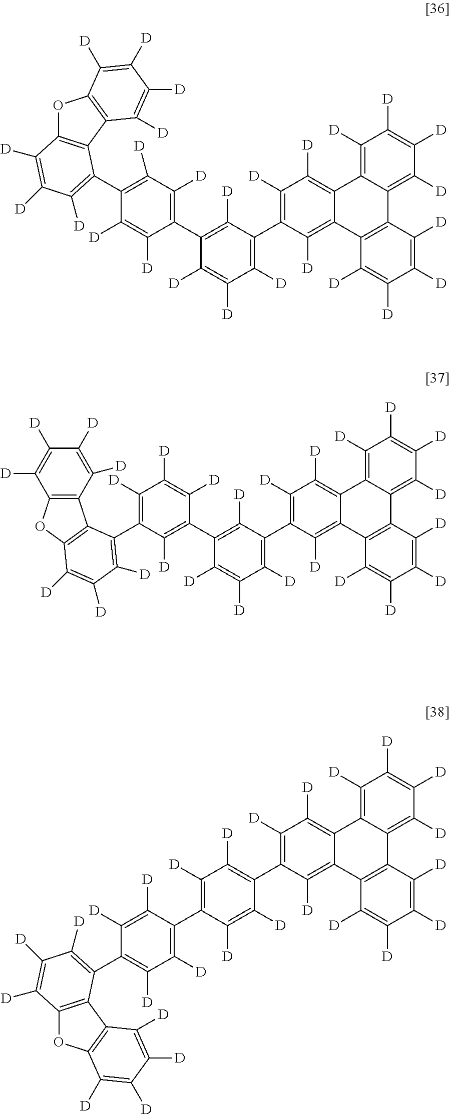

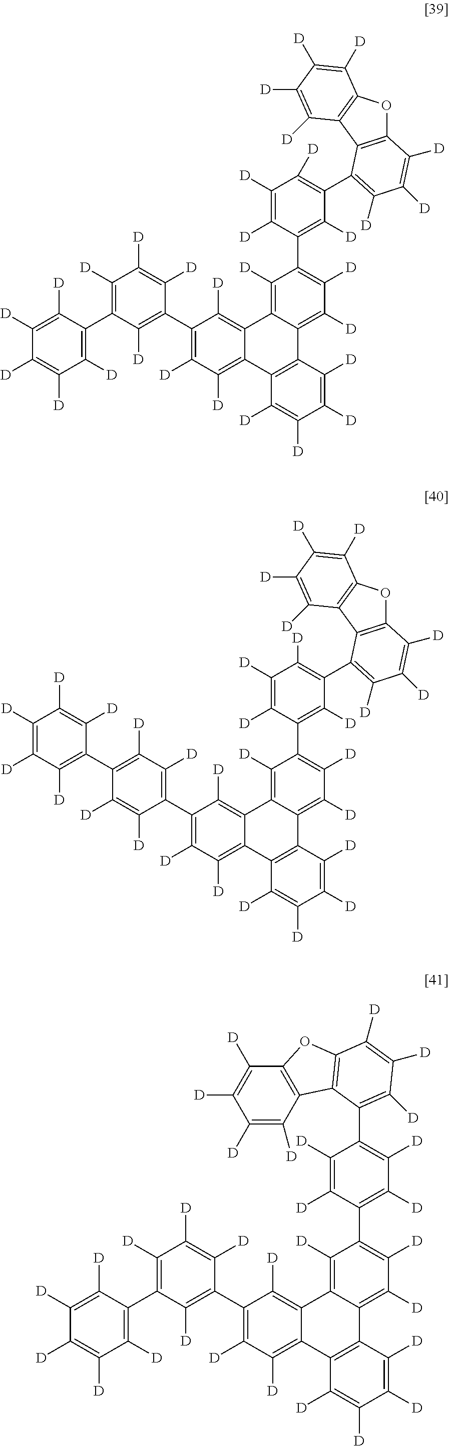

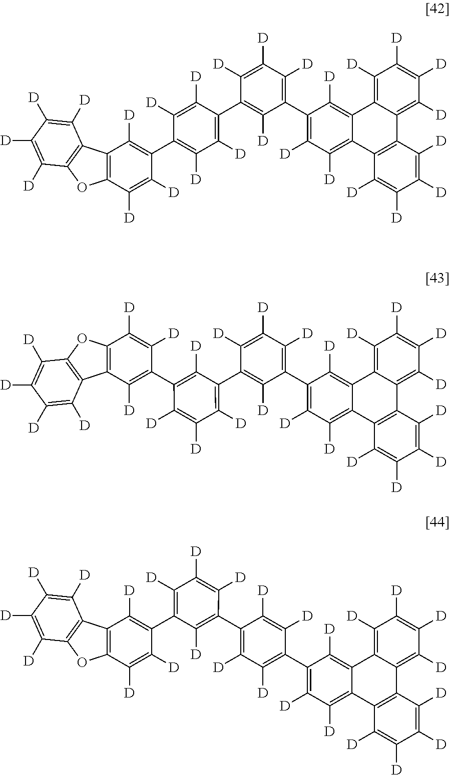

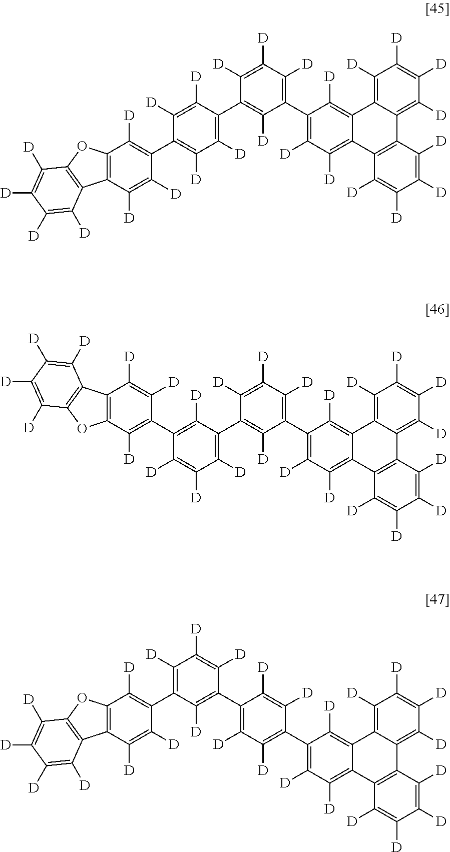

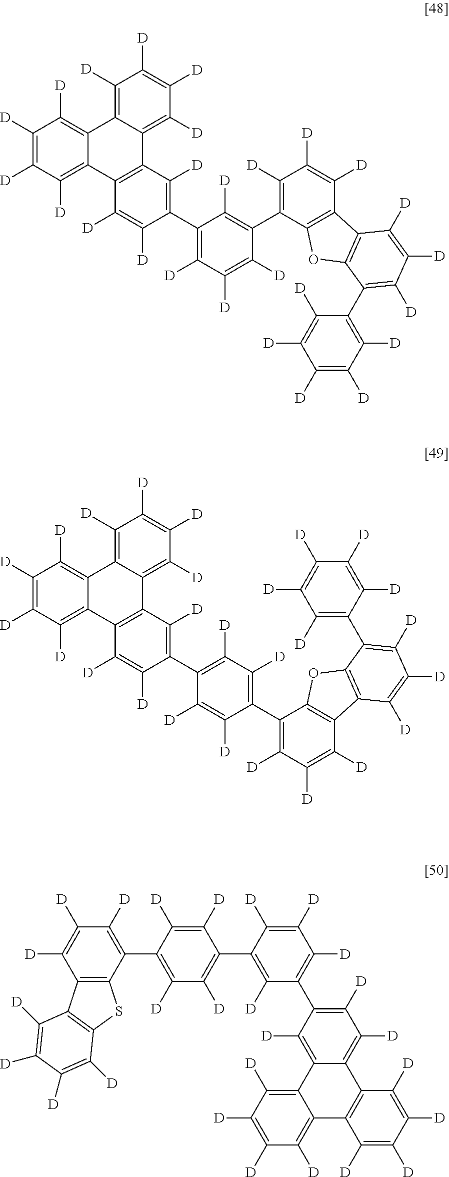

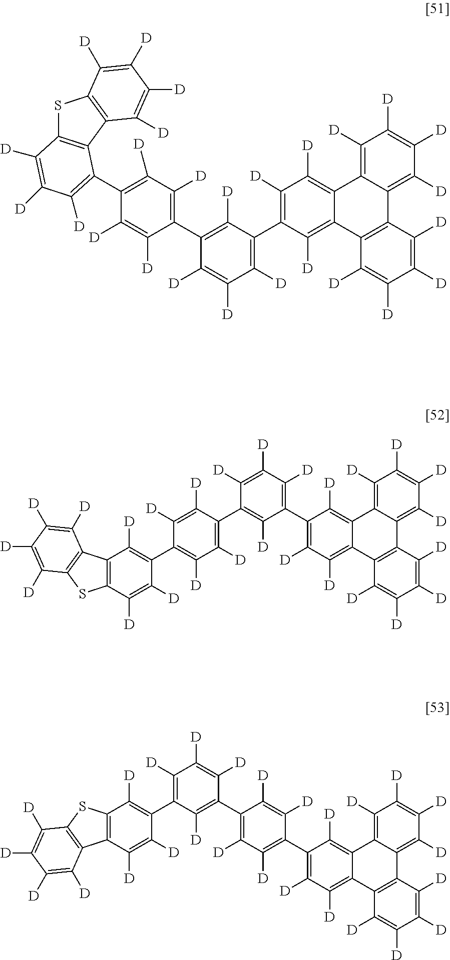

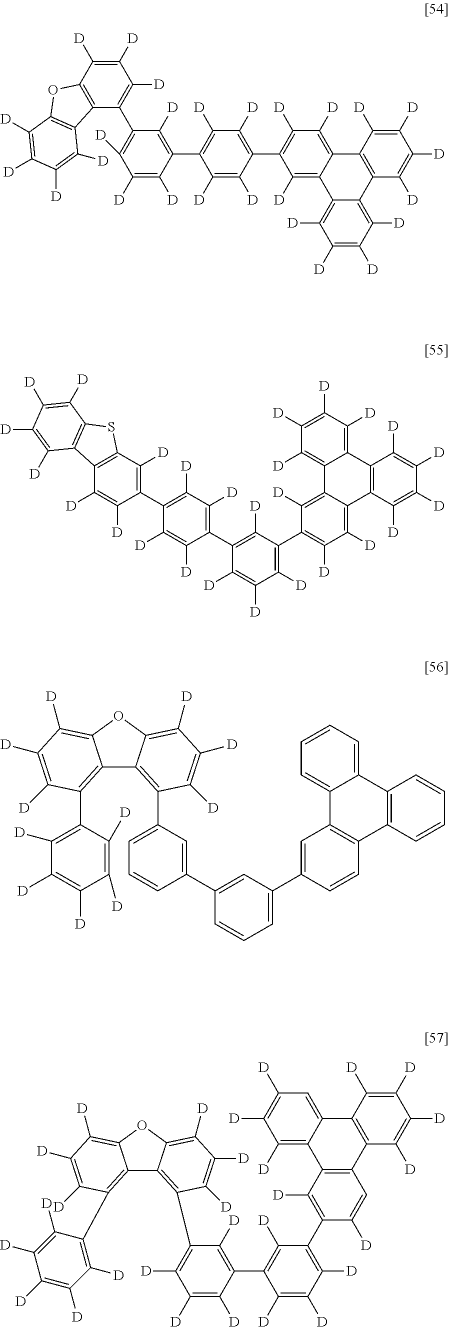

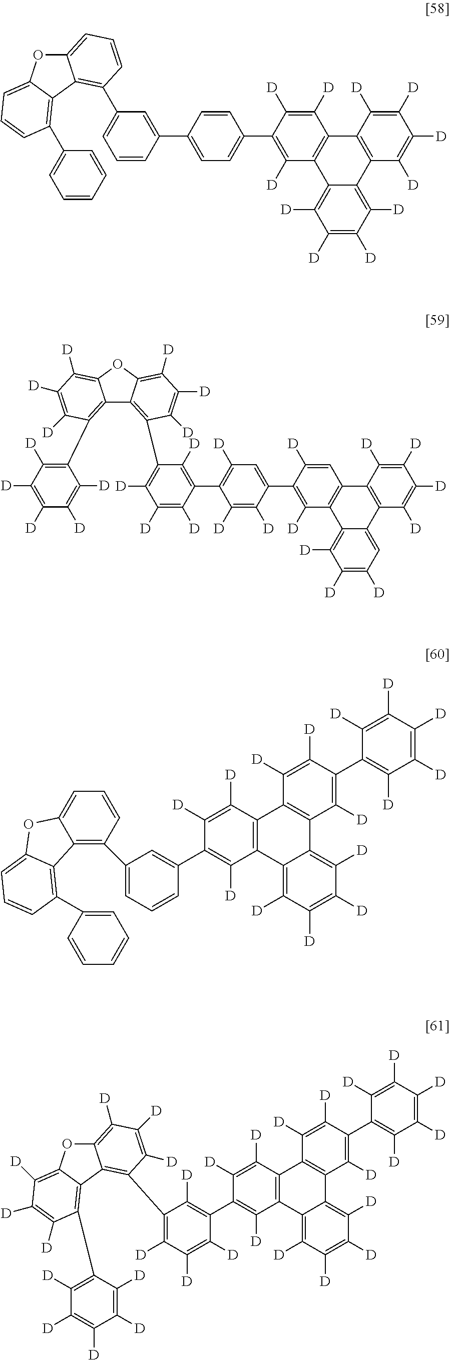

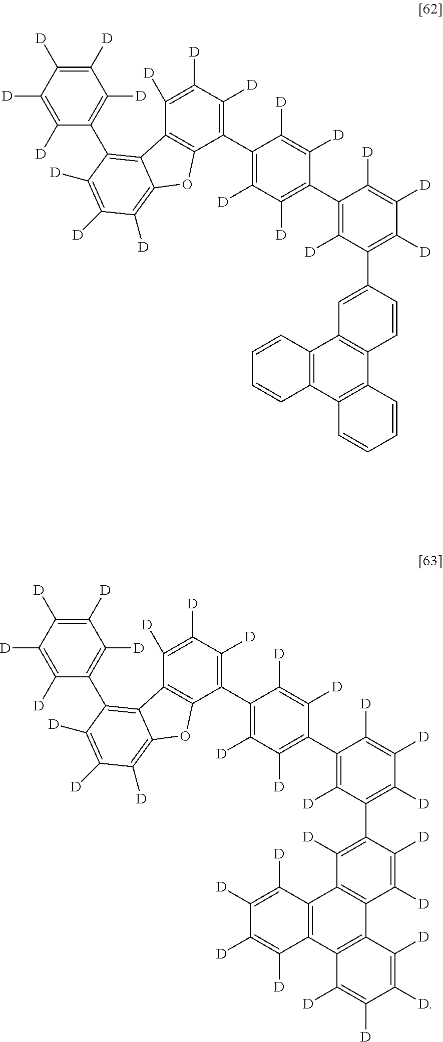

7. An organic optoelectronic device, comprising:
an anode and a cathode facing each other, and
at least one organic layer between the anode and the cathode,
wherein the at least one organic layer includes the compound for an organic optoelectronic device as claimed in claim 1.

8. The organic optoelectronic device as claimed in claim 7, wherein:
the at least one organic layer includes a light emitting layer, and
the light emitting layer includes the compound for an organic optoelectronic device.

9. A display device comprising the organic optoelectronic device as claimed in claim 7.
